SEQUENCE LISTING (14 sequences)

SEQ ID NO: 1 - DNA, 3675 bp, Homo sapiens
tctggctcgggctctggctcgggaggcgatcgtggccgctgccgccgccagagccggccggagtcggcccggagccgcgtccacgcggagcgggcgggtgggagctggcggctgggcccctgcggcgcccccgcggcctgccccgcgtccgcctccgggccgccgagccgcagccgccgagatgggggccgccccgggccgcgcccccgccgggtcccgcccgccgcgctgccgctgagcgcatgggcccggaccgcgccgcgccgctccgggagccgggcccggggtcccgccaccaccgcgcgcgggacagattgattcactttggagctgtaagtactgatgtattagggtgcagcgctcattgttccttgacgcagagtcccaaaatgaatatccaagagcagggtttccccttggacctcggagcaagtttcaccgaagatgctccccgacccccagtgcctggtgaggagggagaactggtgtccacagacccgaggcccgccagctacagtttctgctccgggaaaggtgttggcattaaaggtgagacttcgacggccactccgaggcgctcggatctggacctggggtatgagcctgagggcagtgcctcccccaccccaccatacttgaagtgggctgagtcactgcattccctgctggatgaccaagatgggataagcctgttcaggactttcctgaagcaggagggctgtgccgacttgctggacttctggtttgcctgcactggcttcaggaagctggagccctgtgactcgaacgaggagaagaggctgaagctggcgagagccatctaccgaaagtacattcttgataacaatggcatcgtgtcccggcagaccaagccagccaccaagagcttcataaagggctgcatcatgaagcagctgatcgatcctgccatgtttgaccaggcccagaccgaaatccaggccactatggaggaaaacacctatccctccttccttaagtctgatatttatttggaatatacgaggacaggctcggagagccccaaagtctgtagtgaccagagctctgggtcagggacagggaagggcatatctggatacctgccgaccttaaatgaagatgaggaatggaagtgtgaccaggacatggatgaggacgatggcagagacgctgctccccccggaagactccctcagaagctgctcctggagacagctgccccgagggtctcctccagtagacggtacagcgaaggcagagagttcaggtatggatcctggcgggagccagtcaacccctattatgtcaatgccggctatgccctggccccagccaccagtgccaacgacagcgagcagcagagcctgtccagcgatgcagacaccctgtccctcacggacagcagcgtggatgggatccccccatacaggatccgtaagcagcaccgcagggagatgcaggagagcgtgcaggtcaatgggcgggtgcccctacctcacattccccgcacgtaccgggtgccgaaggaggtccgcgtggagcctcagaagttcgcggaggagctcatccaccgcctggaggctgtgcagcgcacgcgggaggccgaggagaagctggaggagcggctgaagcgcgtgcgcatggaggaggaaggtgaggacggcgatccatcgtcagggcccccagggccgtgtcacaagctgcctcccgcccccgcttggcaccacttcccgccccgctgtgtggacatgggctgtgccgggctccgggatgcacacgaggagaaccctgagagcatcctggacgagcacgtacagcgtgtgctgaggacacctggccgccagtcgcctgggcctggccatcgctccccggacagtgggcacgtggccaagatgccagtggcactggggggtgccgcctcggggcacgggaagcacgtacccaagtcaggggcgaagctggacgcggccggcctgcaccaccaccgacacgtccaccaccacgtccaccacagcacagcccggcccaaggagcaggtggaggccgaggccacccgcagggcccagagcagcttcgcctggggcctggaaccacacagccatggggcaaggtcccgaggctactcagagagtgttggcgctgcccccaacgccagtgatggcctcgcccacagtgggaaggtgggcgtggcgtgcaaaagaaatgccaagaaggctgagtcggggaagagcgccagcaccgaggtgccaggtgcctcggaggatgcggagaagaaccagaaaatcatgcagtggatcattgagggggaaaaggagatcagcaggcaccgcaggaccggccacgggtcttcggggacgaggaagccacagccccatgagaactccagacccttgtcccttgagcacccctgggccggccctcagctccggacctccgtgcagccctcccacctcttcatccaagaccccaccatgccaccccacccagctcccaaccccctaacccagctggaggaggcgcgccgacgtctggaggaggaagaaaagagagccagccgagcaccctccaagcagaggtatgtgcaggaggttatgcggcggggacgcgcctgcgtcaggccagcgtgcgcgccggtgctgcacgtggtaccagccgtgtcggacatggagctctccgagacagagacaagatcgcagaggaaggtgggcggcgggagtgcccagccgtgtgacagcatcgttgtggcgtactacttctgcggggaacccatcccctaccgcaccctggtgaggggccgcgctgtcaccctgggccagttcaaggagctgctgaccaaaaagggcagctacagatactacttcaagaaagtgagcgacgagtttgactgtggggtggtgtttgaggaggttcgagaggacgaggccgtcctgcccgtctttgaggagaagatcatcggcaaagtggagaaggtggactgataggctggtgggctggccgctgtgccaggcgaggcccttggcgggcacgggtgtcacggccaggcagatgacctcgtactcaggagcccgatggggaacagtgttgggtgtaccacccatccctgtggtctacccgtgtctagaggcaggtagggggtccctccaagtggtccacaagcttctgtcctgcccccaaggaggcagcctggaccactcctcatagcaatacttggagggcccagcccaagtgaggcagccgaggtccctgctgccagcttcaggtgacccccccccatcccccggcacctcccttgggcacgtgtgctgggatctactttccctctgggatttgcccacgtacccaggtctgggtggggcccaggcccggatgcagaggcctgcagggcctctgtcaattgtacgcgccaccgagtgccttcaacacagcttgtctcttgcctgccactgtgtgaatcggcgacggagcactgcacctgcctccagccgccggctgtgcagtcctgggtcctcctttctgagggcccgtgtaaatatgtacatttctcaggctaggccagcaggggctgcccgagtctgtttttcatgcgatgacacttgtacaattaattatcttttcaaaggtacttggataataatgaaataaaactgtttttgaacctgcaaaaaaaaaaaaaaa

SEQ ID NO: 2 - DNA, 3567 bp, Homo sapiens
tctggctcgggctctggctcgggaggcgatcgtggccgctgccgccgccagagccggccggagtcggcccggagccgcgtccacgcggagcgggcgggtgggagctggcggctgggcccctgcggcgcccccgcggcctgccccgcgtccgcctccgggccgccgagccgcagccgccgagatgggggccgccccgggccgcgcccccgccgggtcccgcccgccgcgctgccgctgagcgcatgggcccggaccgcgccgcgccgctccgggagccgggcccggggtcccgccaccaccgcgcgcgggacagattgattcactttggagctgtaagtactgatgtattagggtgcagcgctcattgttccttgacgcagagtcccaaaatgaatatccaagagcagggtttccccttggacctcggagcaagtttcaccgaagatgctccccgacccccagtgcctggtgaggagggagaactggtgtccacagacccgaggcccgccagctacagtttctgctccgggaaaggtgttggcattaaaggtgagacttcgacggccactccgaggcgctcggatctggacctggggtatgagcctgagggcagtgcctcccccaccccaccatacttgaagtgggctgagtcactgcattccctgctggatgaccaagatgggataagcctgttcaggactttcctgaagcaggagggctgtgccgacttgctggacttctggtttgcctgcactggcttcaggaagctggagccctgtgactcgaacgaggagaagaggctgaagctggcgagagccatctaccgaaagtacattcttgataacaatggcatcgtgtcccggcagaccaagccagccaccaagagcttcataaagggctgcatcatgaagcagctgatcgatcctgccatgtttgaccaggcccagaccgaaatccaggccactatggaggaaaacacctatccctccttccttaagtctgatatttatttggaatatacgaggacaggctcggagagccccaaagtctgtagtgaccagagctctgggtcagggacagggaagggcatatctggatacctgccgaccttaaatgaagatgaggaatggaagtgtgaccaggacatggatgaggacgatggcagagacgctgctccccccggaagactccctcagaagctgctcctggagacagctgccccgagggtctcctccagtagacggtacagcgaaggcagagagttcaggtatggatcctggcgggagccagtcaacccctattatgtcaatgccggctatgccctggccccagccaccagtgccaacgacagcgagcagcagagcctgtccagcgatgcagacaccctgtccctcacggacagcagcgtggatgggatccccccatacaggatccgtaagcagcaccgcagggagatgcaggagagcgtgcaggtcaatgggcgggtgcccctacctcacattccccgcacgtaccgggtgccgaaggaggtccgcgtggagcctcagaagttcgcggaggagctcatccaccgcctggaggctgtgcagcgcacgcgggaggccgaggagaagctggaggagcggctgaagcgcgtgcgcatggaggaggaaggtgaggacggcgatccatcgtcagggcccccagggccgtgtcacaagctgcctcccgcccccgcttggcaccacttcccgccccgctgtgtggacatgggctgtgccgggctccgggatgcacacgaggagaaccctgagagcatcctggacgagcacgtacagcgtgtgctgaggacacctggccgccagtcgcctgggcctggccatcgctccccggacagtgggcacgtggccaagatgccagtggcactggggggtgccgcctcggggcacgggaagcacgtacccaagtcaggggcgaagctggacgcggccggcctgcaccaccaccgacacgtccaccaccacgtccaccacagcacagcccggcccaaggagcaggtggaggccgaggccacccgcagggcccagagcagcttcgcctggggcctggaaccacacagccatggggcaaggtcccgaggctactcagagagtgttggcgctgcccccaacgccagtgatggcctcgcccacagtgggaaggtgggcgtggcgtgcaaaagaaatgccaagaaggctgagtcggggaagagcgccagcaccgaggtgccaggtgcctcggaggatgcggagaagaaccagaaaatcatgcagtggatcattgagggggaaaaggagatcagcaggcaccgcaggaccggccacgggtcttcggggacgaggaagccacagccccatgagaactccagacccttgtcccttgagcacccctgggccggccctcagctccggacctccgtgcagccctcccacctcttcatccaagaccccaccatgccaccccacccagctcccaaccccctaacccagctggaggaggcgcgccgacgtctggaggaggaagaaaagagagccagccgagcaccctccaagcagaggacaagatcgcagaggaaggtgggcggcgggagtgcccagccgtgtgacagcatcgttgtggcgtactacttctgcggggaacccatcccctaccgcaccctggtgaggggccgcgctgtcaccctgggccagttcaaggagctgctgaccaaaaagggcagctacagatactacttcaagaaagtgagcgacgagtttgactgtggggtggtgtttgaggaggttcgagaggacgaggccgtcctgcccgtctttgaggagaagatcatcggcaaagtggagaaggtggactgataggctggtgggctggccgctgtgccaggcgaggcccttggcgggcacgggtgtcacggccaggcagatgacctcgtactcaggagcccgatggggaacagtgttgggtgtaccacccatccctgtggtctacccgtgtctagaggcaggtagggggtccctccaagtggtccacaagcttctgtcctgcccccaaggaggcagcctggaccactcctcatagcaatacttggagggcccagcccaagtgaggcagccgaggtccctgctgccagcttcaggtgacccccccccatcccccggcacctcccttgggcacgtgtgctgggatctactttccctctgggatttgcccacgtacccaggtctgggtggggcccaggcccggatgcagaggcctgcagggcctctgtcaattgtacgcgccaccgagtgccttcaacacagcttgtctcttgcctgccactgtgtgaatcggcgacggagcactgcacctgcctccagccgccggctgtgcagtcctgggtcctcctttctgagggcccgtgtaaatatgtacatttctcaggctaggccagcaggggctgcccgagtctgtttttcatgcgatgacacttgtacaattaattatcttttcaaaggtacttggataataatgaaataaaactgtttttgaacctgcaaaaaaaaaaaaaaa

SEQ ID NO: 3 - protein, 862 aa, Homo sapiens
MNIQEQGFPLDLGASFTEDAPRPPVPGEEGELVSTDPRPASYSFCSGKGVGIKGETSTATPRRSDLDLGYEPEGSASPTPPYLKWAESLHSLLDDQDGISLFRTFLKQEGCADLLDFWFACTGFRKLEPCDSNEEKRLKLARAIYRKYILDNNGIVSRQTKPATKSFIKGCIMKQLIDPAMFDQAQTEIQATMEENTYPSFLKSDIYLEYTRTGSESPKVCSDQSSGSGTGKGISGYLPTLNEDEEWKCDQDMDEDDGRDAAPPGRLPQKLLLETAAPRVSSSRRYSEGREFRYGSWREPVNPYYVNAGYALAPATSANDSEQQSLSSDADTLSLTDSSVDGIPPYRIRKQHRREMQESVQVNGRVPLPHIPRTYRVPKEVRVEPQKFAEELIHRLEAVQRTREAEEKLEERLKRVRMEEEGEDGDPSSGPPGPCHKLPPAPAWHHFPPRCVDMGCAGLRDAHEENPESILDEHVQRVLRTPGRQSPGPGHRSPDSGHVAKMPVALGGAASGHGKHVPKSGAKLDAAGLHHHRHVHHHVHHSTARPKEQVEAEATRRAQSSFAWGLEPHSHGARSRGYSESVGAAPNASDGLAHSGKVGVACKRNAKKAESGKSASTEVPGASEDAEKNQKIMQWIIEGEKEISRHRRTGHGSSGTRKPQPHENSRPLSLEHPWAGPQLRTSVQPSHLFIQDPTMPPHPAPNPLTQLEEARRRLEEEEKRASRAPSKQRYVQEVMRRGRACVRPACAPVLHVVPAVSDMELSETETRSQRKVGGGSAQPCDSIVVAYYFCGEPIPYRTLVRGRAVTLGQFKELLTKKGSYRYYFKKVSDEFDCGVVFEEVREDEAVLPVFEEKIIGKVEKVD

SEQ ID NO: 4 - protein, 826 aa, Homo sapiens
MNIQEQGFPLDLGASFTEDAPRPPVPGEEGELVSTDPRPASYSFCSGKGVGIKGETSTATPRRSDLDLGYEPEGSASPTPPYLKWAESLHSLLDDQDGISLFRTFLKQEGCADLLDFWFACTGFRKLEPCDSNEEKRLKLARAIYRKYILDNNGIVSRQTKPATKSFIKGCIMKQLIDPAMFDQAQTEIQATMEENTYPSFLKSDIYLEYTRTGSESPKVCSDQSSGSGTGKGISGYLPTLNEDEEWKCDQDMDEDDGRDAAPPGRLPQKLLLETAAPRVSSSRRYSEGREFRYGSWREPVNPYYVNAGYALAPATSANDSEQQSLSSDADTLSLTDSSVDGIPPYRIRKQHRREMQESVQVNGRVPLPHIPRTYRVPKEVRVEPQKFAEELIHRLEAVQRTREAEEKLEERLKRVRMEEEGEDGDPSSGPPGPCHKLPPAPAWHHFPPRCVDMGCAGLRDAHEENPESILDEHVQRVLRTPGRQSPGPGHRSPDSGHVAKMPVALGGAASGHGKHVPKSGAKLDAAGLHHHRHVHHHVHHSTARPKEQVEAEATRRAQSSFAWGLEPHSHGARSRGYSESVGAAPNASDGLAHSGKVGVACKRNAKKAESGKSASTEVPGASEDAEKNQKIMQWIIEGEKEISRHRRTGHGSSGTRKPQPHENSRPLSLEHPWAGPQLRTSVQPSHLFIQDPTMPPHPAPNPLTQLEEARRRLEEEEKRASRAPSKQRTRSQRKVGGGSAQPCDSIVVAYYFCGEPIPYRTLVRGRAVTLGQFKELLTKKGSYRYYFKKVSDEFDCGVVFEEVREDEAVLPVFEEKIIGKVEKVD

SEQ ID NO: 5 - DNA, 10 bp, Artificial Sequence
  Description of Artificial Sequence Synthetic oligonucleotide
agatcaaagg

SEQ ID NO: 6 - DNA, 21 bp, Artificial Sequence
  Description of Artificial Sequence Synthetic oligonucleotide
ggauguucacaaccgaauutt

SEQ ID NO: 7 - DNA, 21 bp, Artificial Sequence
  Description of Artificial Sequence Synthetic oligonucleotide
gaaaguacauucuugauaatt

SEQ ID NO: 8 - DNA, 21 bp, Artificial Sequence
  Description of Artificial Sequence Synthetic oligonucleotide
ggauaccugccgaccuuaatt

SEQ ID NO: 9 - DNA, 21 bp, Artificial Sequence
  Description of Artificial Sequence Synthetic oligonucleotide
guauuacagcuacucgaaatt

SEQ ID NO: 10 - DNA, 25 bp, Artificial Sequence
  Description of Artificial Sequence Synthetic oligonucleotide
ccuuucagugcuaccttcatctctt

SEQ ID NO: 11 - DNA, 21 bp, Artificial Sequence
  Description of Artificial Sequence Synthetic oligonucleotide
ggaacagucuacaagggaatt

SEQ ID NO: 12 - DNA, 21 bp, Artificial Sequence
  Description of Artificial Sequence Synthetic oligonucleotide
gcuuucagucagauguauatt

SEQ ID NO: 13 - RNA, 25 nt, Artificial Sequence
  Description of Artificial Sequence Synthetic oligonucleotide
gaugacuggacaguuaccuuauuca

SEQ ID NO: 14 - DNA, 21 bp, Artificial Sequence
  Description of Artificial Sequence Synthetic oligonucleotide
gctacagagaaaucucgautt

(19) United States
(12) Patent Application Publication
(10) Pub. No.: US 2012/0059021 A1
(43) Pub. Date: Mar. 8, 2012

(54) COMPOSITIONS AND METHODS FOR TREATING CANCER AND METHODS FOR PREDICTING A RESPONSE TO SUCH TREATMENTS

(75) Inventors: Travis L. BIECHELE, Seattle, WA (US); Andy J. CHIEN, Kirkland, WA (US); Randall T. MOON, Kenmore, WA (US); Rima KULIKAUSKAS, Seattle, WA (US); Rachel TORONI, Auburn, WA (US)

(73) Assignee: UNIVERSITY OF WASHINGTON, Seattle, WA (US)

(21) Appl. No.: 13/224,121

(22) Filed: Sep. 1, 2011

Related U.S. Application Data
(63) Continuation-in-part of application No. 13/128,673, filed on Aug. 3, 2011.
(60) Provisional application No. 61/379,359, filed on Sep. 1, 2010.
(60) Provisional application No. 61/113,461, filed on Nov. 11, 2008.

(51) Int. Cl. A61K 31/506; A61K 31/4184; A61K 31/277; C12Q 1/68; A61P 35/00; G01N 33/566; C40B 30/04; A61K 31/437; A61K 31/4439

(52) U.S. Cl. 514/272; 514/300; 514/394; 514/523; 514/341; 436/501; 435/7.92; 435/7.21; 506/9; 435/6.12

(57) ABSTRACT

The present disclosure relates to the regulation and function of the Wnt/β-catenin signaling pathway and the ERK signaling pathway. The disclosure provides methods of treatment for melanoma by administering both an inhibitor of ERK signaling and an activator of Wnt/β-catenin signaling. These methods may be used alone or in combination with other strategies targeting melanoma cell survival. The disclosure also provides diagnostic methods for predicting a patient's clinical response to inhibitors of ERK signaling.

This application claims benefit under 35 U.S.C. §119(e) of U.S. Provisional Patent Application Ser. No. 61/379,359 filed Sep. 1, 2010 and is a Continuation-in-Part of U.S. patent application Ser. No. 13/128,673 filed Aug. 3, 2011 and which claims benefit as applicable under 35 U.S.C. Sections 120, 121 or 365(c) and which is a 371 National Phase Entry Application of International Application No. PCT/US2009/063858 filed Nov. 10, 2009, which designates the U.S., and which claims benefit under 35 U.S.C. §119(e) of U.S. Provisional Patent Application Ser. No. 61/113,461 filed on Nov. 11, 2008, the contents of which are hereby incorporated by reference in their entirety.

### GOVERNMENT SUPPORT

This invention was made with government support under grant numbers 1K08128565-01 awarded by National Institutes of Health (NIH), T32AR056969 awarded by the NIH and the National Institutes of Arthritis and Muscoloskeletal and Skin (NIAMS), and K08CA128565 awarded by the NIH and the National Cancer Institute (NCI). The government has certain rights in this invention.

### FIELD OF THE INVENTION

The present disclosure related to biological signal transduction and the treatment of cancer.

### BACKGROUND

The majority of both benign nevi and cutaneous melanomas harbor activating mutations in the BRAF oncogene, with BRAF^{V600E} representing the most common of these mutations (1). Mutation of BRAF in this context leads to activation of the downstream MAPK signaling cascade that includes MEK and ERK (i.e. the ERK signaling pathway). The recent development of small molecule compounds designed to specifically target BRAF^{V600E}, including PLX4720 (2), PLX4032/RG7204 (3,4), and GSK2118436 (5) has led to subsequent clinical trials that demonstrated an unprecedented 50-70% objective clinical response rate in patients with BRAF^{V600E} tumors (5-7). Despite these promising results, a significant percentage of patients with BRAF^{V600E} tumors do not meet criteria for an objective clinical response to targeted BRAF^{V600E} inhibition, and the majority of patients who initially respond to BRAF^{V600E} inhibitors eventually develop resistant tumors and progressive disease. Furthermore, the lack of response seen in some patients with BRAF^{V600E} tumors implicates unidentified regulatory mechanisms as important determinants of therapeutic response.

### SUMMARY

Embodiments of the invention described herein are based upon the discovery that BRAF, a component of the ERK signaling pathway, is a major regulator of Wnt/β-catenin signaling in melanoma cells harboring the activating BRAF^{V600E} mutation. In half of the BRAF^{V600E}-mutant cell lines tested, simultaneous activation of Wnt/β-catenin signaling in the presence of inhibition of ERK signaling results in synergistic apoptosis.

Only minimal levels of apoptosis were seen by individual treatment with either Wnt/β-catenin signaling activation or ERK signaling inhibition alone. Susceptibility to apoptosis directly correlated with observed Wnt/β-catenin signaling enhancement upon inhibition of ERK signaling

Upon Wnt/β-catenin activation, inhibition of ERK signaling leads to decreased AXIN1 protein levels and decreased phosphorylation of β-catenin. The extent of decreased AXIN1 predicts susceptibility of cells to both Wnt/β-catenin activation and to Wnt/β-catenin-driven apoptosis upon ERK inhibition. Importantly, knockdown of AXIN1 confers apoptosis susceptibility to resistant cell lines upon inhibition of ERK signaling and activation of Wnt/β-catenin signaling.

In one aspect described herein is a method of treating melanoma in a subject, the method comprising, 1) administering a therapeutically effective amount of an inhibitor of ERK signaling; and 2) administering a therapeutically effective amount of an activator of the Wnt/β-catenin signaling pathway.

In some embodiments, the subject is a human.

In some embodiments, the method further comprises administering to the subject a therapeutically effective amount of a PI3K inhibitor.

In some embodiments, the inhibitor of ERK signaling is selected from the group consisting of inhibitors of ERK1/2, inhibitors of BRAF, inhibitors of a BRAF mutant, inhibitors of BRAF^{V600E} and inhibitors of MEK. In some embodiments, the inhibitor of a component of ERK signaling is a small molecule inhibitor. In some embodiments, the inhibitor of a component of ERK signaling is selected from the group consisting of PLX4720, PLX4032 (vemurafenib), AZD6244, GSK2118436 and U0126.

In some embodiments, the activator of the Wnt/β-catenin signaling pathway is a GSK3β inhibitor. In some embodiments, the GSK3β inhibitor is selected from the group consisting of CHIR99021 and CHIR-837. In some embodiments the activator of the Wnt/β-catenin signaling pathway is a Wnt ligand.

In some embodiments, the administration of the inhibitor of ERK signaling and the activator of Wnt/β-catenin signaling pathway synergistically increase tumor cell apoptosis.

Another aspect described herein is a method of predicting the response of a subject in need of treatment for melanoma to treatment with an inhibitor of ERK signaling and optionally an activator of Wnt/β-catenin comprising, 1) determining an amount of an AXIN1 protein in a biological sample obtained from the subject; and 2) comparing the amount to a reference value; wherein an amount of an AXIN1 protein in the biological sample which is equal to or greater than the reference value indicates that the subject will be less likely to respond to the inhibitor and optionally the activator; and wherein an amount of an AXIN1 protein in the biological sample which is less than the reference value indicates that the subject will be more likely to respond to the inhibitor and optionally the activator.

In some embodiments of this aspect, the biological sample is obtained after the subject is administered a dose of an inhibitor of ERK signaling and the reference value is an amount of AXIN1 protein determined in a biological sample obtained from said subject prior to administering said inhibitor of ERK signaling.

In some embodiments of the second aspect, the method further comprises administering an inhibitor of ERK signaling and an activator of Wnt/β-catenin signaling to the subject when the level of the AXIN1 gene product is less than the reference value.

Another aspect described herein relates to a method of predicting the response of a subject in need of treatment for melanoma to treatment with an inhibitor of ERK signaling and optionally an activator of Wnt/β-catenin signaling, the method comprising, 1) determining an amount of a nuclear β-catenin marker in a biological sample obtained from the subject; and 2) comparing the amount to a reference value; wherein an amount of a nuclear β-catenin marker in the biological sample which is greater than the reference value indicates that the subject will be more likely to respond to the inhibitor and optionally the activator; and wherein an amount of a nuclear β-catenin marker in the biological sample which is less than the reference value indicates that the subject will be less likely to respond to the inhibitor and optionally the activator.

Another aspect described herein relates to a method of treating melanoma in a subject, the method comprising, 1) determining an amount of a nuclear β-catenin marker in a biological sample obtained from the subject; 2) comparing the amount to a reference value; and 3) administering an inhibitor of ERK signaling and optionally an activator of Wnt/β-catenin when the amount of a nuclear β-catenin marker in the biological sample is greater than the reference value, wherein said melanoma is more sensitive to treatment with the inhibitor of ERK signaling than a melanoma with an amount of a marker of nuclear β-catenin that is less than the reference value.

Another aspect described herein relates to a method of treating melanoma in a subject unresponsive to treatment with an inhibitor of ERK signaling and an activator of Wnt/β-catenin signaling, the method comprising, 1) administering a therapeutically effective amount of an inhibitor of AXIN1, 2) administering a therapeutically effective amount of an inhibitor of ERK signaling, and 3) administering a therapeutically effective amount of an activator of the Wnt/β-catenin signaling pathway; thereby treating melanoma in a subject unresponsive to treatment with an inhibitor of ERK signaling and an activator of Wnt/β-catenin signaling.

### DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1E depict the results of a kinome-based siRNA screen demonstrating that BRAF/MAPK signaling negatively regulates Wnt/β-catenin signaling in melanoma cells. FIG. 1A is a scatter plot of a kinome-based siRNA screen in human A375 melanoma cells stably expressing the β-catenin-activated reporter (BAR) driving expression of firefly luciferase, with each dot representing a known or predicted kinase. Cells transfected with siRNA were subsequently treated with WNT3A conditioned media (CM), and luciferase activity was normalized to cell number as measured by a resazurine. Red- and green-dotted lines represent two mean absolute deviations (MADs) above and below the mean, respectively. FIG. 1B is an isobologram analysis of the targeted BRAF inhibitor PLX4720 and WNT3A CM on BAR activity in A375 melanoma cells. FIG. 1C depicts immunoblot results demonstrating that dose-dependent inhibition of dual-phosphorylated ERK1/2 (ppERK1/2) by PLX4720 coincided with reduced phosphorylation of β-catenin at sites that normally target it for proteasomal degradation (Ser33/Ser37/Thr41), as well as reduced auto-activation of GSK3β through phosphorylation at Tyr216. FIG. 1D is a graph demonstrating that two distinct MEK inhibitors, U0126 and AZD6244, both enhanced Wnt/β-catenin activation in a dose-dependent manner. Columns and error bars represent the mean and standard deviation, respectively, of three biologic replicates. FIG. 1E depicts immunoblot results demonstrating that the inhibition of ppERK1/2 by U0126 and AZD6244 occurs in a dose-dependent manner corresponding to the activation of BAR by these drugs in FIG. 1D. In FIGS. 1B-1D, data are representative of at least three independent experiments.

FIGS. 2A-2C depict results demonstrating that Wnt/β-catenin activation cooperates with targeted inhibition of mutant BRAF to inhibit tumor growth in vivo and in vitro. FIG. 2A is a graph of the growth of human A375 melanoma cells expressing either GFP or WNT3A(iresGFP). Cells were grown as xenografts in NSG mice treated with either vehicle or 50 mg/kg PLX4720 after tumors had reached an initial size of 100 mm³. Note that tumors expressing WNT3A took longer to achieve this initial size than tumors expressing GFP. For each treatment arm, the means and SEM are shown for five individual mice. FIG. 2B depicts human A375 melanoma cells expressing either GFP or WNT3A(iresGFP) that were grown as spheroids in a three-dimensional collagen matrix, then treated with either DMSO or 2 μM PLX4720 for 72 hours prior to imaging. Representative spheroids of greater than forty spheroids per treatment are shown in these light micrographs. FIG. 2C depicts an isobologram analysis of PLX4720 and WNT3A CM on the viability of A375 melanoma cells as measured by CellTiter-Glo. A375 melanoma cells were treated with the indicated combinations of WNT3A CM and PLX4720 concentrations for 48 hours. Data are representative of at least three independent experiments with each data point assayed in triplicate.

FIGS. 3A-3D demonstrate that Wnt/β-catenin activation synergistically enhances apoptosis with BRAF inhibition. FIG. 3A depicts photomicroscopy of TUNEL assays which revealed the presence of apoptotic cells upon treatment with WNT3A CM and 2 μM PLX4720, with minimal TUNEL-positivity seen with DMSO vehicle, PLX4720 or WNT3A CM individually in A375 cells. DAPI staining of nuclei was used to visualize all cells within the field. FIG. 3B depicts spheroids generated from A375 cells expressing either GFP or WNT3A(iresGFP), grown in a three-dimensional collagen matrix and treated with either DMSO or 2 μM PLX4720 for 24 hours. Simultaneously, GFP was used to image all spheroids while EtBr staining was used to identify dead cells. Representative spheroids of greater than forty spheroids for each condition are shown in these panels. FIG. 3C depicts the results of a flow cytometry-based assay for apoptosis based on detection of cleaved caspase-3 showed minimal effects of DMSO vehicle, 2 μM PLX4720, or WNT3A CM individually in A375 cells. The combination of WNT3A CM and PLX4720 led to a marked increase in the percentage of cells positive for cleaved caspase-3 (indicated by blue histogram peaks), consistent with the results seen in FIG. 3A with the TUNEL assay. Cells were treated for 24 hours with the indicated conditions before analysis. Red peaks on the histogram indicate the distribution of cells that were negative for caspase-3 staining, while blue numbers indicate the percentage of caspase-3 positive cells in these representative panels. FIG. 3D depicts immunoblot analysis of proteins involved in apoptosis shows that WNT3A enhances PLX4720-mediated expression of BimL and BimS (lane 3 compared to land 4 and lane 7 compared to lane 8). A375 cells were treated for 24 hours with the indicated conditions in combination with DMSO vehicle or the pan-caspase inhibitor, Z-VAD-FMK. 2 μM PLX4720 was used in this experiment. In FIGS. 3A, 3C and 3D, data are representative of at least three independent experiments.

FIGS. 4A-4B demonstrate that apoptosis mediated by Wnt/β-catenin signaling and BRAF inhibition requires β-catenin. FIG. 4A depicts an immunoblot-based assay for cleaved caspase-3 which confirmed enhanced apoptosis in A375 cells treated with WNT3A CM and 2 μM PLX4720 in the presence of control siRNA (lane 4). Apoptosis was completely inhibited by pretreatment with siRNA targeting β-catenin (CTNNB1) (lane 8). Note that apoptosis seen with PLX4720 alone (lane 3) was completely blocked by β-catenin knockdown (lane 7). Cells were transfected with siRNAs, and at 48 hours post-transfection cells were then treated with the indicated conditions for 48 hours. FIG. 4B depicts immunoblot results demonstrating that CHIR99021, a small molecule GSK3β inhibitor that activates Wnt/β-catenin signaling, also synergizes with PLX4720 to enhance apoptosis in A375 melanoma cells. Cells were transfected with siRNAs, and at 48 hours post-transfection cells were then treated with the indicated combinations of 5 μM CHIR99021 and 2 μM PLX4720 for 36 hours. Inhibition of GSK3β was confirmed by loss of the activating auto-phosphorylation at Tyr216. This induced apoptosis (lanes 3 and 4) is completely inhibited by siRNA knockdown of β-catenin (lanes 3 and 4 compared to lanes 7 and 8). Data are representative of at least three independent experiments.

FIGS. 5A-5D demonstrate that regulation of Wnt/β-catenin signaling and AXIN1 by the ERK signaling pathway predicts response to apoptosis. FIG. 5A is a graph depicting synergistic enhancement of Wnt/β-catenin signaling by BRAF^{V600E} inhibition which was examined in six melanoma lines harboring BRAF^{V600E} mutations along with human epidermal melanocytes (HEM). Cells were treated with control or WNT3A CM and either DMSO vehicle or 2 μM PLX4720 for 24 hours. Activation of Wnt/β-catenin signaling was confirmed by qPCR measurements of the endogenous target gene AXIN2. In A375, MEL624, and COLO829, treatment with PLX4720 enhanced WNT3A-mediated increases in AXIN2 transcript levels. In contrast, A2058, SKMEL28, SKMEL5, and HEMs did not exhibit any increase in AXIN2 transcript levels with PLX4720. Columns and error bars represent the mean and standard deviation, respectively, of three biologic replicates. FIG. 5B depicts flow cytometry detection of active caspase-3 used to measure apoptosis in several melanoma cell lines and HEM treated with the indicated conditions. Enhanced apoptosis following 24 hour treatment with the combination of WNT3A and 2 μM PLX4720 was seen in the same cell lines exhibiting enhanced Wnt/β-catenin signaling under these conditions. Columns and error bars represent the mean and standard deviation, respectively, of three biologic replicates. FIG. 5C depicts the results of immunoblots of melanoma cell lines treated with WNT3A CM in either the absence or presence of 2 μM PLX4720 for 24 hours. In A375, MEL624 and COLO829, the addition of PLX4720 leads to decreased steady-state levels of AXIN1. FIG. 4D depicts a graph in which an immunoblot of AXIN1 from FIG. 5C combined with immunoblots from two additional independent replicates were quantified by pixel intensity. Addition of PLX4720 and WNT3A significantly reduced steady-state AXIN1 levels as compared to WNT3A alone in A375, MEL624, and COLO829 cells. In FIGS. 5A-5C data are representative of at least three independent experiments.

FIGS. 6A-6F demonstrate that AXIN1 levels regulate apoptosis mediated by BRAF inhibition. FIG. 6A depicts immunoblot results of a time-course of A375 cells in which the temporal relationship between decreases in steady-state AXIN levels and apoptosis was analyzed. Steady-state AXIN1 levels decrease within hours after treatment with WNT3A and PLX4720. By contrast, apoptosis as measured by cleaved caspase-3 is not detected until 16 hours after treatment. FIG. 6B depicts immunoblot results demonstrating that the decreased steady-state level of AXIN1 in the context of PLX4720 and WNT3A is not dependent on caspase activation. A375 cells were treated for 24 hours with the indicated combinations of WNT3A and 2 μM PLX4720 in combination with DMSO vehicle or the pan caspase inhibitor, Z-VAD-FMK. Decreased steady-state levels of phosphorylated Ser33/Ser37/Thr41 on β-catenin (pCTNNB1) in the context of PLX4720 and WNT3A is also not dependent on caspase activation (FIG. 12B). FIG. 6C is a graph demonstrating that PLX4720 enhancement of Wnt/β-catenin signaling is not dependent on caspase activation. A375 cells containing the BAR reporter were treated for 24 hours with the indicated combinations of WNT3A and 2 μM PLX4720 in combination with DMSO vehicle or the pan-caspase inhibitor, Z-VAD-FMK. Columns and error bars represent the mean and standard deviation, respectively, of three biologic replicates. FIG. 6D depicts flow cytometry results. In SKMEL28 cells transfected with control siRNA, minimal changes in cleaved caspase-3 were seen by flow cytometry, even upon WNT3A and 2 μM PLX4720 treatment (left panels). In cells transfected with AXIN1/2 siRNA, treatment with 2 μM PLX4720 led to a significant increase in cleaved caspase-3 (right panels). Numbers indicate the percentage of caspase-3 positive cells in these representative panels. Similar results were seen in A2058 and SKMEL5 melanoma cells (Table 3). FIG. 6E is a graph demonstrating that individual knockdown of AXIN1, but not AXIN2, by siRNA confers increased apoptosis in SKMEL28 cells. SKMEL28 cells were transfected with control siRNA or siRNAs targeting AXIN1, AXIN2 or both AXIN/2. Cells were then treated with DMSO vehicle or 2 uM PLX4720 and cleaved caspase3 was measured by flow cytometry. Columns and error bars represent the mean and standard deviation, respectively, of three biologic replicates. FIG. 6F depicts immunoblot results demonstrating that knockdown of AXIN1 by siRNA sensitizes SKMEL28 cells to PLX4720-induced apoptosis. Immunoblots of SKMEL28 cells transfected with either control or two non-overlapping independent siRNAs targeting AXIN1 show that PARP cleavage was strongly induced following 24 hour treatment of 2 uM PLX4720 in both AXIN1 siRNA treated samples (lanes 4 and 6) when compared to control siRNA (lane 2). In FIGS. 6A-6F, data are representative of at least three independent experiments.

FIGS. 7A-7B demonstrate that BRAF and other members of the MAPK/ERK family are identified as regulators of Wnt/β-catenin signaling in melanoma cells. FIG. 7A demonstrates that the distribution of siRNA screen data is not biased with respect to cell viability as measured by resazurine. Results of BAR activation in the kinome siRNA library screen at 1.8 nM final siRNA concentration in A375 melanoma cells are plotted against cell viability as measured by resazurine (x-axis), with each point representing a siRNA pool targeting a single gene product. The red line is the best fit line and indicates no significant correlation between BAR reporter activity and cell viability. FIG. 7B is a heatmap of the siRNA screen results. The siRNA screen was performed over four separate final concentrations of siRNA (9.5 nM, 1.9 nM, 0.38 nM, and 0.08 nM), and this heatmap shows the dose-dependent activation of BAR seen with targeted knockdown of multiple members of the MAPK/ERK cascade, further implicating this pathway as a regulator of Wnt/β-catenin signaling in melanoma cells. Also shown is a heatmap of included gene targets encoding proteins previously published to interact with the BRAF signaling complex, including RAF1 (C-RAF) and the scaffolding protein KSR1. Of note, no effect is seen with C-RAF knockdown in this screen. Interestingly, knockdown of KSR1 leads to a dose-dependent inhibition of Wnt/β-catenin signaling, indicating that KSR1 is required for Wnt/β-catenin signaling in this cell context.

FIGS. 8A-8D demonstrate that BRAF is a negative regulator of Wnt/β-catenin signaling and inhibition of BRAF decreases steady-state phosphorylation of β-catenin at Ser33/Ser37/Thr41 in melanoma cells. FIG. 8A is a graph of the results of different siRNAs and their effect on Wnt activation. The negative regulation of Wnt/β-catenin signaling by BRAF in A375 cells was further verified by individually testing five non-overlapping siRNAs targeting BRAF, including a previously published sequence (MUT-A) designed to specifically target the BRAF^{V600E} mutation (24). A375 cells stably expressing the BAR reporter were transfected with the indicated siRNAs. 48 hours post transfection, cells were treated with control or WNT3A conditioned media for 24 hours and BAR reporter activity was measured. Knockdown of BRAF in the presence of WNT3A results in synergistic activation of Wnt/β-catenin signaling that is comparable to siRNA-mediated knockdown of AXIN, a central conserved component of the Wnt/β-catenin pathway. FIG. 8B depicts Western blot analysis showing that all five BRAF-directed siRNAs reduced the expression of BRAF protein and robustly inhibited ppERK1/2 in A375 cells. It was rather surprising that BRAF protein levels were only modestly reduced given that ppERK1/2 was almost undetectable. FIG. 8C is a graph of quantitative real-time PCR which was used to confirm knockdown of BRAF transcripts by the BRAF siRNAs in A375 cells. Each BRAF siRNA knocked down nearly 90% of BRAF transcripts compared to control siRNA. Data are averages of three independent siRNA transfections and error bars represent standard deviations. FIG. 8D demonstrates that PLX4720 strongly decreases the steady-state phosphorylation of β-catenin at Ser33/Ser37/Thr41. A375 melanoma cells were treated with the indicated conditions for 24 hours. Cell lysates were fractionated into cytosolic and nuclear fractions as described in the methods and samples were separated by PAGE, transferred to nitrocellulose, and probed with the indicated antibodies. In FIGS. 8A-8D, data are representative of at least three independent experiments.

FIGS. 9A-9C depict isobologram analysis of WNT3A and PLX4720 showing a synergistic activation of Wnt/β-catenin signaling in melanoma cells. A375 cells stably expressing BAR-luciferase were treated with combinations of WNT3A and PLX4720 across a range of doses. BAR activity was measured with a standard luciferase assay and normalized to constitutively expressed renilla luciferase activity. FIG. 9A depicts dose-response curves for PLX4720, WNT3A, and the combination of PLX4720+WNT3A (at fixed ratio doses). Note that PLX4720 does not lead to any measurable activation of BAR on its own. FIG. 9B depicts a median-effect plot which demonstrates that the combination of PLX4720+WNT3A decreases the dosage of drugs required for median effect, as indicated by the x-intercept. FIG. 9C depicts a plot demonstrating that with increasing activation of BAR, synergy between WNT3A and PLX4720 using fixed ratio doses is indicated by decreasing combination indices less than 1. The solid red line represents the calculated curve, with 1.96 S.D. (95% confidence interval) indicated by the dashed lines.

FIGS. 10A-10D demonstrate that activation of Wnt/β-catenin signaling combined with BRAF inhibition acts synergistically to inhibit proliferation of melanoma cells in vivo. FIG. 10A depicts a schematic representation of the capillary-based isoelectric focusing method of detecting levels of ppERK1/2 in fine needle aspirates of xenograft tumors from vehicle or PLX4720 treated mice (top panel). Fine needle aspiration procedures (FNA) were performed on xenograft tumors three days prior to treatment for baseline samples and 2 hours after the initial oral gavage treatment of 50 mg/kg PLX4720. FNAs were analyzed for ppERK1/2 and HSP70 for normalization by capillary-based isoelectric focusing on a Nanopro1000 instrument (Cell Biosciences)(bottom panel). FIG. 10B is a graph demonstrating that xenograft tumors have significantly less ppERK2 following treatment with PLX4720. FNAs of xenograft tumors three days before treatment (baseline) or two hours following the initial treatment of 50 mg/kg PLX4720 were analyzed for ppERK1/2 and normalized to levels of HSP70. Columns and error bars represent the mean and standard deviation, respectively, from three xenograft tumors. FIG. 10C is a graph of the results from an experiment in which xenografts from FIG. 2A were compared at day 23, which was the last day at which all animals had tumor. Bars represent the mean and standard error for each group of tumors, while individual tumors are represented for each condition by gray symbols. The differences were extremely significant by one-way ANOVA with a post-test for linear trend (p<0.0001). FIG. 10D is a graph of results from an experiment in which xenograft tumors from FIG. 2A were sectioned, stained with hematoxylin- and eosin, and analyzed for mitotic cells by microscopy under high-power as described elsewhere herein. Bars represent the mean and standard error for each group of tumors, while gray symbols are used to reflect the average for individual tumors within each group. Differences between the tumors was extremely significant (p<0.0001) by one-way ANOVA with a post-test for linear trend.

FIGS. 11A-11C depict isobologram analysis of WNT3A and PLX4720 which shows a synergistic inhibition of melanoma cell viability. A375 cells were treated with combinations of WNT3A and PLX4720 across a range of doses. FIG. 11A depicts dose-response curves for PLX4720, WNT3A, and the combination of PLX4720+WNT3A (at fixed ratio doses). FIG. 11B depicts a median-effect plot which shows that the combination of PLX4720+WNT3A decreases the dosage of drugs required for median effect, as indicated by the x-intercept. FIG. 11C depicts a graph demonstrating that with increasing growth inhibition, synergy between WNT3A and PLX4720 using fixed ratio doses is indicated by decreasing combination indices less than 1. The solid red line represents he calculated curve, with 1.96 S.D. (95% confidence interval) indicated by the dashed lines.

FIGS. 12A-12B demonstate that activation of Wnt/β-catenin signaling combined with siRNA-mediated knockdown of BRAF promotes apoptosis of melanoma cells. FIG. 12A depicts immunoblot results of an experiment in which human A375 melanoma cells were treated with control siRNA or siRNA targeting BRAF^{V600E} (MUT-A). At 48 hours post transfection, cells were untreated (C) or treated with control (L) or WNT3A (W3) conditioned media for 24 hours. Cells were then harvested and analyzed by Western blot with the indicated antibodies. FIG. 12B depicts immunoblot results from an experiment in which A375 melanoma cells were treated with the indicated conditions for 24 hours. Cell lysates were pre-cleared with concanavalin-A (Con-A) sepharose beads overnight at 4° C. to remove membrane-bound fractions. Cleared lysates were then immunoblotted with the indicated antibodies.

FIGS. 13A-13B demonstrate that BRAF inhibition regulates steady-state protein levels through a proteasome mediated mechanism. FIG. 13A is a graph of AXIN1 levels in A375 cells treated with the indicated conditions for 24 hours, as quantitatively-measured by qRT-PCR. AXIN1 mRNA levels were unchanged compared to vehicle treated cells. A final concentration of 2 μM PLX4720 was used in this experiment. Bars represent averages of three independent biological replicates with error bars representing standard deviations. FIG. 13B depicts immunoblot results from an experiment in which A375 cells were treated with control conditioned media and DMSO (control) or WNT3A CM and 2 μM PLX4720 (W3A+PLX) in combination with DMSO, 10 μM MG132, or 10 μM Chloroquine for 8 hours and subsequently analyzed by immunoblot for effects on AXIN1 and ppERK1/2 levels.

FIGS. 14A-14G demonstrate that BRAF and MEK are negative regulators of Wnt/β-catenin signaling in melanoma cells but not melanocytes. FIGS. 14A-14B demonstrate that inhibition of MEK with U0126 synergizes with WNT3A to activate β-catenin signaling as measured by a Wnt reporter (FIG. 14A) and endogenous AXIN2 transcript levels (FIG. 14B). FIG. 14C depicts a Wnt reporter assay which confirms the screen showing that β-catenin signaling is elevated when BRAF^{V600E} is inhibited by PLX4720 in cells treated with WNT3A. FIG. 14D demonstrates that phosphorylated β-catenin is decreased with increasing doses of PLX4720. FIGS. 14E-14F demonstrates that ERK/MAPK signaling regulates Wnt/β-catenin signaling in the opposite polarity in melanocytes. U0126 treatment (FIG. 14E) inhibits ERK 1/2 phosphorylation in a dose-dependent manner in melanocytes, but there is no effect of BRAF^{V600E} inhibition by PLX4720 (FIG. 14F) on ERK1/2 phosphorylation. FIG. 14G demonstrates that U0126 dose dependently inhibits WNT3A-mediated reporter activity in primary melanocytes and PLX4720 has no effect on Wnt/β-catenin signaling in melanocytes.

FIGS. 15A-15C demonstrate that inhibition of MAPK signaling leads to Wnt-dependent decreases in steady-state levels of AXIN1. FIG. 15A depicts a Western blot showing that AXIN1 levels are reduced by PLX4720 and U0126 in the presence of WNT3A thereby promoting β-catenin signaling. FIG. 15B demonstrates that downstream Wnt-dependent transcriptional responses are not involved in PLX4720 or U0126 regulation of steady state levels of AXIN1. FIG. 15C demonstrates that even in the presence of U0126 or PLX4720, XAV939 can elevate AXIN1 protein levels to above baseline levels.

FIGS. 16A-16C demonstrate that Wnt/β-catenin activation induces apoptosis in the presence of BRAF/MAPK inhibition. FIG. 16A is a graph demonstrating that using a resazurin-based assay for cell viability, there is a decrease in proliferation in human A375 melanoma cells treated with Wnt3A conditioned media compared to cells treated with control L-cell conditioned media. FIG. 16B demonstrates that treatment with PLX4720 further decreases proliferation WNT3A increases apoptosis when BRAF^{V600E} is inhibited as measured by Caspase3 activation. FIG. 16C demonstrates that β-catenin is required for apoptosis in response to WNT3A and PLX4720.

FIGS. 17A-17B demonstrate that siRNAs targeting AXIN1 and AXIN2 sensitize A2058 cells to Caspase3 activation mediated by PLX4720 alone and this response is increased by the combination of WNT3A and PLX4720 as measured using western blot (FIG. 17A) and flow cytometry (FIG. 17B).

FIG. 18 depicts a graph demonstrating that XAV939 is unable to completely reverse the enhancement of Wnt/β-catenin signaling by ERK signaling inhibition.

FIG. 19 depicts a model of ERK/MAPK regulation of Wnt/β-catenin signaling through AXIN and ERK signaling pathway regulation of WNT-mediated apoptosis of melanoma cells.

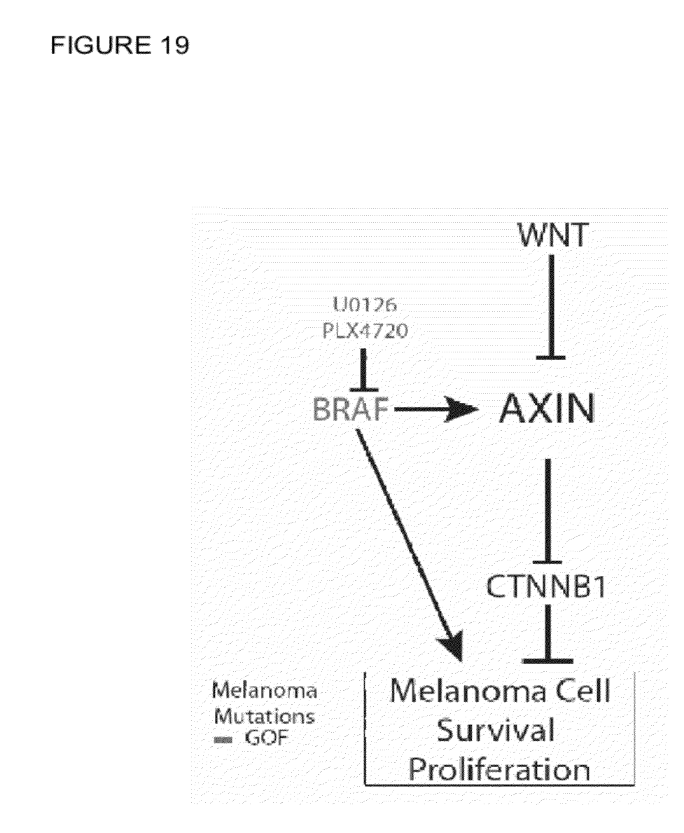

### DETAILED DESCRIPTION

### Definitions

The term “melanoma” as used herein refers to skin cancer derived from melanocytes. There are four major types of melanoma that each constitutes a distinct level of danger owing to their metastatic potential. “Superficial Spreading” is the most common type (70%) of melanoma in Caucasians, usually found on the trunk, upper arms and thighs but it can be anywhere on the body. It begins as a small pigmented, slightly raised asymmetric macule that has irregular borders, and can have many color variations. Superficially Spreading Melanoma typically shows earlier signs of invasiveness than the following two types: “Lentigo” and “Maligna.” Maligna is typically found in elderly people. It is similar to the superficial spreading type and is usually located on the head and neck region. It presents as a flat or slightly elevated mottled dark skin discoloration. It can remain restricted to the epidermis for long periods of time, but it remains potentially invasive (after which it is called Lentigo Maligna Melanoma).

Acral-Lentiginous Melanoma is more commonly found on the palm of hands, soles of feet, and nail beds in African-Americans and Asians. Like the previous two types, it starts out as a superficial spreading tumor that can resemble a wart or fungus. This phase is relatively long before it turns more invasive.

Nodular Melanoma is more often on the trunk, upper arms, and thighs. It is usually diagnosed when it is already invasive. Its color can vary greatly but is most often black. This type of melanoma may ulcerate and present as a non-healing skin ulcer.

Some less common melanoma variants include Desmoplastic malignant Melanoma, which is histologically ill-defined but can involve normal stromal cells to varying degrees in its architecture. It has a high incidence of local recurrence and repeated surgical removal can increase the risk of metastasis.

Giant Melanocytic Nevus is a birthmark (mole) of over 20 cm in diameter. Such moles demand attention because there is a risk of up to 5% that they will develop into melanoma. Amelanotic Malignant Melanoma simply means a tumor without pigment. Lack of dark color (they are usually pink or red) can make it more difficult to spot and recognize. Nevoid Melanoma is a melanoma with a deceptively benign looking histology, resembling normal melanocytes. There are a large number of other variants, even within recognized types mentioned here and they are all considered to be encompassed within the term “melanoma” as used in this application.

As used herein, the term “nuclear β-catenin” refers the form of β-catenin which translocates to and accumulates in the nucleus following activation of Wnt/β-catenin signaling. Upon translocating to the nucleus, the nuclear β-catenin trans-activates expression of target genes. Nuclear β-catenin, is, naturally, found in the nucleus. A cell, tissue and/or tumor with “nuclear β-catenin” can be a cell, tissue and/or tumor with qualitatively visible β-catenin readily detectable, above the background, in its nucleus under appropriate conditions, such as with immunohistochemistry using established antibodies (for example, Sigma-Aldrich Cat #C2206). Based on previous studies, the qualitative detection of β-catenin in the nucleus can be seen either uniformly throughout the tumor, or in small numbers of cells down to a single cell. In either case, the presence of any amount of nuclear β-catenin in tumor cells is presumed to be a surrogate marker of Wnt activation within that cell or tumor, and is not reliant on any specific threshold. β-catenin can be detected in any way known in the art, but at a minimum, nuclear β-catenin is detected by immunohistochemistry using polyclonal rabbit anti-β-catenin antibody (Sigma, Cat# C2206) and goat anti-rabbit Alexa Fluor-568 antibody (Molecular Probes; Eugene, Oreg.) as described herein, for example, in Example 3. Alternatively, the presence of nuclear β-catenin can be determined by detection of downstream gene target activation, e.g., expression of AXIN2 gene expression.

As used herein, the terms “treat,” “treatment,” “treating,” or “amelioration” refer to therapeutic treatments for melanoma, wherein the object is to reverse, alleviate, ameliorate, inhibit, slow down or stop the progression or severity of a symptom or condition associated with melanoma. The term “treating” includes reducing or alleviating at least one adverse effect or symptom of a condition, disease or disorder associated with melanoma. Treatment is generally “effective” if one or more symptoms or clinical markers of melanoma are reduced. Alternatively, treatment is “effective” if the progression of melanoma is reduced or halted. That is, “treatment” includes not just the improvement of symptoms or markers of melanoma, but also a cessation or at least slowing of progress or worsening of symptoms of melanoma that would be expected in the absence of treatment. Beneficial or desired clinical results include, but are not limited to, alleviation of one or more symptom(s), diminishment of extent of the disorder, stabilized (i.e., not worsening) state of the disorder, delay or slowing of disorder progression, amelioration or palliation of the disorder state, and remission (whether partial or total), whether detectable or undetectable. The term “treatment” of a disorder also includes providing relief from one or more symptoms or side-effects of the disorder (including palliative treatment).

The terms “decrease,” “reduce,” “reduced”, “reduction”, “decrease,” “suppress,” “inhibit,” or “inhibition” are all used herein generally to mean a decrease by a statistically significant amount relative to a reference. However, for avoidance of doubt, “reduce,” “reduction” or “decrease” or “inhibit” typically means a decrease by at least about 5%-10% as compared to the absence of the treatment and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% decrease or more, i.e. absent level, as compared to the absence of the treatment, or any decrease between 10-99% as compared to the absence of the treatment.

As used herein, the term “antibody” refers to immunoglobulin molecules and immunologically active portions of immunoglobulin molecules, i.e., molecules that contain an antigen binding site that specifically bind an antigen. The terms also refers to intact antibodies comprised of two immunoglobulin heavy chains and two immunoglobulin light chains as well as a variety of antigen-specific binding other than intact or stereotypical antibodies, including, for example, Fv, scFv, Fab, and F(ab)′2 as well as bifunctional hybrid antibodies (e.g., Lanzavecchia et al., Eur. J. Immunol. 17, 105 (1987)) and single chains (e.g., Huston et al., Proc. Natl. Acad. Sci. U.S.A., 85, 5879-5883 (1988) and Bird et al., Science 242, 423-426 (1988), which are incorporated herein by reference). (See, generally, Hood et al., Immunology, Benjamin, N.Y., 2ND ed. (1984), Harlow and Lane, Antibodies. A Laboratory Manual, Cold Spring Harbor Laboratory (1988) and Hunkapiller and Hood, Nature, 323, 15-16 (1986), which are incorporated herein by reference). The term also includes intrabodies, i.e. antibodies that work within the cell and bind to intracellular protein. Intrabodies can include whole antibodies or antibody binding fragments thereof, e.g. single Fv, Fab and F(ab)′2, etc.

The term “expression” refers to the cellular processes involved in producing RNA and proteins and as appropriate, secreting proteins, including where applicable, but not limited to, for example, transcription, translation, folding, modification and processing. “Expression products” or “gene products” include RNA transcribed from a gene, and polypeptides obtained by translation of mRNA transcribed from a gene. In some embodiments, an expression product is transcribed from a sequence that does not encode a polypeptide, such as a microRNA or RNAi.

The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “complementary” or “complementary base pair” refers to A:T and G:C in DNA and A:U in RNA. Most DNA consists of sequences of nucleotide only four nitrogenous bases: base or base adenine (A), thymine (T), guanine (G), and cytosine (C). Together these bases form the genetic alphabet, and long ordered sequences of them contain, in coded form, much of the information present in genes. Most RNA also consists of sequences of only four bases. However, in RNA, thymine is replaced by uridine (U).

As used herein, the term “proteins” and “polypeptides” are used interchangeably herein to designate a series of amino acid residues connected to the other by peptide bonds between the alpha-amino and carboxy groups of adjacent residues. The terms “protein”, and “polypeptide”, which are used interchangeably herein, refer to a polymer of protein amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles consistent with the disclosure. Typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The term “nucleic acids” used herein refers to polynucleotides such as deoxyribonucleic acid (DNA), and, where appropriate, ribonucleic acid (RNA), polymers thereof in either single- or double-stranded form. Unless specifically limited, the term encompasses nucleic acids containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19:5081 (1991); Ohtsuka, et al., J. Biol. Chem. 260:2605-2608 (1985), and Rossolini, et al., Mol. Cell. Probes 8:91-98 (1994)). The term “nucleic acid” should also be understood to include, as equivalents, derivatives, variants and analogs of either RNA or DNA made from nucleotide analogs, and, single (sense or antisense) and double-stranded polynucleotides.

The term “vector”, as used herein, refers to a nucleic acid construct designed for delivery to a host cell or transfer between different host cells. As used herein, a vector can be viral or non-viral.

As used herein, the term “expression vector” refers to a vector that has the ability to incorporate and express heterologous nucleic acid fragments in a cell. An expression vector may comprise additional elements, for example, the expression vector may have two replication systems, thus allowing it to be maintained in two organisms, for example in human cells for expression and in a prokaryotic host for cloning and amplification.

As used herein, the term “heterologous nucleic acid fragments” refers to nucleic acid sequences that are not naturally occurring in that cell.

As used herein, the term “viral vector” refers to a nucleic acid vector construct that includes at least one element of viral origin and has the capacity to be packaged into a viral vector particle. The viral vector can contain the target gene in place of non-essential viral genes. The vector and/or particle may be utilized for the purpose of transferring any nucleic acids into cells either in vitro or in vivo. Numerous forms of viral vectors are known in the art.

The term “replication incompetent” as used herein means the viral vector cannot further replicate and package its genomes. For example, when the cells of a subject are infected with replication incompetent recombinant adeno-associated virus (rAAV) virions, the heterologous (also known as transgene) gene is expressed in the patient's cells, but, the rAAV is replication defective (e.g., lacks accessory genes that encode essential proteins from packaging the virus) and viral particles cannot be formed in the patient's cells.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated.”

As used herein, the phrase “therapeutically effective amount”, “effective amount” or “effective dose” refers to an amount that provides a therapeutic benefit in the treatment, prevention, or management of a cancer, e.g. an amount that provides a statistically significant decrease in at least one symptom of a cancer. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, the term “pharmaceutical composition” refers to the active agent in combination with a pharmaceutically acceptable carrier of chemicals and compounds commonly used in the pharmaceutical industry.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a “subject” means a human or animal. In one embodiment, the animal is a vertebrate such as a primate, rodent, domestic animal, avian species, fish or game animal. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used as subjects that represent animal models of cancer, e.g., melanoma. In addition, the methods described herein can be used to treat domesticated animals and/or pets. A subject can be male or female. A subject can be one who has been previously diagnosed with cancer, e.g., melanoma, or a subject identified as having one or more complications related to cancer, and optionally, but need not have already undergone treatment for the cancer or the one or more complications related to the cancer. In one embodiment, the subject is selected for having cancer and can include, for example, a subject who has been identified or selected as having a resistant form of cancer, e.g., melanoma, e.g., a melanoma that is BRAF^{V600E} positive and does not respond to treatment with a BRAF^{V600E} specific small molecule drug. A subject can also be one who has been diagnosed with or identified as having one or more complications related to cancer.

Other than in the operating examples, or where otherwise indicated, all numbers expressing quantities of ingredients or reaction conditions used herein should be understood as modified in all instances by the term “about.” The term “about” when used in connection with percentages can mean±1%.

The term “statistically significant” or “significantly” refers to statistical significance and generally means a two standard deviation (2SD) difference, above or below a reference value.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### Wnt/β-Catenin Signaling

Activation of the Wnt/β-catenin signaling pathway normally occurs when binding of Wnt ligand to cognate FZD and LRP516 receptors leads to the stabilization and nuclear translocation of β-catenin, resulting in the regulation of target gene expression through its interaction with members of the TCF/LEF family of transcription factors. Clinically, the presence of nuclear β-catenin has been used as a surrogate indicator of Wnt/β-catenin activation, and the increased nuclear β-catenin observed in the majority of benign nevi implicates the presence of active Wnt/β-catenin signaling in these contexts. Decreased nuclear β-catenin is observed with melanoma progression, and the decreased survival seen in patients exhibiting lower levels of nuclear β-catenin in their tumors suggests that the loss of Wnt/β-catenin signaling plays an important role during melanoma evolution. In a transgenic mouse model, constitutive activation of Wnt/β-catenin signaling on its own does not result in spontaneous melanomas. This is in contrast to the effect observed in many other cancer types, where activated Wnt/β-catenin signaling can promote disease progression.

Activation of Wnt/β-catenin signaling promotes the nuclear functions of β-catenin (CTNNB1), resulting in the regulation of cell proliferation, differentiation, and behavior (8). The exact role of Wnt/β-catenin signaling in melanoma progression remains controversial. While transgenic mouse models using a melanocyte-specific, constitutively-active β-catenin mutant did not display any spontaneous melanomas, these mice exhibited enhanced immortalization of melanocytes and increased melanoma tumor promotion when combined with a concomitant activating mutation of Nras (9). By contrast, the decreased survival observed in patients exhibiting lower levels of nuclear β-catenin in their tumors suggests that the loss of Wnt/β-catenin signaling plays an important role during melanoma evolution (10-14). Although benign nevi and a significant number of melanoma tumors exhibit nuclear β-catenin (10,11,13,14), the presence of activating mutations in this context is rare (15,16), supporting a model in which the activation of Wnt/β-catenin signaling is mediated by Wnt ligand (17).

Components of the Wnt/β-catenin signaling pathway are known to those of ordinary skill in the art. For example, in humans, components of the Wnt/β-catenin pathway that can positively regulate Wnt/β-catenin signaling can include LRP5 (NCBI Gene ID No:4041); LRP6 (NCBI Gene ID No; 4040); FZD1 (NCBI Gene ID No; 8321); FZD2 (NCBI Gene ID No: 2535); FZD3 (NCBI Gene ID No: 7976); FZD4 (NCBI Gene ID No: 8322); FZD5 (NCBI Gene ID No: 7855); FZD6 (NCBI Gene ID No: 8323); FZD7 (NCBI Gene ID No: 8324); FZD8 (NCBI Gene ID No: 8325); FZD9 (NCBI Gene ID No:8326); FZD10 (NCBI Gene ID No: 11211); β-catenin (CTNNB1, NCBI Gene ID No:1499); TCF1 (NCBI Gene ID No: 6927); LEF1 (NCBI Gene ID No: 51176) TCF3 (NCBI Gene ID No: 6929); TCF4 (NCBI Gene ID No: 6929); PORCN (NCBI Gene ID No: 64840); WLS (NCBI Gene ID No: 79971); FLOT2 (NCBI Gene ID No: 2319); GPC4 (NCBI Gene ID No: 2239), GPC5 (NCBI Gene ID No: 2262); PPP1CA (NCBI Gene ID No: 5499); PPP1CB (NCBI Gene ID No: 5500); PPP1CC(NCBI Gene ID No: 5501); MACF1 (NCBI Gene ID No: 23499), CAPRIN2 (NCBI Gene ID No: 65981). Components of the Wnt/β-catenin pathway that can negatively regulate Wnt/β-catenin signaling can include AXIN1 (NCBI Gene ID No: 8312); AXIN2 (NCBI Gene ID No: 8313); RYK (NCBI Gene ID No: 6259); ROR2 (NCBI Gene ID No: 4920); SFRP1 (NCBI Gene ID No: 6422); SFRP2 (NCBI Gene ID No: 6423); FRZB (NCBI Gene ID No:2487); SFRP4 (NCBI Gene ID No: 6424), SFRP5 (NCBI Gene ID No: 6425); WIF1 (NCBI Gene ID No: 11197); DKK1 (NCBI Gene ID No: 22943); DKK2 (NCBI Gene ID No: 27123); DKK3 (NCBI Gene ID No: 27122); DKK4 (NCBI Gene ID No: 27121); SOST (NCBI Gene ID No: 50964); SOSTCD1 (NCBI Gene ID No: 25928); KREMEN1 (NCBI Gene ID No: 83999); KREMEN2 (NCBI Gene ID No: 79412); SHISA2 (NCBI Gene ID No: 387914); SHISA3 (NCBI Gene ID No: 152573); SHISA4 (NCBI Gene ID No: 149345); SHISA5 (NCBI Gene ID No: 51246); SHISA6 (NCBI Gene ID No:388336); SHISA7 (NCBI Gene ID No: 729956); SHISA8 (NCBI Gene ID No: 440829); SHISA9 (NCBI Gene ID No: 729993); CER1 (NCBI Gene ID No: 9350); IFGBP4 (NCBI Gene ID No:3487); NDP (NCBI Gene ID No: 4693); RSPO1 (NCBI Gene ID No: 284654); GSK3β (NCBI Gene ID No: 2932), CSNK1A1 (NCBI Gene ID No:1452); ANKRD6 (NCBI Gene ID No: 22881); and FAM123B (NCBI Gene ID No:139285). Components of the Wnt/β-catenin pathway that can either positively or negatively regulate Wnt/β-catenin signaling depending upon the context include PPP2CB (NCBI Gene ID No: 5516); PPP2R1A (NCBI Gene ID No: 5518); PPP2R1B (NCBI Gene ID No:5519); PPP2R2A (NCBI Gene ID No: 5520); PPP2R5D (NCBI Gene ID No: 5528); and APC (NCBI Gene ID No: 324).

The Wnt signalling pathway is described in Thorstensen et al., “WNT-inducible Signaling Pathway Protein 3, WISP-3, is Mutated in Microsatellite Unstable Gastrointestinal Carcinomas but Not in Endometrial Carcinomas,” Atlas Genet Cytogenet Oncol Haematol 7(2): 300-331 (2003), which is hereby incorporated by reference in its entirety. Detailed reviews of Wnt signalling and action are set out in Logan et al., “The Wnt Signaling Pathway in Development and Disease,” Annu Rev Cell Dev Biol 20:781-810 (2004); Wodarz et al., “Mechanisms of Wnt Signaling in Development,” Annu Rev Cell Dev Biol 14:59-88 (1998), which are hereby incorporated by reference in their entirety. The latter document also describes a number of assays for Wnt signalling.

Although Wnt/β-catenin signaling and ERK signaling have been suggested to engaged in cross-talk (21,22), the data has been somewhat contradictory and such a relationship has not been investigated in the context of melanoma. Further, as demonstrated elsewhere herein, the relationship between Wnt/β-catenin signaling and ERK signaling varies between melanocytes and melanoma cells.

### ERK Signaling

The extracellular signal-regulated kinases (ERKs) are activated by multiple signals including growth factors, cytokines, transforming growth factors, and G protein-coupled receptors (18). These signals lead to activation of RAS small G proteins which activate RAF kinases. Active RAF kinases phosphorylate and activate MEK kinases, which subsequently phosphorylate and activate ERK1/2 kinases. ERK1/2 kinases phosphorylate and regulate numerous substrates including other protein kinases, protein phosphatases, transcription factors, scaffolding proteins, signaling molecules and apoptosis-related proteins which lead to a variety of cell type and context-dependent responses (19). Constitutive activation of ERK1/2 by activating mutations in NRAS or BRAF is observed in the majority of melanomas and plays an integral role in the regulation of proliferation, invasiveness, and survival (20). In one embodiment, “ERK signaling” is signaling involving or mediated by the kinase activity of ERK1/2 kinases. In another embodiment, ERK signaling comprises signal transduction via downstream targets of ERK1/2 kinase activity.

Components of the ERK signaling pathway are known to those of ordinary skill in the art. For example, in humans, components of the ERK signaling pathway that can positively regulate ERK signaling include, for example, BRAF (NCBI Gene ID No: 673); EGFR (NCBI Gene ID No: 1956); HER2 (NCBI Gene ID No: 2064); c-KIT (NCBI Gene ID No: 3815); MET (NCBI Gene ID No: 4233); MEK1 (NCBI Gene ID No: 5604); MEK2 (NCBI Gene ID No: 5605); ERK1 (NCBI Gene ID No: 5595); ERK2 (NCBI Gene ID No: 5594); HRAS (NCBI Gene ID No: 3265); KRAS (NCBI Gene ID No: 3845); and NRAS (NCBI Gene ID No: 4893).

Components of the ERK signaling pathway that can negatively regulate ERK signaling include, for example, SGK1 (NCBI Gene ID No: 6446); IGFBP7 (NCBI Gene ID No: 3490); SPRED1 (NCBI Gene ID No: 161742); and KSR1 (NCBI Gene ID No: 8844).

### Inhibitors of ERK Signaling

Described herein are methods involving the inhibition of ERK signaling, e.g., for treatment of melanoma in subjects in need thereof. As used herein, the term “inhibitor of ERK signaling” refers to a compound or agent, such as a small molecule, that inhibits, decreases, lowers, or reduces the level of ERK signaling. An inhibitor of ERK signaling can be an antagonist of any component of the ERK signaling pathway that positively regulates ERK signaling, e.g. BRAF or MEK, or an agent which decreases the amount or activity of those components, e.g. an RNAi molecule. An inhibitor of ERK signaling can be an agonist of any component of the ERK signaling pathway which negatively regulates ERK signaling, or an agent which increases the amount or activity of those components. In some embodiments, an inhibitor of ERK signaling specifically inhibits the kinase activity of one or more RAF kinases or an ortholog thereof, e.g., it decreases the phosphorylation of one or more MEK kinases. In some embodiments, an inhibitor of ERK signaling is a specific inhibitor of the activity of BRAF. In some embodiments, an inhibitor of ERK signaling is a specific inhibitor of the activity of a mutant form of BRAF. In some embodiments, an inhibitor of ERK signaling is a specific inhibitor of the activity of BRAF^{V600E}. In some embodiments, an inhibitor of ERK signaling specifically inhibits the kinase activity of one or more MEK kinase or an ortholog thereof, e.g., it decreases the phosphorylation of ERK1/2. In some embodiments, an inhibitor of ERK signaling specifically inhibits the kinase activity of one or more of ERK1 and ERK2 kinases or an ortholog thereof, e.g., it decreases the phosphorylation of a substrate of ERK1/2.

The terms “decrease,” “reduce,” “reduced”, “reduction”, “decrease,” “suppress,” “inhibit,” or “inhibition” are all used herein generally to mean a decrease by a statistically significant amount. However, for avoidance of doubt, “reduce,” “reduction” or “decrease” or “inhibit” in regard to inhibition of ERK signaling by an inhibitor of ERK signaling, as described herein, typically means a decrease by at least about 10% as compared to the absence of the treatment, for example a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% decrease or more, i.e. absent level, as compared to the absence of the treatment, or any decrease between 10-99% as compared to in the absence of the treatment.

Inhibition of ERK signaling can be measured according to methods well-known to those of ordinary skill in the art. By way of non-limiting example, inhibition of ERK signaling can be measured by determining the level of dual-phosphorylated ERK1/2 (ppERK1/2) as described in detail elsewhere herein. In brief, the level of ppERK1/2 can be detected by immunoblot assay. Contacting a cell with an agent that is an inhibitor of ERK signaling will cause the cell to exhibit a lower level of ppERK1/2 than a cell not contacted with the agent.

As used herein, the term “inhibitor of BRAF” refers to a compound or agent, such as a small molecule, that inhibits, decreases, lowers, or reduces the activity of any of the isoforms or mutants of BRAF, e.g. kinase activity that phosphorylates MEK. As used herein, the term “inhibitor of a BRAF mutant” refers to a compound or agent, such as a small molecule, that inhibits, decreases, lowers, or reduces the activity of one or more mutant forms of BRAF. As used herein, the term “inhibitor of BRAF^{V600E}” refers to a compound or agent, such as a small molecule, that inhibits, decreases, lowers, or reduces the activity of BRAF^{V600E}. An inhibitor of BRAF or an inhibitor of a BRAF mutant or an inhibitor of BRAF^{V600E} can selectively inhibit at least one isoform or mutant of BRAF. In some embodiments, a selective inhibitor can be an inhibitor that inhibits the activity only of the desired target. In some embodiments, a selective inhibitor can be an inhibitor that inhibits the activity of the desired target at least 20-fold or more, e.g. 30-fold or more, 50-fold or more, 100-fold or more, 200-fold or more, or 500-fold or more than the degree to which it inhibits any other protein present in the subject to which it is administered. In some embodiments, a selective inhibitor can be an agent with an IC₅₀ less than 1 μM, e.g., less than 500 nM, less than 10 nM, 80 nM, less than 70 nM, less than 50 nM or lower.

Examples of inhibitors of BRAF include, but are not limited to, PLX4720 (N-[3-[(5-Chloro-1H-pyrrolo[2,3-b]pyridin-3-yl)carbonyl]-2,4-difluorophenyl]-1-propanesulfonamide; Structure I), PLX4032 (vemurafenib; RG7204; N-[2,4-Difluoro-3-[[5-(3-pyridinyl)-1H-pyrrolo[2,3-b]pyridin-3-yl]carbonyl]phenyl]-2-propanesulfonamide; Structure II) and GSK2118436 (5-[2-[4-[2-(Dimethylamino)ethoxy]phenyl]-5-(4-pyridinyl)-1H-imidazol-4-yl]-2,3-dihydro-1H-inden-1-one oxime; Structure III). Further non-limiting examples of BRAF inhibitors include dasatinib, erlotinib, geftinib, imatinib, lapatinib, sorafenib, sunitinib, dexanabinol, PD-325901, XL518, PD-318088, RG7204, GDC-0879, and sorafenib losylate (Bay 43-9006) or a derivative or pharmaceutically acceptable salt thereof. These and other inhibitors of BRAF, as well as non-limiting examples of their methods of manufacture, are described in US Patent Publications US2005/0176740, US2011/0020217, US2007/0078121, US2011/0118298, U.S. Pat. No. 4,876,276; International Patent Applications WO02/24680, WO03/022840, WO07/002,325 the contents of which are herein incorporated by reference in their entireties.

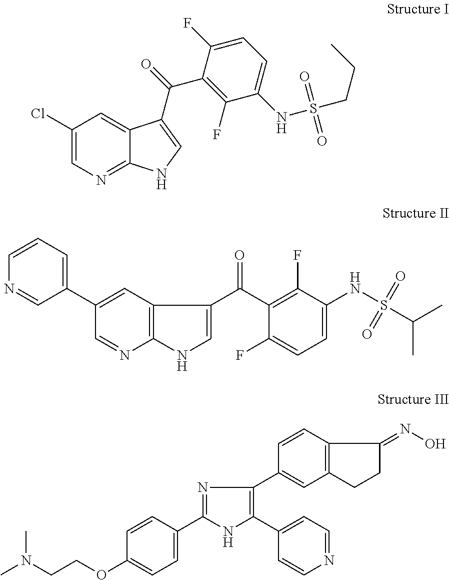

Commerically available BRAF inhibitors include, but are not limited to, compounds such as PLX4720 (Cat# SY-PLX4720; Symansis, Australia), or sorafenib, which is marketed as Nexavar by Bayer/Onyx.

In some embodiments, the inhibitor of ERK signaling can be an inhibitor of MEK. As used herein, the term “inhibitor of MEK” refers to a compound or agent, such as a small molecule, that inhibits, decreases, lowers, or reduces the activity of MEK.

Examples of inhibitors of MEK include, but are not limited to, AZD6244 (6-(4-Bromo-2-chloro-phenylamino)-7-fluoro-3-methyl-3H-benzoimidazole-5-carboxylic acid (2-hydroxy-ethoxy)-amide; selumetinib; Structure IV), and U0126 (1,4-diamino-2,3-dicyano-1,4-bis [2-aminophenylthio]butadiene; ARRY-142886; Structure V). Further non-limiting examples of MEK inhibitors include PD0325901, AZD2171, GDC-0973/XL-518, PD98059, PD184352, GSK1120212, RDEA436, RDEA119/BAY869766, AS703026, BIX 02188, BIX 02189, CI-1040 (PD184352), PD0325901, and PD98059. These and other inhibitors of MEK, as well as non-limiting examples of their methods of manufacture, are described in U.S. Pat. Nos. 5,525,625; 6,251,943; 7,820,664; 6,809,106; 7,759,518; 7,485,643; 7,576,072; 7,923,456; 7,732,616; 7,271,178; 7,429,667; 6,649,640; 6,495,582; 7,001,905; US Patent Publication No. US2010/0331334, US2009/0143389, US2008/0280957, US2007/0049591, US2011/0118298, International Patent Application Publication No. WO98/43960, WO99/01421, WO99/01426, WO00/41505, WO00/42002, WO00/42003, WO00/41994, WO00/42022, WO00/42029, WO00/68201, WO01/68619, WO02/06213 and WO03/077914, the contents of which are herein incorporated by reference in their entireties.

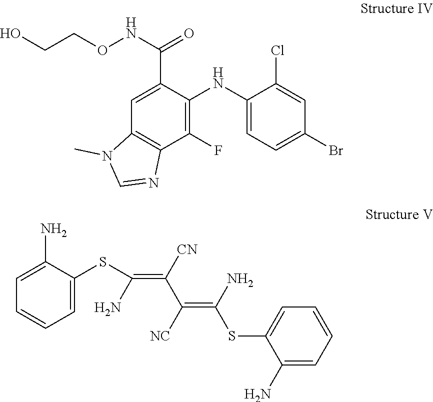

Commercially available MEK inhibitors include, but are not limited to, U0126 (Cat#9903; Cell Signaling Technology, Danvers, Mass.) and AZD6244 (selumetinib) which is being developed by AstraZeneca (Cat No # S1008; Selleck, Houston, Tex.).

### Activators of Wnt/β-Catenin Signaling

Embodiments of the methods described herein employ activators of Wnt/β-catenin signaling. As used herein, the term “activator of Wnt/β-catenin signaling” refers to a compound or agent, including, but not limited to, a small molecule, that increases the level of Wnt/β-catenin signaling. In some embodiments, an activator of Wnt/β-catenin signaling can bind to and increase the activity of a Wnt/β-catenin pathway receptor, e.g., a Frizzled receptor. In some embodiments, an activator of Wnt/β-catenin signaling can be an inhibitor of GSK3β. At a minimum, an activator of the Wnt/β-catenin pathway will result in the accumulation of nuclear β-catenin and β-catenin trans-activation of, for example, AXIN2 expression. Alternatively, or in addition, as noted below, activation of the BAR reporter gene can be used as an indicator of Wnt/β-catenin signaling in cultured cells.

An activator of Wnt/β-catenin signaling is to be distinguished from an enhancer of Wnt/β-catenin signaling. An enhancer can increase the effect of an activator but unlike and “activator of Wnt/β-catenin signaling”, is not, in and of itself sufficient to increase the level of Wnt/β-catenin signaling. Without wishing to be bound by theory, an enhancer can work by, for example, modulating a pathway which is linked to Wnt/β-catenin signaling by cross-talk. An enhancer can be efficacious when administered to a subject or a cell prior to, concurrently with, and/or following administration of an activator. Thus, an activator of Wnt/β-catenin signaling can, on its own, induce activity of the Wnt/β-catenin signaling pathway

The terms “increased”, “increase” or “activate” are all used herein to generally mean an increase by a statistically significant amount relative to a reference; for the avoidance of any doubt, the terms “increased”, “increase” or “activate” means an increase of at least about 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

Activation of Wnt/β-catenin signaling can be measured by methods well-known to those of ordinary skill in the art. By way of non-limiting example, Wnt/β-catenin signaling can be measured using the BAR reporter described in detail elsewhere herein. Briefly, the BAR (β-catenin activated reporter) is a lentiviral plasmid which provides for expression of luciferase in response to Wnt/β-catenin signaling. Output can be measured with an automated luminescence plate reader. Higher luminescence in the presence of an agent, as compared to in the absence of the agent indicates that the agent is an activator of Wnt/β-catenin signaling.

The expression level of a gene that is a marker for activation of the Wnt/β-catenin pathway can also be used to measure activation of Wnt/β-catenin signaling. The expression level of the marker gene for activation of the Wnt/β-catenin pathway can be determined by a variety of techniques, including immunoassays (e.g., enzyme linked immunoabsorbant assay (ELISA), radioimmunoassay (RIA), immunoradiometric assay (IRMA)), Western blotting, PCR, or immunohistochemistry (including AQUA®). Of these, quantitative PCR is particularly useful.

Genes expressed as a result of activation of the Wnt/β-catenin pathway are numerous and well known to those of ordinary skill in the art. Such genes can include, for example, LRP5 (NCBI Gene ID No:4041); LRP6 (NCBI Gene ID No; 4040); FZD1 (NCBI Gene ID No; 8321); FZD2 (NCBI Gene ID No: 2535); FZD3 (NCBI Gene ID No: 7976); FZD4 (NCBI Gene ID No: 8322); FZD5 (NCBI Gene ID No: 7855); FZD6 (NCBI Gene ID No: 8323); FZD7 (NCBI Gene ID No: 8324); FZD8 (NCBI Gene ID No: 8325); FZD9 (NCBI Gene ID No:8326); FZD10 (NCBI Gene ID No: 11211); β-catenin (CTNNB1, NCBI Gene ID No:1499); TCF1 (NCBI Gene ID No: 6927); LEF1 (NCBI Gene ID No: 51176) TCF3 (NCBI Gene ID No: 6929); TCF4 (NCBI Gene ID No: 6929); AXIN1 (NCBI Gene ID No: 8312); AXIN2 (NCBI Gene ID No: 8313); DKK1 (NCBI Gene ID No: 22943); DKK2 (NCBI Gene ID No: 27123); DKK3 (NCBI Gene ID No: 27122); DKK4 (NCBI Gene ID No: 27121); KREMEN1 (NCBI Gene ID No: 83999); KREMEN2 (NCBI Gene ID No: 79412); GSK3β (NCBI Gene ID No: 2932), and APC (NCBI Gene ID No: 324).

In certain embodiments, the activator of Wnt/β-catenin is a small molecule. By way of a non-limiting example, SLK2001 (Gwak et al., Cell Res 2011, published online on Aug. 9, 2011 ahead of print) is an activator of Wnt/β-catenin signaling.

In some embodiments, activators of Wnt/β-catenin can be agonists of a component of the Wnt/β-catenin signaling pathway. In some embodiments, an agonist of a component of the Wnt/β-catenin signaling pathway can be a Wnt ligand.

A “Wnt ligand” is any member of a family of highly conserved secreted signaling molecules that will bind Wnt cell surface receptors of the Frizzled family. A list of Wnt ligands for various species is available on the world wide web at stanford.edu/rnusse/wntwindow.html. For example, Wnt ligands (and the GenBank accession number for their transcript) in the mouse include Wnt1 (int-1, NM_{—}021279), Wnt2 (irp, NM_{—}023653), Wnt2b/13 (NM_{—}009520), Wnt3 (NM_{—}009521), Wnt3a (NM_{—}009522), Wnt4 (NM_{—}009523), Wnt5a (NM_{—}009524), Wnt5b (NM_{—}009525), Wnt6 (NM_{—}009526), Wnt7a (NM_{—}009527), Wnt7b (NM_{—}009528), Wnt8a (NM_{—}009290), Wnt8b (NM_{—}011720), Wnt9a (Wnt14, NM 139298), Wnt9b (Wnt15, NM_{—}011719), Wnt10a (NM_{—}009518), Wnt10b (NM_{—}011718), Wnt11 (NM_{—}009519), and Wnt16 (NM_{—}053116). Wnt ligands (and the GenBank accession number for their transcript) in humans include Wnt1 (NM_{—}005430), Wnt2 (NM_{—}003391), Wnt2b/13 (NM_{—}024494 and NM_{—}004185), Wnt3 (NM_{—}030753), Wnt3a (NM_{—}033131), Wnt4 (NM_{—}030761), Wnt5a (NM_{—}003392), Wnt5b (NM_{—}032642), Wnt6 (NM_{—}006522), Wnt7a (NM_{—}004625), Wnt7b (NM_{—}058238), Wnt8a (NM_{—}058244), Wnt8b (NM_{—}003393), Wnt9a (Wnt14, NM_{—}003395), Wnt9b (Wnt15, NM_{—}003396), Wnt10a (NM_{—}025216), Wnt10b (NM_{—}003394), Wnt11 (NM_{—}004626) and Wnt16 (NM_{—}057168). These ligands, as well as non-limiting examples of their methods of manufacture, are described in the contents of US Patent Publications US2010/0199362 and US2008/0193515, which are herein incorporated by reference in their entireties.

The activator can be in the form of a nucleic acid comprising a nucleotide sequence that encodes a Wnt polypeptide; a polypeptide comprising an amino acid sequence of a Wnt polypeptide, a nucleic acid comprising a nucleotide sequence that encodes an activated Wnt receptor, a polypeptide comprising an amino acid sequence of an activated Wnt receptor, a small organic molecule that promotes Wnt/β-catenin signaling, a small organic molecule that inhibits the expression or activity of a Wnt or β-catenin antagonist, an antisense oligonucleotide that inhibits expression of a Wnt or β-catenin antagonist, a ribozyme that inhibits expression of a Wnt or β-catenin antagonist, an RNAi construct, siRNA, or shRNA that inhibits expression of a Wnt or β-catenin antagonist, an antibody that binds to and inhibits the activity of a Wnt or β-catenin antagonist, e.g., GSK3β, a nucleic acid comprising a nucleotide sequence that encodes a Lef-1 polypeptide, and/or a polypeptide comprising an amino acid sequence of Lef-1 polypeptide.

In some embodiments, activators of Wnt/β-catenin signaling can be inhibitors of an antagonist of Wnt/β-catenin. By way of non-limiting example, an activator of Wnt/β-catenin signaling can be a GSK3β inhibitor.

Examples of GSK3β inhibitors include, but are not limited to, CHIR-99021 (CHIR-911; CT-99021; 6-[[2-[[4-(2,4-dichlorophenyl)-5-(5-methyl-1H-imidazol-2-yl)-2-pyrimidinyl]amino]ethyl]amino]-3-pyridinecarbonitrile; Structure VI). Further examples of GSK3β inhibitors include CHIR-837 (CT-98023; Chiron Corporation (Emeryville, Calif.)), SB236763, riluzole, flunarizine, 6-bromoindirubin-3′-oxime (BIO), CHIR-98014, CHIR-99030, and CHIR-98023. These and other GSK3β inhibitors, as well as non-limiting examples of their methods of manufacture, are described in U.S. Pat. Nos. 6,057,117 and 6,608,063; U.S. Patent Publication Nos. 2004/0092535, 2004/0209878 and International Patent Publication WO01/056662, the contents of which are herein incorporated by reference in their entireties.

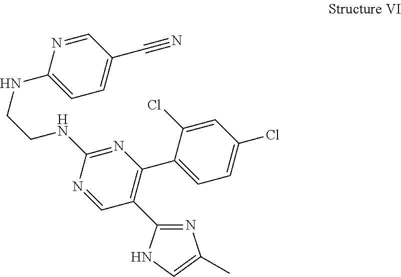

In some embodiments, the inhibitor of an antagonist of Win/β-catenin can be an agent that decreases or lowers the expression or activity of AXIN1.

### Agents

The terms “compound” and “agent” refer to any entity which is normally not present or not present at the levels being administered to a cell, tissue or subject. Agent can be selected from a group comprising: chemicals; small organic or inorganic molecules; nucleic acid sequences; nucleic acid analogues; proteins; peptides; aptamers; peptidomimetic, peptide derivative, peptide analogs, antibodies; intrabodies; biological macromolecules, extracts made from biological materials such as bacteria, plants, fungi, or animal cells or tissues; naturally occurring or synthetic compositions or functional fragments thereof. In some embodiments, the agent is any chemical, entity or moiety, including without limitation synthetic and naturally-occurring non-proteinaceous entities. In certain embodiments the agent is a small molecule having a chemical moiety. For example, chemical moieties includes unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

In certain embodiments, an agent can increase or decrease the expression of a component of the targetted signaling pathway. Transcriptional assays are well known to those of skill in the art (see e.g. U.S. Pat. Nos. 7,319,933, 6,913,880,).

Gene silencing or RNAi can be used. In certain embodiments, contacting a cell with the agent results in a decrease in the mRNA level in a cell for a target gene by at least about 10%, e.g., at least about 20%, about 30%, about 40%, about 50%, about 60%, about 70%, about 80%, about 90%, about 95%, about 99%, or more of the mRNA level found in the cell without the presence of the miRNA or RNA interference molecule. In one embodiment, the mRNA levels are decreased by at least about 70%, about 80%, about 90%, about 95%, about 99%, or more, i.e., no detectable target mRNA. In certain embodiments, the agent comprises an expression vector or viral vector comprising the RNAi molecule. Methods of assaying the ability of an agent to inhibit translation of a gene are known to those of ordinary skill in the art. Gene translation can be measured by quantitation of protein expressed from a gene, for example by Western blotting, by an immunological detection of the protein, ELISA (enzyme-linked immunosorbent assay), Western blotting, radioimmunoassay (RIA) or other immunoassays and fluorescence-activated cell analysis (FACS) to detect protein.

In some embodiments, in order to increase nuclease resistance in an agent comprising a nucleic acid as disclosed herein, one can incorporate non-phosphodiester backbone linkages, as for example methylphosphonate, phosphorothioate or phosphorodithioate linkages or mixtures thereof. Other functional groups may also be joined to the oligonucleoside sequence to instill a variety of desirable properties, such as to enhance uptake of the oligonucleoside sequence through cellular membranes, to enhance stability or to enhance the formation of hybrids with the target nucleic acid, or to promote cross-linking with the target (as with a psoralen photo-cross-linking substituent). See, for example, PCT Publication No. WO 92/02532 which is incorporated herein in by reference.

The agent may comprise a vector. Many vectors useful for transferring exogenous genes into target mammalian cells are available, e.g. the vectors may be episomal, e.g., plasmids, virus derived vectors such cytomegalovirus, adenovirus, etc., or may be integrated into the target cell genome, through homologous recombination or random integration, e.g., retrovirus derived vectors such MMLV, HIV-1, ALV, etc. Many viral vectors are known in the art and can be used as carriers of a nucleic acid modulatory compound into the cell. For example, constructs containing the modulatory compound may be integrated and packaged into non-replicating, defective viral genomes like Adenovirus, Adeno-associated virus (AAV), or Herpes simplex virus (HSV) or others, including retroviral and lentiviral vectors, for infection or transduction into cells. Alternatively, the construct may be incorporated into vectors capable of episomal replication, e.g. EPV and EBV vectors. The nucleic acid incorporated into the vector can be operatively linked to an expression control sequence when the expression control sequence controls and regulates the transcription and translation of that polynucleotide sequence.

In certain embodiments, the agent is a protein or peptide. A peptide agent can be a fragment of a naturally occurring protein, or a mimic or peptidomimetic. Agents in the form of a protein and/or peptide or fragment thereof can be designed to increase or decrease the level of a gene or protein involved in Wnt/β-catenin signaling or ERK signaling as described herein, i.e. increase or decrease gene expression or encoded protein activity. Such agents are intended to encompass proteins which are normally absent as well as proteins normally endogenously expressed within a cell, e.g. expressed at low levels. Examples of useful proteins are mutated proteins, genetically engineered proteins, peptides, synthetic peptides, recombinant proteins, chimeric proteins, modified proteins and fragments thereof. An increase or decrease in gene expression or protein activity can be direct or indirect. In one embodiment, a protein/peptide agent directly binds to a protein which is a component of the targeted signaling pathway, or directly binds to a nucleic acid which encodes such a protein.

In one embodiment, protein/peptide agents (including antibodies, or fragments thereof) can be assessed for their ability to bind an encoded protein in vitro. Examples of direct binding assays include, but are not limited to, labeled in vitro protein-protein binding assays, electrophoretic mobility shift assays, immunoassays for protein binding, ELISA assays, co-immunoprecipitation assays, competition assays (e.g. with a known binder), and the like. See, e.g., U.S. Pat. Nos. 4,366,241; 4,376,110; 4,517,288; and 4,837,168; and also Bevan et al., Trends in Biotechnology 13:115-122, 1995; Ecker et al., Bio/Technology 13:351-360, 1995; and Hodgson, Bio/Technology 10:973-980, 1992. The agent can also be assayed or identified by detecting a signal that indicates that the agent binds to a protein of interest e.g., fluorescence quenching or FRET. Polypeptides can also be monitored for their ability to bind nucleic acid in vitro, e.g. ELISA-format assays can be a convenient alternative to gel mobility shift assays (EMSA) for analysis of protein binding to nucleic acid. Binding of an agent to an encoded protein provides an indication the agent may increase or decrease protein activity.

In certain embodiments, the agent is an antibody (See, generally, Hood et al., Immunology, Benjamin, N.Y., 2ND ed. (1984), Harlow and Lane, Antibodies. A Laboratory Manual, Cold Spring Harbor Laboratory (1988) and Hunkapiller and Hood, Nature, 323, 15-16 (1986), which are incorporated herein by reference). Monoclonal antibodies are prepared using methods well known to those of skill in the art. Methods for intrabody production are well known to those of skill in the art, e.g. as described in WO 2002/086096. Antibodies will usually bind with at least a KD of about 30 μM, preferably at least about 10 μM, and more preferably at least about 3 μM or better, e.g., 100 μM, 50 μM, 1 μM or better.

An agent can be a naturally occurring protein or a fragment thereof. Such agents can be obtained from a natural source, e.g., a cell or tissue lysate. The agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the agents are polypeptides or proteins.

An agent can function directly in the form in which it is administered. Alternatively, the agent can be modified or utilized intracellularly to produce something which is an inhibitor of ERK signaling or an activator of Wnt/β-catenin as described herein, e.g. introduction of a nucleic acid sequence into the cell and its transcription resulting in the production of an inhibitor or activator of gene expression or protein activity.

Agents can be produced recombinantly using methods well known to those of skill in the art (see Sambrook et al., Molecular Cloning: A Laboratory Manual (3 ed.), Cold Spring Harbor Laboratory Press, Cold Spring Harbor, N.Y., USA (2001)).

### Inhibitors of AXIN1

Described herein are methods involving the inhibition of AXIN1, e.g., for treatment of melanoma in subjects in need thereof. As used herein, the term “inhibitor of AXIN1” refers to a compound or agent, such as a small molecule or an RNAi molecule which inhibits, decreases, lowers, or reduces the level and/or activity of AXIN1. In some embodiments, an inhibitor of AXIN1 reduces the level of the AXIN1 gene products, e.g., AXIN1 mRNA (SEQ ID NOs:01-02) or AXIN1 protein (SEQ ID NOs: 03-04). In some embodiments, an inhibitor of AXIN1 reduces the activity of AXIN1 and/or reduces the interaction of AXIN1 with other proteins of the Wnt/β-catenin signaling pathway

Inhibition of AXIN1 can be measured according to methods well-known to those of ordinary skill in the art. By way of non-limiting example, inhibition of AXIN1 can be measured by determining the level of AXIN1 mRNA as described in detail elsewhere herein.

In some embodiments, an inhibitor of AXIN1 is an RNAi molecule specific for the AXIN1 mRNA. RNAi is described in detailed elsewhere herein.

In some embodiments, an inhibitor of AXIN1 is an antibody or antigen-binding fragment thereof which is specific for the AXIN1 protein. Such agents are described in detail elsewhere herein.

### Dosage and Administration

One aspect of the invention relates to a method of administering a therapeutically effective amount of an inhibitor of ERK signaling and a therapeutically effective amount of an activator of Wnt/β-catenin to a subject in need of treatment for melanoma. In some embodiments, the inhibitor of ERK signaling and the activator of Wnt/β-catenin can be administered as separate compositions. In some embodiments, a composition can comprise both an inhibitor of ERK signaling and an activator of Wnt/β-catenin signaling.

Suitable routes for administration of a composition of the present invention include but are not limited to peritoneal, subcutaneous, topical, or oral administration. In one embodiment of the methods described herein, the composition is administered orally. In one embodiment of the methods described herein, the composition is administered intravenously. The agents described herein can be administered in any manner found appropriate by a clinician, such as described on a product label, or in the clinical literature, or in the Physicians' Desk Reference, 56th Ed. (2002) Publisher Edward R. Barnhart, New Jersey (“PDR”).

As used herein, the term “administer” refers to the placement of a composition into a subject by a method or route which results in at least partial localization of the composition at a desired site such that a desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intra-cerebrospinal, and intrasternal injection and infusion. In preferred embodiments, the compositions are administered by intravenous infusion or injection.

The phrases “parenteral administration” and “administered parenterally” as used herein, refer to modes of administration other than enteral and topical administration, usually by injection, and includes, without limitation, intravenous, intraperitoneal, intramuscular, intraarterial, intrathecal, intraventricular, intracapsular, intraorbital, intracardiac, intradermal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein refer to the administration of the an agent as described herein other than directly into a target site, tissue, or organ, such as a surgical site, such that it enters the subject's circulatory system and, thus, is subject to metabolism and other like processes.

In one embodiment, the administration is systemic.

In one embodiment, the administration is locally directed to the tumor.

Dosage

In one embodiment, a therapeutically effective amount of a composition is administered to a subject. A “therapeutically effective amount” is an amount of a composition comprising an inhibitor of ERK signaling and/or an activator of Wnt/β-catenin signaling sufficient to produce a measurable improvement in a symptom or marker of melanoma. Actual dosage levels of active ingredients in a therapeutic composition can be varied so as to administer an amount of the active compound(s) that is effective to achieve the desired therapeutic response for a particular subject. The selected dosage level will depend upon a variety of factors including, but not limited to, the activity of the therapeutic composition, formulation, the route of administration, combination with other drugs or treatments, severity of disease and the physical condition, and prior medical history of the subject being treated and the experience and judgment of the clinician or practitioner administering the therapy. Generally, the dose and administration scheduled should be sufficient to result in slowing, and preferably inhibiting tumor growth and also preferably causing regression of the melanoma. In some cases, regression can be monitored by a decrease in blood levels of tumor specific markers. Determination and adjustment of a therapeutically effective dose, as well as evaluation of when and how to make such adjustments, are known to those of ordinary skill in the art of medicine.

In one embodiment of the methods described herein, a minimally therapeutic dose is administered. The term “minimally therapeutic dose” refers to the smallest dose, or smallest range of doses, determined to be a therapeutically effective amount as that term is used herein.

The dosage of an inhibitor of ERK signaling and an activator of Wnt/β-catenin signaling administered according to the methods described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to the treatment regimen.

The dosage should not be so large as to cause substantial adverse side effects. The dosage can also be adjusted by the individual physician in the event of any complication or based upon the subject's sensitivity to the agent. Typically, however, the dosage can range from 0.0001 mg/kg body weight to 500 mg/kg body weight. In some embodiments, the dose range can be from 0.01 mg/kg body weight to 100 mg/kg body weight. In some embodiments, the dose range can be from 0.1 mg/kg body weight to 50 mg/kg body weight. Effective doses may be extrapolated from dose-response curves derived from, for example, animal model test bioassays or systems.

A composition or compositions comprising an inhibitor of ERK signaling and/or an activator of Wnt/β-catenin signaling can be administered over a period of time, such as over a 5 minute, 10 minute, 15 minute, 20 minute, or 25 minute period. When multiple doses are administered, the doses can be separated from one another by, for example, one hour, three hours, six hours, eight hours, one day, two days, one week, two weeks, or one month.

After an initial treatment regimen, the treatments can be administered on a less frequent basis. For example, after administration biweekly for three months, administration can be repeated once per month, for six months or a year or longer. In some embodiments, administration is chronic, e.g., one or more doses daily over a period of weeks or months as necessary.

Administration of a composition comprising an inhibitor of ERK signaling and/or an activator of Wnt/β-catenin signaling can reduce levels of a marker or symptom of melanoma by at least about 10%, at least about 15%, at least about 20%, at least about 25%, at least about 30%, at least about 40%, at least about 50%, at least about 60%, at least about 70%, at least about 80% or at least about 90% or more.

Therapeutic compositions comprising an inhibitor of ERK signaling and/or an activator of Wnt/β-catenin signaling or functional derivatives thereof are optionally tested in one or more appropriate in vitro and/or in vivo animal models of disease, such as the murine model of melanoma described herein, to confirm efficacy, evaluate tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the LD₅₀ of the relevant formulation, and/or observation of any side-effects of an inhibitor of ERK signaling and/or an activator of Wnt/β-catenin signaling or functional derivatives thereof at various concentrations, e.g., as applied to the mass and overall health of the patient. In determining the effective amount of an inhibitor of ERK signaling and/or an activator of Wnt/β-catenin signaling and functional derivatives thereof to be administered in the treatment of melanoma, the physician evaluates, among other criteria, circulating plasma levels, formulation toxicities, and progression of the condition.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio LD₅₀/ED₅₀. Compositions that exhibit large therapeutic indices are preferred. A dose can be formulated in animal models to achieve a circulating plasma concentration range that includes the IC₅₀ (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

The dosage of such compounds lies preferably within a range of circulating concentrations that include the ED₅₀ with little or no toxicity. The dosage may vary within this range depending upon the dosage form employed and the route of administration utilized.

With respect to the therapeutic methods described herein, it is not intended that the administration of an inhibitor of ERK signaling and/or an activator of Wnt/β-catenin signaling be limited to a particular mode of administration, dosage, or frequency of dosing. All modes of administration are contemplated, including intramuscular, intravenous, inhalation, intranasal, oral, intraperitoneal, intravesicular, intraarticular, intralesional, subcutaneous, or any other route sufficient to provide a dose adequate to treat melanoma.

Pharmaceutical Formulations

In some embodiments, a pharmaceutical composition comprises an inhibitor of ERK signaling and/or an activator of Wnt/β-catenin signaling, and optionally a pharmaceutically acceptable carrier. The compositions can further comprise at least one pharmaceutically acceptable excipient.

The pharmaceutical composition can include suitable excipients, or stabilizers, and can be in solid or liquid form such as, tablets, capsules, powders, solutions, suspensions, or emulsions. Typically, the composition will contain from about 0.01 to 99 percent, preferably from about 5 to 95 percent of active compound(s), together with the carrier. The compounds, when combined with pharmaceutically or physiologically acceptable carriers, excipients, or stabilizers, whether in solid or liquid form such as, tablets, capsules, powders, solutions, suspensions, or emulsions, can be administered orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes, for example, that of the nose, throat, and bronchial tubes (e.g., by inhalation). For most therapeutic purposes, the compounds can be administered orally as a solid or as a solution or suspension in liquid form, via injection as a solution or suspension in liquid form, or via inhalation of a nebulized solution or suspension.

Some examples of materials that can be comprised by pharmaceutically-acceptable carriers include: sugars, such as lactose, glucose and sucrose; starches, such as corn starch and potato starch; cellulose, and its derivatives, such as sodium carboxymethyl cellulose, methylcellulose, ethyl cellulose, microcrystalline cellulose and cellulose acetate; powdered tragacanth; malt; gelatin; lubricating agents, such as magnesium stearate, sodium lauryl sulfate and talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol (PEG); esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; bulking agents, such as polypeptides and amino acids; serum component, such as serum albumin, HDL and LDL; C₂-C₁₂ alcohols, such as ethanol; and other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of an inhibitor of ERK signaling and/or an activator of Wnt/β-catenin signaling.

The term “pharmaceutically acceptable carrier” refers to a carrier for administration of a therapeutic agent. Such carriers include, but are not limited to, saline, buffered saline, dextrose, water, glycerol, ethanol, and combinations thereof. Each carrier must be “acceptable” in the sense of being compatible with the other ingredients of the formulation, for example the carrier does not decrease the impact of the agent on the treatment. In other words, a carrier is pharmaceutically inert. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents can include starch and gelatin, while the lubricating agent, if present, will generally be magnesium stearate, stearic acid or talc. If desired, the tablets can be coated with a material such as glyceryl monostearate or glyceryl distearate, to delay absorption in the gastrointestinal tract. Agents included in drug formulations are described further herein below.

Suitable formulations also include aqueous and non-aqueous sterile injection solutions which can contain anti-oxidants, buffers, bacteriostats, bactericidal antibiotics and solutes which render the formulation isotonic with the bodily fluids of the intended recipient. Aqueous and non-aqueous sterile suspensions can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients mannitol or another sugar, for example in the range of in one embodiment 10 to 100 mg/ml, in another embodiment about 30 mg/ml; phosphate-buffered saline (PBS), and any other formulation agents conventional in the art.

Parenteral Dosage Forms

Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared.

Suitable vehicles that can be used to provide parenteral dosage forms of an inhibitor of ERK signaling and/or an activator of Wnt/β-catenin signaling as disclosed herein are well known to those skilled in the art. Such carriers include sterile liquids, such as water and oils, with or without the addition of a surfactant and other pharmaceutically and physiologically acceptable carrier, including adjuvants, excipients or stabilizers. Examples include, without limitation: sterile water; water for injection USP; saline solution; glucose solution; aqueous vehicles such as but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, olive oil, ethyl oleate, isopropyl myristate, and benzyl benzoate.

Sterile compositions for parenteral administration may preferably be aqueous or non-aqueous solutions, suspensions or emulsions. The sterile compositions can include sterile aqueous solutions which can also contain buffers, diluents and other suitable additives such as, but not limited to, penetration enhancers, carrier compounds and other pharmaceutically acceptable carriers or excipients. Aqueous suspensions can further contain substances which increase the viscosity of the suspension including, for example, sodium carboxymethylcellulose, sorbitol and/or dextran.

Formulations useful in the methods described herein can also include surfactants. Many organized surfactant structures have been studied and used for the formulation of drugs. Suitable surfactants include fatty acids and/or esters or salts thereof, bile acids and/or salts thereof. In certain embodiments of the invention the surfactant can be anionic, cationic, or nonionic. The use of surfactants in drug products, formulations and in emulsions has been reviewed (Rieger, in Pharmaceutical Dosage Forms, Marcel Dekker, Inc., New York, N.Y., 1988, p. 285).

Vesicles, such as liposomes, have attracted great interest because of their specificity and the duration of action they offer from the standpoint of drug delivery. Liposomes are unilamellar or multilamellar vesicles which have a membrane formed from a lipophilic material and an aqueous interior. The aqueous portion contains the composition to be delivered. Liposomes can be cationic (Wang et al., Biochem. Biophys. Res. Commun., 1987, 147, 980-985), anionic (Zhou et al., Journal of Controlled Release, 1992, 19, 269-274), or nonionic (Hu et al. S. T. P. Pharma. Sci., 1994, 4, 6, 466). Liposomes can comprise a number of different phospholipids, lipids, glycolipids, and/or polymers which can impart specific properties useful in certain applications and which have been described in the art (Allen et al., FEBS Letters, 1987, 223, 42; Wu et al., Cancer Research, 1993, 53, 3765; Papahadjopoulos et al. Ann. N.Y. Acad. Sci., 1987, 507, 64; Gabizon et al. PNAS, 1988, 85, 6949; Klibanov et al. FEBS Lett., 1990, 268, 235; Sunamoto et al. Bull. Chem. Soc. Jpn., 1980, 53, 2778; Illum et al. FEBS Lett., 1984, 167, 79; Blume et al. Biochimica et Biophysica Acta, 1990, 1029, 91; Hughes et al. Methods Mol. Biol. 2010; 605:445-59; U.S. Pat. Nos. 4,837,028; 5,543,152; 4,426,330; 4,534,899; 5,013,556; 5,356,633; 5,213,804; 5,225,212; 5,540,935; 5,556,948; 5,264,221; 5,665,710; European Patents EP 0 445 131 B1; EP 0 496 813 B1; and European Patent Publications WO 88/04924; WO 97/13499; WO 90/04384; WO 91/05545; WO 94/20073; WO 96/10391; WO 96/40062; WO 97/0478).

The pharmaceutical compositions can be prepared and formulated as emulsions or microemulsions. Emulsions are typically heterogeneous systems of one liquid dispersed in another in the form of droplets usually exceeding 0.1 μm in diameter and have been described in the art. Microemulsion can be defined as a system of water, oil and amphiphile which is a single optically isotropic and thermodynamically stable liquid solution and can comprise surfactants and cosurfactants. Both of these drug delivery means have been described in the art. See, e.g., Remington: The Science and Practice of Pharmacy, 21st Ed., Lippincott, Williams, and Wilkins, Philadelphia Pa. (2005); and Ansel's Pharmaceutical Dosage Forms and Drug Delivery Systems, 9^{th} Ed., Lippincott, Williams, and Wilkins, Philadelphia, Pa. (2011).

Compounds that alter or modify the solubility of a pharmaceutically acceptable salt of an inhibitor of ERK signaling and/or an activator of Wnt/β-catenin signaling as disclosed herein can also be incorporated into the parenteral dosage forms of the disclosure, including conventional and controlled-release parenteral dosage forms. Such formulations can comprise a controlled-dosage form of an inhibitor of ERK signaling and/or an activator of Wnt/β-catenin signaling, e.g. a biodegradable hydrogel comprising an inhibitor of ERK signaling and/or an activator of Wnt/β-catenin signaling.

Oral Administration

Oral administration is preferred where the agent used can be formulation for such. Formulations for oral administration may be presented with an absorption enhancer. Orally-acceptable absorption enhancers include surfactants such as sodium lauryl sulfate, palmitoyl carnitine, Laureth-9, phosphatidylcholine, cyclodextrin and derivatives thereof; bile salts such as sodium deoxycholate, sodium taurocholate, sodium glycochlate, and sodium fusidate; chelating agents including EDTA, citric acid and salicylates; and fatty acids (e.g., oleic acid, lauric acid, acylcarnitines, mono- and diglycerides). Other oral absorption enhancers include benzalkonium chloride, benzethonium chloride, CHAPS (3-(3-cholamidopropyl)-dimethylammonio-1-propanesulfonate), Big-CHAPS(N, N-bis(3-D-gluconamidopropyl)-cholamide), chlorobutanol, octoxynol-9, benzyl alcohol, phenols, cresols, and alkyl alcohols. An especially preferred oral absorption enhancer is sodium lauryl sulfate. Oral formulations and their preparation are described in detail in U.S. Pat. No. 6,887,906, US Publn. No. 20030027780, and U.S. Pat. No. 6,747,014, each of which is incorporated herein by reference.

The oral formulations of the agents described herein, i.e. an inhibitor of ERK signaling and/or an activator of Wnt/β-catenin signaling, further encompass, in some embodiments, anhydrous pharmaceutical compositions and dosage forms comprising the agents as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Anhydrous pharmaceutical compositions and dosage forms can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected. Anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

Controlled-Release Formulations

In some embodiments, an an inhibitor of ERK signaling and/or an activator of Wnt/β-catenin signaling can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Chemg-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, Duolite® A568 and Duolite® AP143 (Rohm&Haas, Spring House, Pa. USA).

Combination Therapies

In some embodiments the methods for the treatment of melanoma as described herein can also be used in combination with any other therapy known in the art for the treatment of melanoma, symptoms and/or complications arising from melanoma or conditions which are associated with melanoma. An inhibitor of ERK signaling and/or an activator of Wnt/β-catenin signaling can be administered as the primary therapeutic agent or can be co-administered with one or more additional therapeutic agents. The methods described herein can be used in combination with other treatment methods used for treatment of melanoma that are well known to one skilled in the art. By way of non-limiting example, such methods include surgical excision of the cancerous skin lesion to reduce the chance of recurrence and preserve healthy skin tissue; chemotherapy; radiation therapy and administration of bacilli Calmette-Guerin (BCG) vaccine, bleomycin, interferon, or IL-2. Examples of chemotherapeutics accepted for use in the treatment of melanoma include, but are not limited to, dacarbazine (DTIC); temozolomide (Temodar); paclitaxel (Taxol); cisplatin (Paraplatin); carmustine (BCNU); fotemustine; vincristine (Oncovin, Vincasar) and vindesine (Eldisine, Fildesin).

Exemplary pharmaceutically active compounds include, but are not limited to, those found in Harrison's Principles of Internal Medicine, 18^{th} Edition, Eds. A. Fauci et al. McGraw-Hill N.Y., NY; Physicians Desk Reference, 65^{th} Edition, 2011, Oradell New Jersey, Medical Economics Co.; Pharmacological Basis of Therapeutics, 12^{th} Edition, Brunton et al., 2010; United States Pharmacopeia, The National Formulary, USP XXXIV NF XIX, 2011; current edition of Goodman and Gilman's The Pharmacological Basis of Therapeutics; and current edition of The Merck Index, the complete contents of all of which are incorporated herein by reference.

In some embodiments of the invention described herein, the subject is further administered a PI3K inhibitor. As used herein a “PI3K inhibitor” is any agent or compound that decreases or inhibits the activity of the PI3K protein and/or its downstream effects. PI3K inhibitors are known to those of ordinary skill in the art and can include, for example, wortmannin, LY294002, GSK 2126458, GDC-0980, GDC-0941, Sanofi XL147, XL756, XL147, PF-46915032, BKM 120, CAL-101, CAL 263, SF1126, PX-886, and a dual PI3K inhibitor (e.g., Novartis BEZ235), isoquinolinones, and INK1197 or derivatives thereof.

Efficacy

Efficacy of treatment can be assessed, for example by measuring a marker, indicator, symptom or incidence of melanoma as described herein or any other measurable parameter appropriate, e.g. tumor size. It is well within the ability of one skilled in the art to monitor efficacy of treatment or prevention by measuring any one of such parameters, or any combination of parameters.

Effective treatment is evident when there is a statistically significant improvement in one or more markers, indicators, or symptoms of melanoma, or by a failure to worsen or to develop symptoms where they would otherwise be anticipated. As an example, a favorable change of at least about 10% in a measurable parameter of melanoma, and preferably at least about 20%, about 30%, about 40%, about 50% or more can be indicative of effective treatment. Efficacy for a given inhibitor of ERK signaling and/or an activator of Wnt/β-catenin signaling or formulation of that drug can also be judged using an experimental animal model known in the art for a condition described herein. When using an experimental animal model, efficacy of treatment is evidenced when a statistically significant change in a marker is observed, e.g. the extent of the tumor or mortality.

### Subject in Need of Treatment for Melanoma

Certain aspects of the methods described herein relate to administering an inhibitor of ERK signaling and an activator of Wnt/β-catenin signaling to subjects in need of treatment for melanoma. Other aspects relate to predicting the response of a subject to an inhibitor of ERK signaling wherein the subject is in need of a treatment for melanoma. A subject in need of treatment for melanoma can be a subject having melanoma or diagnosed as having melanoma and/or at risk of having melanoma.

Subjects having melanoma can be identified by a physician using current methods of diagnosing melanoma. Excisional biopsy is the preferred diagnostic method but other types of skin biopsy can also be used including incisional biopsy, shave biopsy and punch biopsy. Metastatic melanoma may not be found until long after the original melanoma was removed from the skin. Metastatic melanoma can be diagnosed using a number of methods including fine needle aspiration biopsy, surgical lymph node biopsy and sentinel lymph node mapping and biopsy. Imaging tests such as a chest x-ray, computed tomography (CT), magnetic resonance imaging (MRI), positron emission tomography (PET) and nuclear bone scans can also be used. Melanoma is staged using the American Joint Committee on Cancer (AJCC) TNM system—Stage 0-Stage IV. The thickness of the melanoma is measured using the Breslow measurement. Invasion level is scored according to the Clark test. The extent of lymph node involvement is also an important prognostic indicator.

Symptoms of melanoma which characterize this condition and aid in diagnosis include, but are not limited to, the appearance of a new mole (which may brown, black, or have no pigment), change in the size, shape, or color of an existing mole, the spread of pigmentation beyond the border of a mole or mark, oozing or bleeding from a mole, and a mole that feels itchy, hard, lumpy, swollen, or tender to the touch.

Risk factors which can increase the likelihood of a subject being at risk of having or developing melanoma include blond or red hair, blue eyes, fair complexion, many freckles, severe sunburns as a child, family history of melanoma, dysplastic nevi (i.e., multiple atypical moles), multiple ordinary moles (>50), immune suppression, age, gender (increased frequency in men), xeroderma pigmentosum (a rare inherited condition resulting in a defect from an enzyme that repairs damage to DNA), tanning bed usage and past history of skin cancer. Intense ultraviolet light exposure is a leading risk factor for developing melanoma.

In some embodiments, subjects at risk of having or developing melanoma can be identified by measuring the levels of gene expression products of biomarkers known to be correlated with melanoma and comparing them to a reference level of those gene expression products. Examples of such biomarkers include, but are not limited to, serum levels of S100B, lactate dehydrogenase, TA901C, YKL-30, VEGF-A, CRP, IL-6, and IL-10.

### Measuring the Level of an AXIN1 Gene Product

Also provided herein are methods for determining if a subject will be responsive to treatment by an inhibitor of ERK signaling and an activator of Wnt/β-catenin signaling by determining the level of an AXIN1 protein in a sample obtained from the subject. The presence of low levels of AXIN1 protein in the sample obtained from the subject (i.e., the biological sample) can be indicate that the patient will be responsive to treatment according to the methods described herein. The level of AXIN1 protein can be determined by assessing the level in a biological sample obtained from a patient having melanoma or diagnosed as having melanoma and comparing the observed levels to the levels of AXIN1 found in a control reference sample.

As used herein, a “biological sample” refers to a sample of biological material obtained from a patient, preferably a human patient, including a tissue sample (e.g., a tissue biopsy, such as, an aspiration biopsy, a brush biopsy, a surface biopsy, a needle biopsy, a punch biopsy, an excision biopsy, an open biopsy, an incision biopsy or an endoscopic biopsy) or cell samples (e.g. epithelial cells or lymphocytes). Biological samples can also be biological fluid samples e.g. blood, serum, saliva, semen, urine, cerebrospinal fluid, and supernatant from cell lysate. Some embodiments of the present invention also encompass the use of isolates of a biological sample in the methods of the invention.

In some embodiments, the biological sample is obtained after the subject receives a dose of an inhibitor of ERK signaling and optionally, a dose of an activator of Wnt/β-catenin signaling. In these embodiments, the reference sample can be a biological sample of the subject of the same cell type taken before the subject received a dose of an inhibitor of ERK signaling.

In some embodiments, the reference value is the level of AXIN1 gene product in a control reference sample. The reference sample can be a biological sample that is obtained from melanoma cells, either from a tumor of a subject diagnosed with melanoma which is non-responsive to treatment with an inhibitor of ERK signaling and an activator of Wnt/β-catenin signaling or from melanoma cell lines that are known to be non-responsive to treatment with an inhibitor of ERK signaling and an activator of Wnt/β-catenin signaling. The control reference sample can also be a standard sample that contains essentially the same concentration of AXIN1 that is normally found in melanoma cells that are not responsive to treatment with an inhibitor of ERK signaling and an activator of Wnt/β-catenin signaling.

The reference sample can be a biological sample that is obtained from melanoma cells, either from a tumor of a subject diagnosed with melanoma or from melanoma cell lines where the subject and/or cells have not been contacted or administered an inhibitor of ERK signaling. The control reference sample can also be a standard sample that contains the same concentration of AXIN1 that is normally found in melanoma cells that have not been contacted with an inhibitor of ERK signaling.

By way of non-limiting example, there can be a standard reference control sample for the amounts of AXIN1 normally found in biological samples such as particular cell fractions, serum, blood, tumors, or skin tissue which are not responsive to treatment with an inhibitor of ERK signaling. In one embodiment, the control reference sample is a standard reference sample that contains a mean or median concentration of AXIN1 mRNA or AXIN1 protein found in melanoma cells from a population of subjects who are not responsive to treatment with an inhibitor of ERK signaling.

The level of AXIN1 protein in the biological sample is characterized as being lower than the reference value of AXIN1 gene product if the level of AXIN1 protein detected in the biological sample is lower, by a statistically significant amount, than the level of the AXIN1 protein in the reference sample. In certain embodiments, a lower level of AXIN1 in the biological sample is less than 90%, less than 80%, less than 70%, less than 60% less than 50%, less than 40%, less than 30%, less than 20%, less than 10%, or less than 5% of the reference value of AXIN1 protein. In certain embodiments, a level of AXIN1 in the biological sample that is equal to or higher than the reference value is greater than the reference value by a statistically significant amount or is not statistically different than the reference value. In certain embodiments, a higher level of AXIN1 in the biological sample is at least about 10%, at least about 30% at least about 50%, at least about 100%, at least about 200%, at least about 300% or higher than the reference value.

The levels of AXIN1 can be represented by arbitrary units, for example as units obtained from a densitometer, luminometer, or an ELISA plate reader etc.

For purposes of comparison, the biological sample and control reference sample are of the same type, that is, obtained from the same type of biological source (e.g. skin biopsies), and comprising the same composition, e.g. the same type of cells. In some embodiments, the level of AXIN1 in the samples can be normalized to the level of a gene product that is known to be relatively constant in expression, e.g. GAPDH or β-tubulin.

The levels of AXIN1, as described herein, can be measured by any means known to those of ordinary skill in the art. In certain embodiments determining of the level of AXIN1 protein involves the use of one or more of the following assays; Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; gel diffusion precipitation reaction; immunodiffusion assay; in situ immunoassay; precipitation reaction; agglutination assay; complement fixation assay; immunofluoresence assay; mass spectroscopy and/or immunoelectrophoresis assay. In certain embodiments determining of the level of AXIN1 protein involves the use of an antibody, an antibody fragment, a monoclonal antibody, a monoclonal antibody fragment, a protein binding protein, and/or an AXIN1-binding peptide.

AXIN1 protein levels can also be measured, in particular, when the biological sample is a fluid sample such as cell lysate. In one embodiment, levels of AXIN1 protein are measured by contacting the biological sample with an antibody moiety that specifically binds to AXIN1, or to a fragment of AXIN1. Formation of the antibody-AXIN1 complex is then detected as a measure of AXIN1 levels. Antibodies which recognize AXIN1 can be obtained commercially (e.g., ab56475, AbCam Cambridge, Mass.) or prepared according to the methods known in the art or described elsewhere herein.

In one embodiment, the antibody moiety is detectably labeled. “Labeled antibody”, as used herein, includes antibodies that are labeled by a detectable means and include, but are not limited to, antibodies that are enzymatically, radioactively, fluorescently, and/or chemiluminescently labeled. Antibodies can also be labeled with a detectable tag, such as c-Myc, HA, VSV-G, HSV, FLAG, V5, or HIS. In the diagnostic and prognostic methods described herein that use antibody based binding moieties for the detection of AXIN1, the level of AXIN1 present in the biological samples correlate to the intensity of the signal emitted from the detectably labeled antibody. In one preferred embodiment, the antibody-based binding moiety is detectably labeled by linking the antibody to an enzyme. The enzyme, in turn, when exposed to its substrate, will react with the substrate in such a manner as to produce a chemical moiety which can be detected, for example, by spectrophotometric, fluorometric or by visual means. Enzymes which can be used to detectably label antibodies include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, β-galactosidase, ribonuclease, urease, catalase, glucose-VI-phosphate dehydrogenase, glucoamylase and acetylcholinesterase.

Detection can also be accomplished using any of a variety of other immunoassays. For example, by radioactively labeling an antibody, it is possible to detect the antibody through the use of radioimmunology assays. The radioactive isotope can be detected by such means as the use of a gamma counter or a scintillation counter or by autoradiography. Isotopes which are particularly useful for the purpose of the present invention are ³H, ¹³¹I, ³⁵S, ¹⁴C, and preferably ¹²⁵I. It is also possible to label an antibody with a fluorescent compound. When the fluorescently labeled antibody is exposed to light of the proper wavelength, its presence can then be detected due to fluorescence. Among the most commonly used fluorescent labeling compounds are CYE dyes, fluorescein isothiocyanate, rhodamine, phycoerytherin, phycocyanin, allophycocyanin, o-phthaldehyde and fluorescamine. An antibody can also be detectably labeled using fluorescence emitting metals such as ¹⁵²Eu, or others of the lanthanide series. These metals can be attached to the antibody using such metal chelating groups as diethylenetriaminepentaacetic acid (DTPA) or ethylenediaminetetraacetic acid (EDTA). An antibody also can be detectably labeled by coupling it to a chemiluminescent compound. The presence of the chemiluminescent-antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

In one embodiment, the level of AXIN1 protein is detected by immunoassay, such as an enzyme linked immunoabsorbant assay (ELISA), Western blotting, immunocytochemistry or flow cytometry. Immunoassays such as ELISA, flow cytometry or RIA, can be extremely rapid. Antibody arrays or protein chips can also be employed, see for example U.S. Patent Application Nos: 20030013208A1; 20020155493A1; 20030017515 and U.S. Pat. Nos. 6,329,209; 6,365,418, which are herein incorporated by reference in their entirety.

The most common enzyme immunoassay is ELISA, which is a technique for detecting and measuring the concentration of an antigen using a labeled (e.g. enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904.

Other antibody-based detection methods are known to those of skill in the art and include, for example, gel precipitation assay, radial immunodiffusion, double diffusion gel precipitation and Ouchterlony double immunodiffusion. Quantitative precipitation assays are known to those skilled in the art (Basic Techniques in Biochemistry and Molecular Biology. Sharma and Sangha (Eds), I.K. International Publishers Pvt. Lt, New Delhi, India (2009); Essentials of Immunology and Serology. Stanley, J. Thomson, Albany, N.Y., (2002)). Other assays well known in the art include radioimmunoassay, Western blotting, or mass spectroscopy (MS, including, e.g., MADLI/TOF, SELDI/TOF, LC-MS, GC-MS, HPLC-MS, etc., among others).

### Measuring the Level of a Marker of Nuclear β-Catenin

Also provided herein are methods for determining if a subject will be responsive to treatment by an inhibitor of ERK signaling and optionally an activator of Wnt/β-catenin signaling by determining the level of a marker of nuclear β-catenin in a sample obtained from the subject. As noted herein above, activation of Wnt/β-catenin signaling induces the tranlocation of β-catenin to the nucleus. The presence of low levels of a marker of nuclear β-catenin in the sample obtained from the subject (i.e., the biological sample) is indicative that the patient will not be responsive or will be less responsive to treatment according to treatment with an ERK inhibitor but that sensitivity can be restored or established by activating Wnt/β-catenin signaling. The level of a marker of nuclear β-catenin (e.g. mRNA or protein) can be determined by assessing the level in a biological sample obtained from a patient having melanoma or diagnosed as having melanoma and comparing the observed levels to the levels of a marker of nuclear β-catenin found in a control reference sample.

As used herein, a “marker of nuclear β-catenin” or a “nuclear β-catenin marker” can be a protein or mRNA. A marker of nuclear β-catenin can be β-catenin which is localized to the nucleus, or dephosphorylated β-catenin, or mRNA or protein encoded by a gene whose transcription is increased by nuclear β-catenin. Examples of markers of nuclear β-catenin, include, but are not limited to the gene products (i.e. the mRNA transcript or protein) of FZD7 (NCBI Gene ID No: 8324); LEF1 (NCBI Gene ID No: 51176); AXIN2 (NCBI Gene ID No: 8313); DKK1 (NCBI Gene ID No: 22943); DKK2 (NCBI Gene ID No: 27123); DKK3 (NCBI Gene ID No: 27122); DKK4 (NCBI Gene ID No: 27121); FN1 (NCBI Gene ID No: 2335); TCF7 (NCBI Gene ID No: 6932); MYCN (NCBI Gene ID No: 4613); MYC (NCBI Gene ID No: 4609); SNAI1 (NCBI Gene ID No: 6815); LGR5 (NCBI Gene ID No: 8549); LBH (NCBI Gene ID No: 81606); FGF9 (NCBI Gene ID No: 2254); POU5F (NCBI Gene ID No: 5460); CYR61 (NCBI Gene ID No: 3491); GREM1 (NCBI Gene ID No: 26585); RUNX2 (NCBI Gene ID No: 860); SOX17 (NCBI Gene ID No: 64321); ISL1 (NCBI Gene ID No: 3670); FST (NCBI Gene ID No: 10468); NOS2 (NCBI Gene ID No: 4843); JAG1 (NCBI Gene ID No: 182); ID2 (NCBI Gene ID No: 3398); L1CAM (NCBI Gene ID No: 3897); MYCBP (NCBI Gene ID No: 26292); EDN1 (NCBI Gene ID No: 1906); MET (NCBI Gene ID No: 4233); FGF18 (NCBI Gene ID No: 8817); VEGFA (NCBI Gene ID No: 7422); BIRC5 (NCBI Gene ID No: 332); CLDN1 (NCBI Gene ID No: 9076); BMP4 (NCBI Gene ID No; 652); CD44 (NCBI Gene ID No: 960); GAST (NCBI Gene ID No: 2520); TCF4 (NCBI Gene ID No: 6925); NRCAM (NCBI Gene ID No: 4897); MMP7 (NCBI Gene ID No: 4316); PLAUR (NCBI Gene ID No: 5329); FOSL1 (NCBI Gene ID No: 8061); JUN (NCBI Gene ID No: 3725); PPARD (NCBI Gene ID No: 5467) and CTLA4 (NCBI Gene ID No: 1493). An increase in such a marker over background can be indicative of nuclear β-catenin or Wnt/β-catenin signaling activity.

In some embodiments, nuclear β-catenin can be measured directly by visualization of β-catenin in the nucleus versus the cytoplasm of cells in a cell culture or tissue sample. The following method can be used. A polyclonal rabbit anti-β-catenin antibody (Sigma, Cat# C2206) is used for detection of β-catenin (1:1000 dilution for immunoblot, 1:200 dilution for immunohistochemistry). Cell grown on 18 mm glass coverslips for 48-72 hours, or, alternatively, sections of a tissue sample, are fixed using 4% paraformaldahyde, permeabilized using 0.25% Triton X-100, and then blocked with 10% goat serum. Goat anti-rabbit Alexa Fluor-568 antibody (Molecular Probes; Eugene, Oreg.) is diluted 1:1000. Cells are counterstained for nucleic acid with DAPI (Molecular Probes; Eugene, Oreg.). Automated quantitative analysis (AQUA®) is then used to measure levels of nuclear β-catenin (Camp et al., “Automated Subcellular Localization and Quantification of Protein Expression in Tissue Microarrays,” Nat Med 8:1323-7 (2002), which is hereby incorporated by reference in its entirety). Labeling with 4′,6-diamidino-2-phenylindole (DAPI) can be used to define nuclei. This method allows for clear distinction between nuclear and cytoplasmic/membranous β-catenin. Additional methods are known to those of ordinary skill in the art.

In some embodiments, the reference value is the level of the gene product of a marker of nuclear β-catenin in a control reference sample. The reference sample can be from a cell type that is known to have low levels of nuclear β-catenin and/or low levels of Wnt/β-catenin signaling. By way of non-limiting example, cell types which are particularly useful in the methods described herein which have low levels of nuclear β-catenin include, but are not limited to, cells which have been contacted with RNAi to specifically deplete β-catenin or human H1 embryonic stem cells. The control reference sample can also be a standard sample that contains the same concentration of the gene product of a marker of nuclear β-catenin that is normally found in cells with low levels of nuclear β-catenin.

By way of non-limiting example, there can be a standard reference control sample for the amounts of a gene product of a marker of nuclear β-catenin that is normally found in biological samples such as particular cell fractions, serum, blood, tumors, or skin tissue which have low levels of nuclear β-catenin and/or low levels of Wnt/β-catenin signaling. In one embodiment, the control reference sample is a standard reference sample that contains a mean or median concentration of a gene product of a marker found in cells which have low levels of nuclear β-catenin and/or low levels of Wnt/β-catenin signaling.

The level of gene product of a marker of nuclear β-catenin in the biological sample is characterized as being greater than the reference value of the gene product if the level of the gene product detected in the biological sample is greater, by a statistically significant amount, than the level detected in the reference sample. In certain embodiments, a greater level of gene product of a marker of nuclear β-catenin in the biological sample is more than 10%, more than 20%, more than 30%, more than 50%, more than 75%, more than 100%, more than 200%, or more than 300% of the reference value of a marker of nuclear β-catenin.

The level of gene product of a marker of nuclear β-catenin in the biological sample is characterized as being less than the reference value of the gene product if the level of the gene product detected in the biological sample is less, by a statistically significant amount, than the level detected in the reference sample. In certain embodiments, a lower level of gene product of a marker of nuclear β-catenin in the biological sample is 95% of or less, 90% of or less, 80% of or less, 70% of or less, 60% of or less, 50% of or less, 40% of or less, 30% of or less, 20% of or less, or 10% of or less than the reference value of a marker of nuclear β-catenin.

The levels of a marker of nuclear β-catenin can be represented by arbitrary units, for example as units obtained from a densitometer, luminometer, or an ELISA plate reader etc.

For purposes of comparison, the biological sample and control reference sample can be of the same type, that is, obtained from the same type of biological source (e.g. skin biopsies), and comprising the same composition, e.g. the same type of cells. In some embodiments, the level of gene product of a marker of nuclear β-catenin in the samples can be normalized to the level of a gene product that is known to be relatively constant in expression, e.g. GAPDH or β-tubulin.

The levels of gene product of a marker of nuclear β-catenin, as described herein, can be measured by any means known to those of ordinary skill in the art.

In certain embodiments, the determination of the level of a marker of nuclear β-catenin which is an mRNA involves the use of one or more of the following assays: RT-PCR; quantitative RT-PCR; RNA-Seq; Northern blo; microarray based expression analysis; transcription amplification and/or self-sustained sequence replication.

Methods for assessing levels of mRNA are well known to those skilled in the art and any suitable method can be used. In one embodiment a tumor sample or biopsy is obtained and Laser Capture Microdissection (LCM) (see, for example, Simon et al. (1998) Trends in Genetics 14:272 and Emmert-Buck et al. (1996) Science 274:998-1001) is used to obtain genetic material, such as, mRNA, for analysis. RNA molecules can be isolated from a particular biological sample using any of a number of procedures, which are well known in the art, the particular isolation procedure chosen being appropriate for the particular biological sample.

Detection of RNA transcripts can further be accomplished using known amplification methods. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap lipase chain reaction (RT-AGLCR) as described by R. L. Marshall, et al., PCR Methods and Applications 4: 80-84 (1994). Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO 9322461; “self-sustained sequence replication” As described in Guatelli, et al., Proc. Nat. I. Acad. Sci. USA 87:1874 (1990); or “transcription amplification” as described in Kwoh (1989) Proc. Natl. Acad. Sci. USA 86: 1173.

As but one example of an amplification based assay for RNA levels, real time PCR can be used (see, e.g., Gibson et al., Genome Research 6:995-1001, 1996; Heid et al., Genome Research 6:986-994, 1996). Real-time PCR evaluates the level of PCR product accumulation during amplification. This technique permits quantitative evaluation of mRNA levels in multiple samples. For mRNA levels, mRNA is extracted from a biological sample, e.g. a tumor and normal tissue, and cDNA is prepared using standard techniques. Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, β-actin) primers and probes may be obtained commercially from, for example, Perkin Elmer/Applied Biosystems (Foster City, Calif.).

Quantitative PCR methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided, for example, in Innis et al. (1990) PCR Protocols, A Guide to Methods and Applications, Academic Press, Inc. N.Y. To measure the amount of the specific nucleic acid of interest in a sample, a standard curve is generated using a control. Standard curves can be generated using the Ct values determined in the real-time PCR, which are related to the initial concentration of the nucleic acid of interest used in the assay. Standard dilutions ranging from 10-10⁶ copies of the gene of interest are generally sufficient. In addition, a standard curve is generated for the control sequence. This permits standardization of initial content of the nucleic acid of interest in a tissue sample to the amount of control for comparison purposes. Methods of real-time quantitative PCR using TaqMan probes are well known in the art. Detailed protocols for real-time quantitative PCR are provided, for example, for RNA in: Gibson et al., 1996 Genome Res., 10:995-1001; and for DNA in: Heid et al., 1996 Genome Res., 10:986-994.

A TaqMan-based assay also can be used to quantify polynucleotides. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification.

In another embodiment, for example, detection of RNA transcripts can be achieved by Northern blotting, wherein a preparation of RNA is separated on a denaturing agarose gel, and transferred to a suitable support, such as activated cellulose, nitrocellulose or glass or nylon membranes. Labeled (e.g., radiolabeled) cDNA or RNA is then hybridized to the preparation, washed and analyzed by methods such as autoradiography.

In situ hybridization visualization can also be employed, wherein a radioactively labeled antisense RNA probe is hybridized with a thin section of a biopsy sample, washed, cleaved with RNase and exposed to a sensitive emulsion for autoradiography. The samples can be stained with hematoxylin to demonstrate the histological composition of the sample, and dark field imaging with a suitable light filter shows the developed emulsion. Non-radioactive labels such as digoxigenin can also be used.

Alternatively, mRNA expression can be detected on a DNA array, chip or a microarray. Oligonucleotides corresponding to the mRNA which is a marker of nuclear β-catenin are immobilized on a chip which is then hybridized with labeled nucleic acids of a test sample obtained from a subject. Positive hybridization signal is obtained with the sample containing transcripts which are markers of nuclear β-catenin. Methods of preparing DNA arrays and their use are well known in the art. (See, for example U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). To monitor mRNA levels, for example, mRNA is extracted from the biological sample to be tested, reverse transcribed, and fluorescent-labeled cDNA probes are generated. The microarrays capable of hybridizing to cDNA of a marker of nuclear β-catenin are then probed with the labeled cDNA probes, the slides scanned and fluorescence intensity measured. This intensity correlates with the hybridization intensity and expression levels.

Detection of a marker of nuclear β-catenin can also rely upon detection of proteins. Protein detection methods are well known to those of ordinary skill in the art and are described herein above in relation to methods of measuring AXIN1 polypeptides. Thus, the level of a marker of nuclear β-catenin which is a protein can be measured then, according to any of the methods described in the section entitled “Measuring the level of an AXIN1 Gene Product.” Antibodies which recognize markers of nuclear β-catenin can be obtained commercially, for example, antibodies to β-catenin (ab32572; AbCam Cambridge, Mass.) or prepared according to the methods described elsewhere herein.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); The ELISA guidebook (Methods in molecular biology 149) by Crowther J. R. (2000); Fundamentals of RIA and Other Ligand Assays by Jeffrey Travis, 1979, Scientific Newsletters; Immunology by Werner Luttmann, published by Elsevier, 2006. Definitions of common terms in molecular biology are also be found in Benjamin Lewin, Genes IX, published by Jones & Bartlett Publishing, 2007 (ISBN-13: 9780763740634); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Methods in Enzymology, Volume 289: Solid-Phase Peptide Synthesis, J. N. Abelson, M. I. Simon, G. B. Fields (Editors), Academic Press; 1st edition (1997) (ISBN-13: 978-0121821906); U.S. Pat. Nos. 4,965,343, and 5,849,954; Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

The inventors, as described herein, have discovered that the ERK signaling pathway, which is constitutively activated in many melanomas by the BRAF^{V600E} mutation, negatively regulates Wnt/β-catenin signaling in human melanoma cells. As inhibitors of BRAF^{V600E} show promise in ongoing clinical trials, the inventors determined whether altering Wnt/β-catenin signaling might enhance the efficacy of a BRAF^{V600E} inhibitor. Surprisingly, endogenous β-catenin is required for the BRAF^{V600E} inhibitor to induce apoptosis in melanoma cells, while activation of Wnt/β-catenin signaling strongly synergizes with the BRAF^{V600E} inhibitor to decrease tumor growth in vivo and to increase apoptosis in vitro. This synergistic enhancement of apoptosis correlates with a reduction in the steady-state levels of a β-catenin antagonist, AXIN1. In support of the hypothesis that AXIN1 is a mediator rather than marker of apoptosis, melanoma cell lines that are resistant to apoptosis after treatment with a BRAF^{V600E} inhibitor become susceptible, and undergo apoptosis, when levels of AXIN1, but not AXIN2, are reduced by siRNA. These findings point to a significant role for Wnt/β-catenin signaling and AXIN1 in regulating the efficacy of inhibitors of BRAF^{V600E}, and lay a novel foundation for combination therapies and biomarkers.

### Example 1

### Wnt/β-Catenin Signaling and AXIN1 Regulate Apoptosis Mediated by Inhibition of BRAF^{V600E} Kinase in Human Melanoma

A High-Throughput Screen Identifies BRAF and MEK as Negative Regulators of Wnt/β-Catenin Signaling in Melanoma Cells but not Melanocytes.

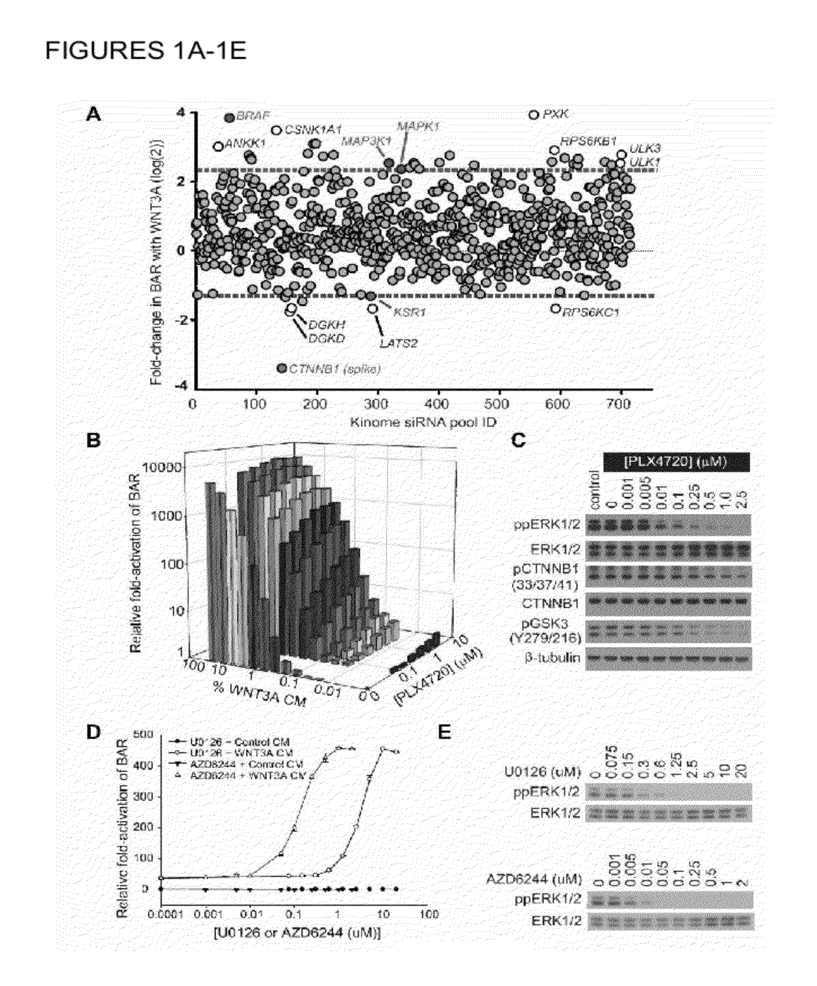

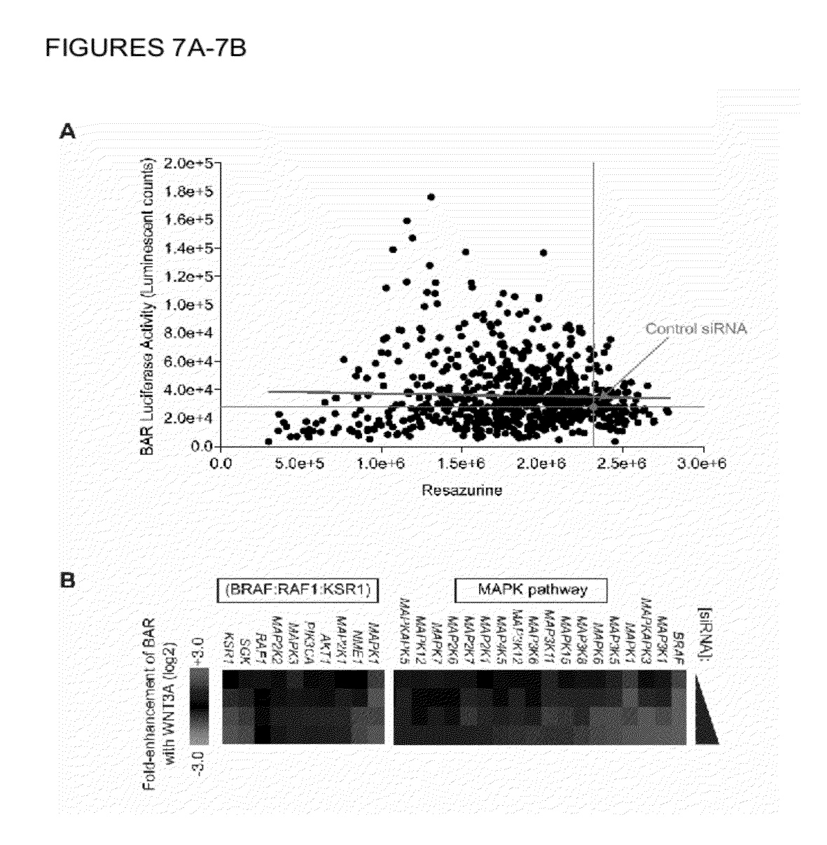

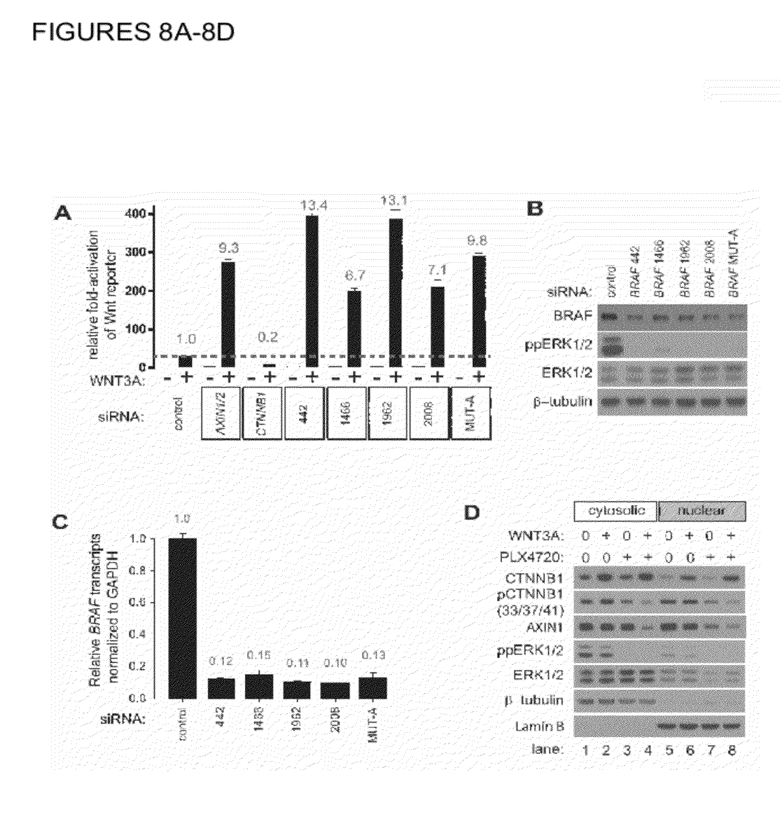

To identify regulators of Wnt/β-catenin signaling in melanoma, A375 human melanoma cells (which harbor the BRAF^{V600E} mutation) stably expressing a β-catenin-activated reporter (BAR) were employed in a high-throughput siRNA screen targeting 716 genes encoding known or predicted kinases. Cells transfected with four different concentrations of siRNA were treated with an EC₂₀ dose of Wnt3a conditioned media. Reporter activity was normalized to cell viability, and then fold-activation was compared to control siRNA. This screen revealed the BRAF siRNAs strongly synergize with WNT3A to activate the BAR reporter (FIG. 1A and FIGS. 7A-B). The gene exhibiting the highest synergy with Wnt3A upon siRNA knockdown is BRAF, implying that activated BRAF^{V600E} inhibits Wnt/β-catenin signaling (FIG. 1A). This result was validated with four independent siRNAs targeting BRAF as well as with a published siRNA that specifically targets activated BRAF^{V600E} (24). These data support the unexpected hypothesis that activated BRAF^{V600E} negatively regulates Wnt/β-catenin signaling in melanoma (FIG. 8A-C and Table 1).

The presence of the BRAF^{V600E} mutant kinase results in downstream activation of the ERK signaling pathway, and notably several other members of the ERK pathway exhibit dose-dependent activation of Wnt/β-catenin signaling upon siRNA knockdown (FIG. 7B). Analysis of proteins described in the literature to complex with BRAF revealed no effect of RAF1 (C-RAF) knockdown (FIG. 7B). Interestingly, genes encoding the kinase SGK1 and the scaffolding protein KSR1, a known regulator of ERK signaling, are both required for Wnt/β-catenin signaling in this screen (FIG. 7B). The regulation of Wnt/β-catenin signaling by BRAF was validated with five independent siRNAs (FIG. 8A). Activation of BRAF leads to the downstream phosphorylation of MEK, which subsequently phosphorylates and activates ERK1/2. Consequently, the loss of BRAF would be expected to result in a loss of phosphorylated ERK1/2 (p-ERK1/2). Interestingly, although BRAF protein levels were reduced but readily detectable by immunoblot (FIG. 8B), near-total inhibition of BRAF transcript was observed by quantitative real-time PCR (qPCR; data not shown) and nearly complete loss of ERK phosphorylation (FIG. 8B) was demonstrated, indicating that the knockdown of BRAF was functionally sufficient and relevant.

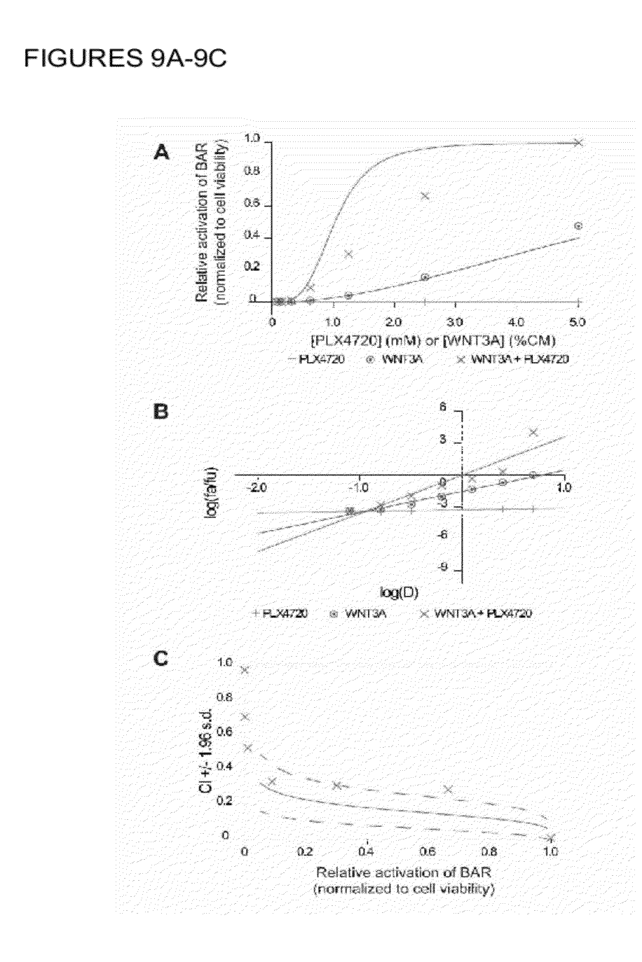

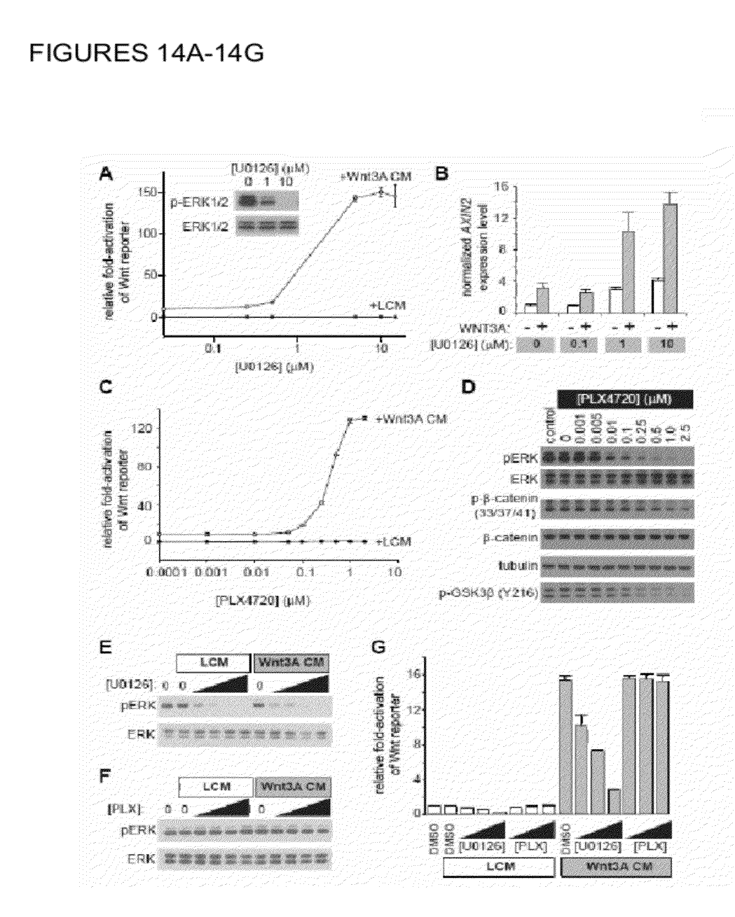

Next, whether the enhancement of Wnt/β-catenin signaling observed with BRAF siRNAs could be phenocopied with PLX4720, a small molecule designed to specifically inhibit the constitutively-active BRAF^{V600E} mutant kinase (2) was examined. PLX4720 enhanced Wnt/β-catenin signaling in a dose-dependent manner (FIG. 1B, FIG. 9A-9B, FIG. 14C), corresponding with the dose-dependent inhibition of dual-phosphorylated ERK1/2 (ppERK1/2) (FIG. 1C, FIG. 14D). Combination indices for WNT3A and PLX4720 were much less than 1, particularly at higher levels of BAR activity (FIG. 9C), supporting a synergistic interaction between these two drugs with respect to Wnt/β-catenin activation. In further support of a synergistic interaction, the addition of PLX4720 led to a calculated WNT3A dose-reduction index of 6.0 at the level of BAR response corresponding to the EC₅₀ for WNT3A alone.

PLX4720 treatment decreased phosphorylation of β-catenin at sites normally phosphorylated by glycogen synthase kinase-3β (GSK3β/GSK3B) to target β-catenin for proteasomal degradation (FIG. 1C, FIG. 14D). Consistent with this observation, the activating auto-phosphorylation of GSK3B at Tyr216 was lost upon treatment of cells with PLX4720 (FIG. 1C). Similarly, treatment of A375 melanoma cells with either PLX4720 (FIG. 14D) or U0126 (data not shown) results in the loss of the activating auto-phosphorylation of GSK3B at Tyr117. While these effects on phosphorylation of β-catenin and GSK3B did not result in increased steady-state levels of cytosolic or nuclear β-catenin (FIG. 8D), recent findings have established that activation of β-catenin function in melanoma correlates with the same changes in phosphorylation shown herein (FIG. 1C) rather than with changes in steady-state levels of β-catenin (25). As BRAF signals through the downstream kinase MEK, the effects on β-catenin signaling of two independent small molecule MEK inhibitors, U0126 and AZD6244 (26) were examined. Both drugs synergistically enhanced Wnt/β-catenin activation as measured by the BAR assay (FIG. 1D), and did so at doses that correlate with the inhibition of ERK phosphorylation (FIG. 1E). These results solidify the role of the BRAF/MEK/ERK cascade as a regulator of Wnt/β-catenin signaling in melanoma.

Consistent with these results, treatment of A375 melanoma cells with the MEK inhibitor U0126 leads to a dose-dependent increase in stimulated Wnt/β-catenin signaling as measured by either the BAR reporter (FIG. 14A) or by levels of the endogenous Wnt/β-catenin target gene AXIN2 (FIG. 14B), supporting a model in which BRAF^{V600E} negatively regulates Wnt/β-catenin signaling in melanoma cells through the downstream effector MEK. In parallel, decreased phosphorylation of β-catenin at sites normally regulated by glycogen synthase kinase-3β (GSK3B) to target β-catenin for ubiquitination and proteasomal degradation with U0126 (data not shown) were observed.

While p-ERK is seen in cultured primary human epidermal melanocytes (HEM), mutations of BRAF in this context have not been reported. Consistent with these observations, U0126 treatment inhibits ERK1/2 phosphorylation in a dose-dependent manner in HEMs (FIG. 14E), while there is no effect of BRAF^{V600E} inhibition by PLX4720 on ERK1/2 phosphorylation (FIG. 14F). In contrast to the enhancement of Wnt/β-catenin signaling upon MAPK/ERK inhibition in melanoma cells, treatment with U0126 actually inhibited Wnt/β-catenin signaling in HEMs (FIG. 14G). As predicted by its lack of effect on p-ERK levels, PLX4720 also had no detectable effect on Wnt/β-catenin signaling in HEMs (FIG. 14G).

WNT3A Enhances the Ability of an Inhibitor of BRAF^{V600E} to Reduce Tumor Size

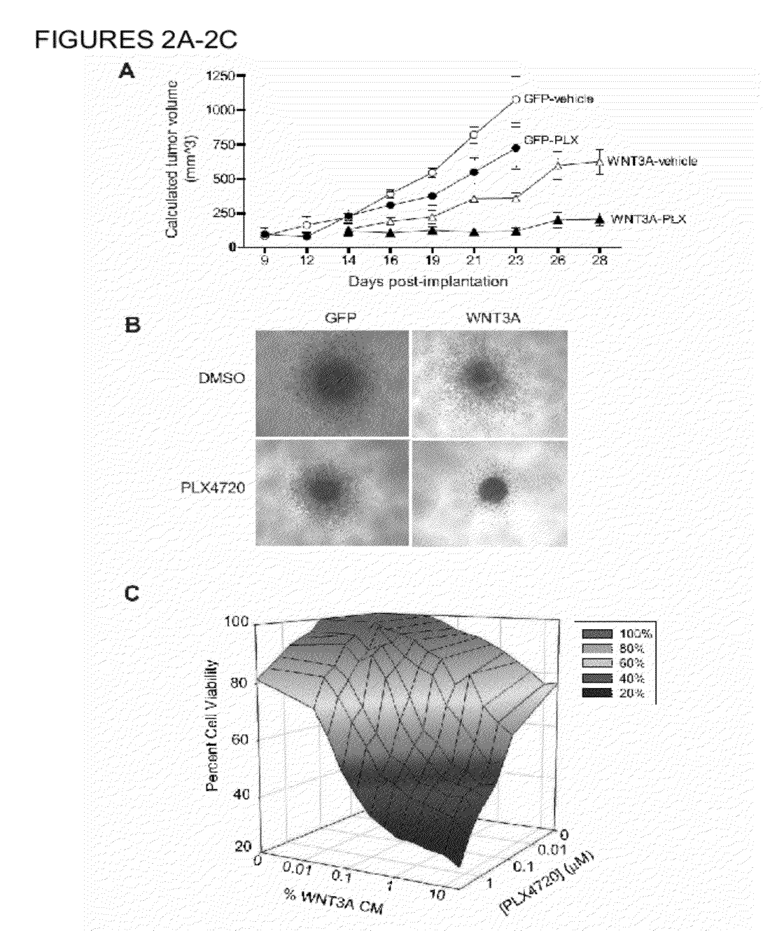

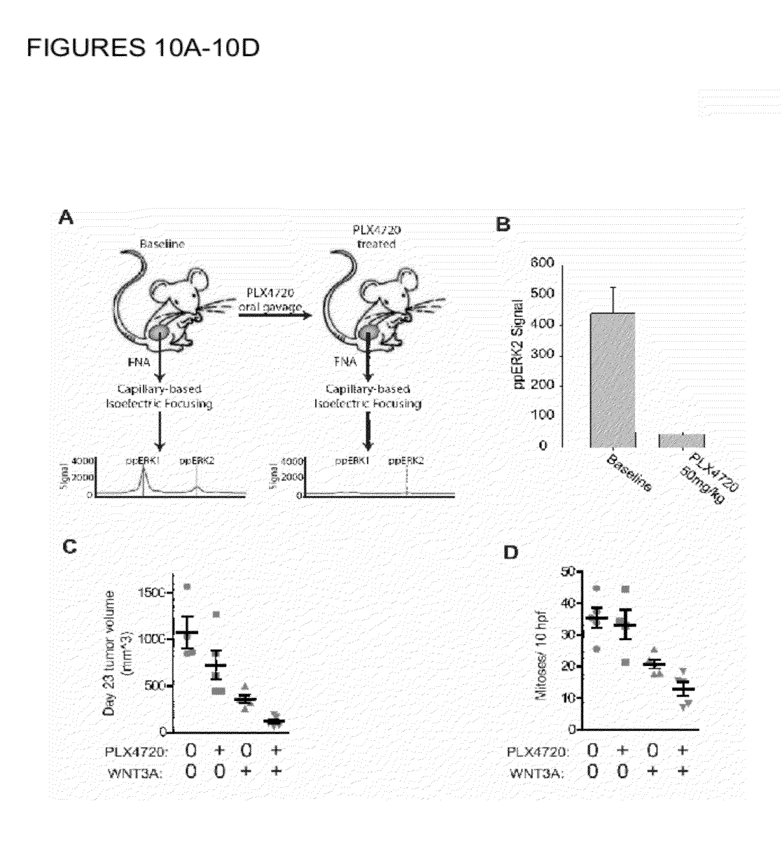

It was next asked whether combined inhibition of BRAF^{V600E} and activation of Wnt/β-catenin signaling would cooperate to reduce tumor size. Immunosuppressed mice harboring subcutaneous xenografts generated from human A375:GFP cells (controls) or A375:WNT3A cells (expressing WNT3A-iresGFP) were treated by oral gavage with either vehicle or PLX4720. Inhibition of ppERK1/2 in vivo following PLX4720 treatment was confirmed using biochemical analysis of fine-needle aspirates sampled from tumors during treatment (FIG. 10A-10B). Results of the xenograft study revealed that treatment of A375:GFP tumors with PLX4720 decreased tumor growth compared to drug vehicle (FIG. 2A). The growth of A375:WNT3A tumors was slower compared to both A375:GFP tumors treated with vehicle and A375:GFP tumors treated with PLX4720. Remarkably, the effects of PLX4720 on A375:WNT3A tumor growth were even more pronounced than the effects on A375:GFP tumors, with near complete suppression of A375:WNT3A tumor growth over four weeks. Growth curves were significantly different upon one-way ANOVA with a post-test for linear trend (p=0.024). Direct comparisons of tumor volume between groups at day 23 (FIG. 10C) revealed a highly significant difference upon one-way ANOVA with post-test for linear trend (p<0.0001). These results paralleled the significant differences seen in mitotic index (p<0.0001) upon histological analysis of the xenografts (FIG. 10D). This indicates that WNT3A and PLX4720 act synergistically to reduce melanoma tumor size in this xenograft assay.

To confirm and extend these results a three-dimensional spheroid assay of tumor cell growth and invasion within a collagen matrix was utilized. Treatment of both A375:GFP- and A375:WNT3A-derived spheroids with PLX4720 decreased spheroid size (FIG. 2B), paralleling the decreased tumor sizes observed in xenograft studies (FIG. 2A). Treatment of spheroids expressing WNT3A with PLX4720 led to a dramatic decrease in the number of invasive cells at 72 hours compared to either A375:WNT3A-derived spheroids treated with DMSO or to A375:GFP-derived spheroids treated with PLX4720 (FIG. 2B).

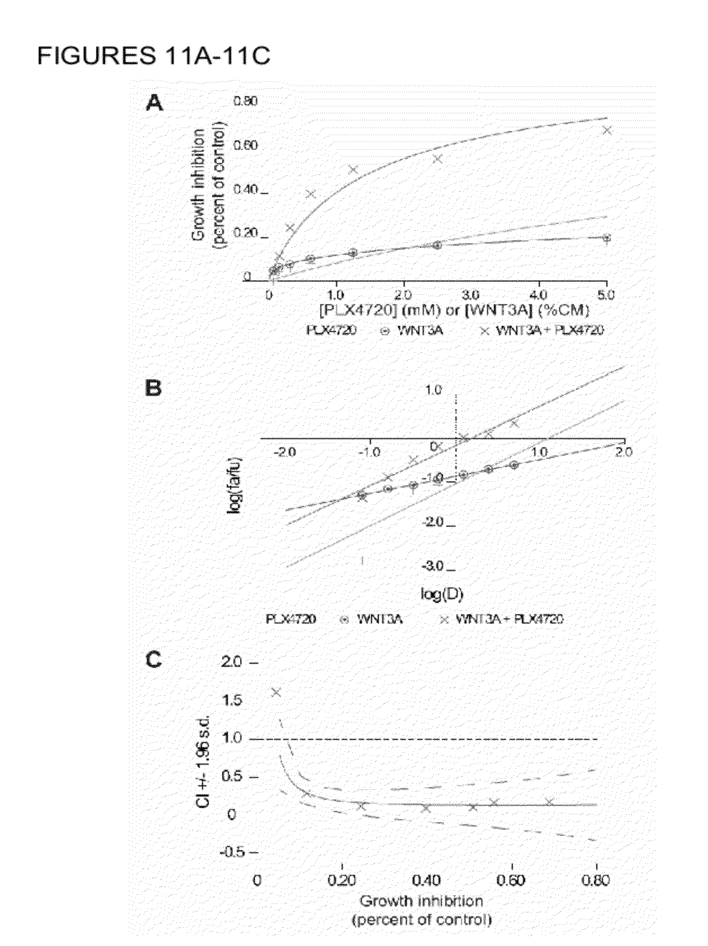

Synergistic inhibition of melanoma cell growth by WNT3A and PLX4720 was next tested in two-dimensional cell culture. Cell viability was measured in A375 melanoma cells treated with combinations of WNT3A and PLX4720 at various concentrations (FIG. 2C and FIGS. 11A-11B). The inhibition of cell growth by the combination of WNT3A and PLX4720 resulted in combination indices much less than 1 (FIG. 11C). At 50% growth inhibition, the drug reduction indices were 8.1 for PLX4720 and 117.4 for WNT3A CM, further supporting a synergistic effect of these two drugs. Together, these studies using three different assays demonstrate that the simultaneous activation of Wnt/β-catenin signaling and the targeted inhibition of BRAF^{V600E} by PLX4720 functionally cooperate to decrease melanoma cell growth both in vivo and in vitro.

WNT3A Enhances the Ability of an Inhibitor of BRAF^{V600E} to Increase Apoptosis

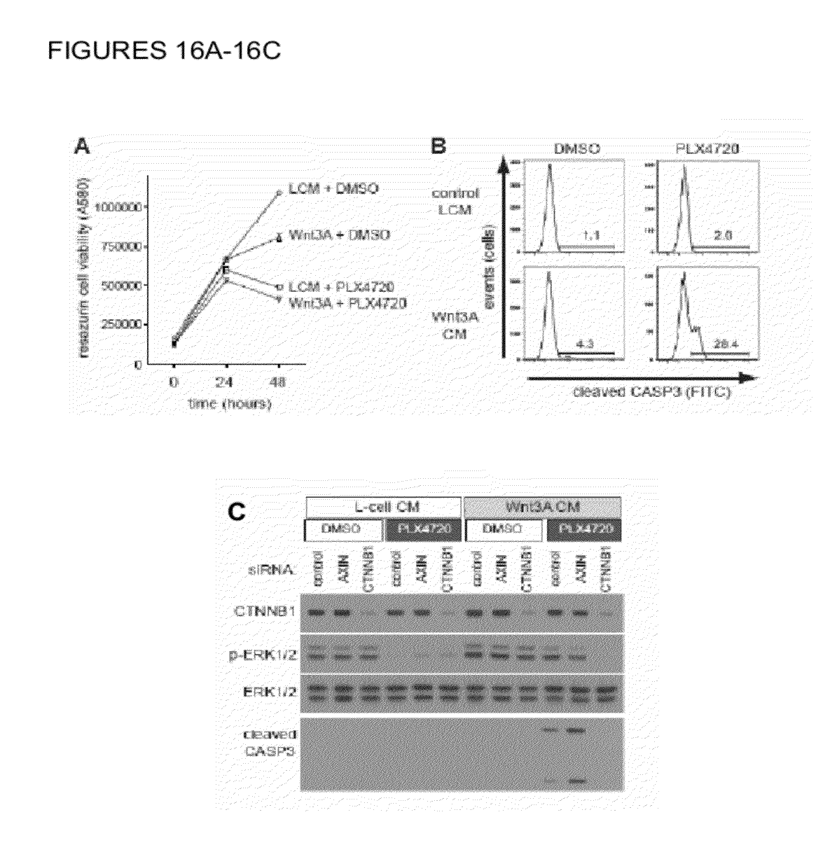

The inventors have previously shown that forced expression of Wnt3A decreases the proliferation of melanoma cells both in vitro and in vivo. The data above establish that inhibition of BRAF^{V600E} reduces tumor size, and that such treatments act synergistically with activation of Wnt/β-catenin signaling. Whether this reduction in tumor size was the consequence of cell death was examined next. Using a resazurin-based assay for cell viability, a similar decrease in proliferation is observed in human A375 melanoma cells treated with Wnt3A conditioned media (CM) compared to cells treated with control L-cell CM (FIG. 16A). The addition of PLX4720 further decreases cell viability in the absence and presence of Wnt3A CM, including a marked drop in cell viability at 48 hours after treatment (FIG. 16A). Visual appraisal of cells treated with Wnt3a and PLX4720 suggested the presence of dying cells.

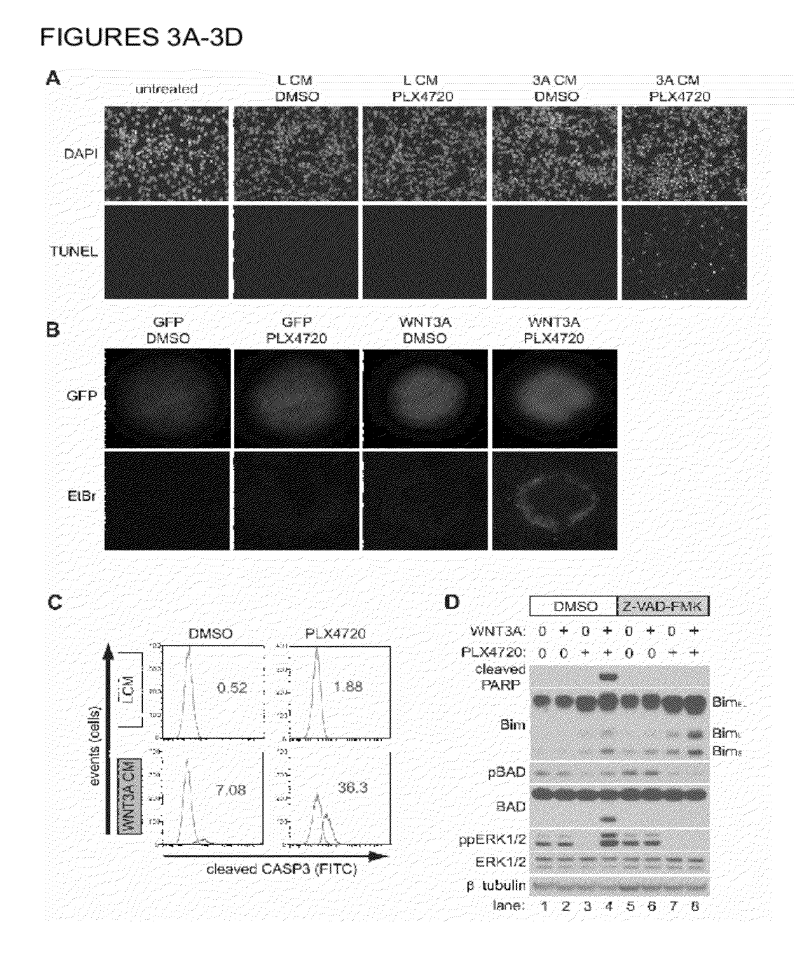

TUNEL staining of melanoma cells treated for 24 hours with WNT3A and PLX4720 indicated the presence of apoptotic cell death (FIG. 3A), a finding consistent with the detection of dead cells only in A375 spheroids concurrently expressing WNT3A and treated with PLX4720 (FIG. 3B). These findings were confirmed by flow cytometry using an antibody that detects the cleaved (active) form of caspase-3 (FIG. 3C; FIG. 16B). Consistent with the TUNEL and the spheroid assays, no apoptosis was seen in the presence of DMSO vehicle alone, and minimal increases in cleaved caspase-3 were seen with either PLX4720 or WNT3A conditioned media (CM) alone. However, in the presence of both WNT3A CM plus PLX4720, cleaved caspase-3 increased ˜5-20-fold (FIG. 3C). In support of a caspase-mediated apoptotic pathway, combined treatment with WNT3A and PLX4720 led to synergistic cleavage of the caspase-3 substrate, PARP1 (FIG. 3D lane 4 versus lane 2 and 3). This PARP1cleavage was completely abolished by addition of the pan-caspase inhibitor, Z-VAD-FMK (FIG. 3D, lane 8 versus lane 4).

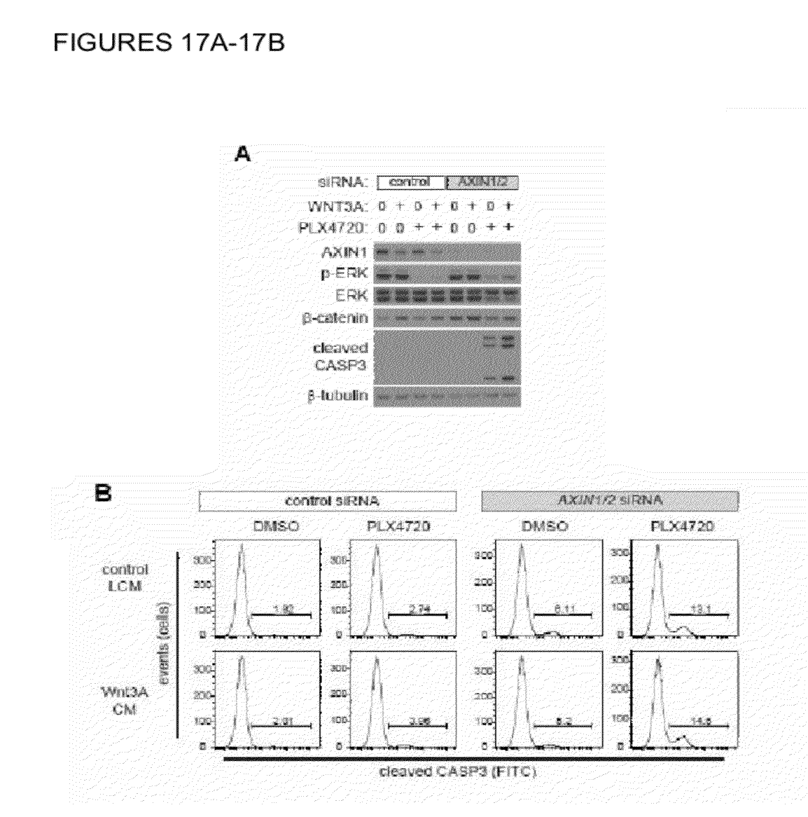

In the previous experiments, exogenous WNT3A was added to induce apoptosis in the presence of PLX4720. Longer treatment of A375 melanoma cells with PLX4720 led to activation of caspase3 and importantly, this caspase3 activation was blocked by siRNA mediated knockdown of β-catenin (FIGS. 17A-17B).

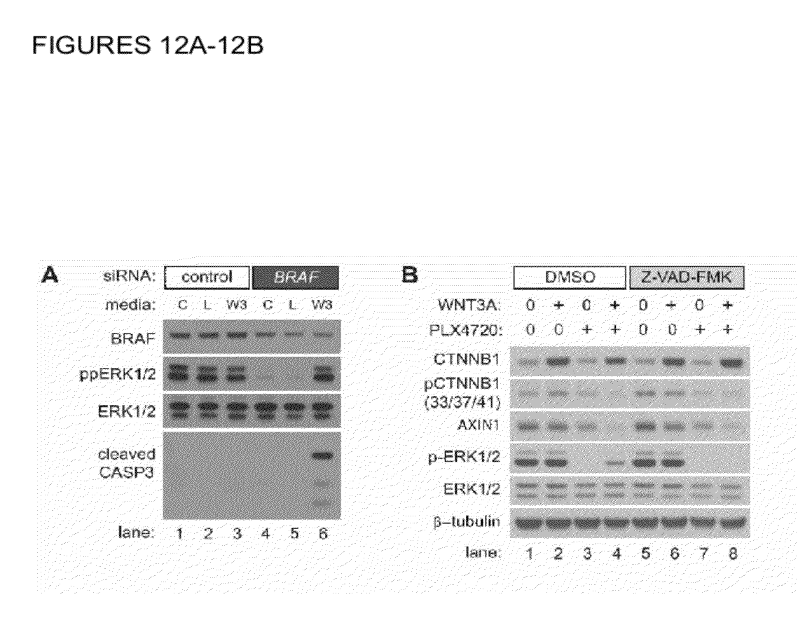

To establish that the effects of PLX4720 on apoptosis were specifically due to inhibition of BRAF^{V600E}, it was demonstrated that knockdown of BRAF by siRNA mimics the ability of PLX4720 to enhance the cleavage of caspase-3 in the presence of WNT3A (FIGS. 12A-12B). Together, these data demonstrate that simultaneous activation of Wnt/β-catenin signaling and inhibition of BRAF^{V600E} functionally cooperates to induce caspase-mediated apoptosis of melanoma cells.

In order to understand how activation of Wnt/β-catenin signaling cooperates with inhibition of BRAF^{V600E} to induce apoptosis in melanoma cells, levels of the Bcl-2 homology domain 3 only (BH3-only) proteins BAD and Bim (BCL2L11), both of which have been specifically implicated in melanoma as important regulators of apoptosis downstream of BRAF/MEK activation (27-32) were measured. Inhibition of BRAF or MEK leads to decreased phosphorylation of Ser75 on BAD, allowing BAD to neutralize its anti-apoptotic binding partners BCL-2, BCL-XL, and BCL-W (33). As expected, treatment of melanoma cells with PLX4720 led to decreased Ser75 phosphorylation (pBAD; FIG. 3D, lanes 1 versus 3 and lanes 5 versus 7). Co-treatment with WNT3A leads to an apparent rescue of the PLX4720-mediated decrease in Ser75 phosphorylation (FIG. 3D, lane 4 versus lane 3). To show that this rescue was the consequence of apoptosis-dependent reactivation of ppERK1/2 rather than a direct effect of WNT3A, cells were concomitantly treated with Z-VAD-FMK, which blocked ppERK1/2 reactivation and the apparent increase in Ser75 phosphorylation seen with PLX4720 plus WNT3A (FIG. 3D, lane 8 versus lane 4). These data demonstrate that WNT3A has no effect on phosphorylation of Ser75 on BAD.

The effects of activating Wnt/β-catenin signaling and inhibiting BRAF^{V600E} on Bim expression were next explored. As expected from previous reports, treatment with PLX4720 leads to decreased phosphorylation of the largest Bim isoform, Bim_{EL}, evidenced by an apparent shift in relative electrophoretic mobility (FIG. 3D, lane 3 versus lane 1 and lane 7 versus lane 5). Furthermore, the combination of WNT3A and PLX4720 leads to a marked increase in Bim_{L} and Bim_{S} (FIG. 3D, lane 4 versus 3 and lane 8 versus 7), two splice isoforms of Bim_{EL} involved in initiating apoptosis with targeted BRAF/MEK inhibition in melanoma cells (29, 31, 34). This increase in Bim_{L} and Bim_{S} is not blocked by Z-VAD-FMK (FIG. 3D, lane 8 versus lane 4), consistent with its role as an upstream activator of caspase-3 during apoptosis. These results implicate the regulation of Bim_{L} and Bim_{S} expression or splicing/processing as a potential site of cross-talk in the regulation of apoptosis by Wnt/β-catenin and BRAF/MAPK signaling. Together these data reveal that WNT3A increases the effectiveness of a BRAF^{V600E} inhibitor in promoting apoptosis (FIG. 3), which likely explains how WNT3A increases the effectiveness of a BRAF^{V600E} inhibitor in reducing tumor growth, spheroid growth and cell viability (FIG. 2A). The apoptosis seen with WNT3A and PLX4720 correlates with synergistic increases in Bim_{L} and Bim_{S} levels, without any observed Wnt-dependent changes in BAD phosphorylation.

Endogenous β-Catenin is Required for PLX4720 to Induce Apoptosis

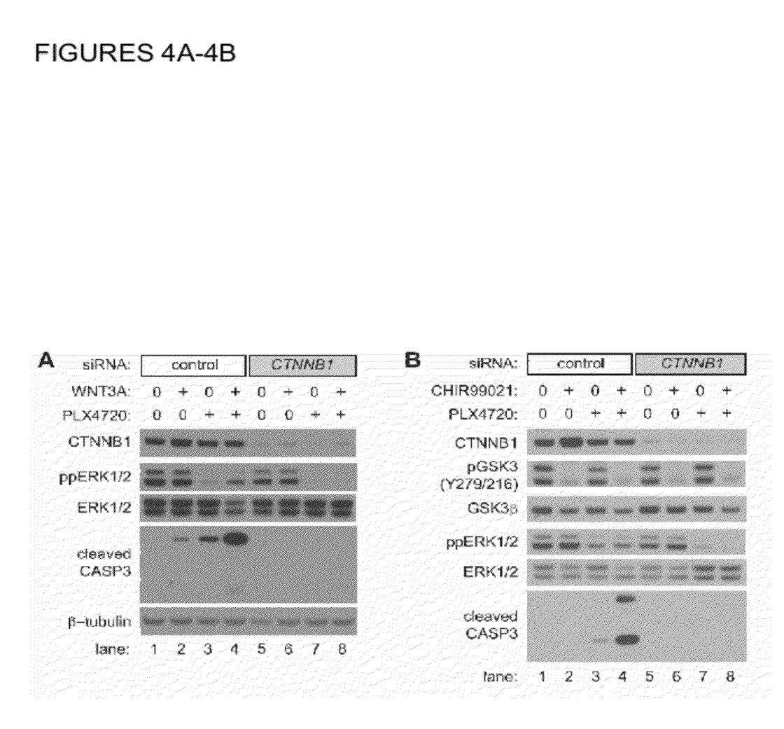

Whether the BRAF^{V600E} inhibitor PLX4720 requires an intact Wnt/β-catenin pathway for its ability to induce apoptosis was investigated. Strikingly, β-catenin siRNA completely prevents apoptosis of A375 cells treated with PLX4720 (FIG. 4A, lane 3 versus lane 7; FIG. 14E). This dependence on endogenous β-catenin for PLX4720 to elevate apoptosis was not overcome by exogenous WNT3A (FIG. 4A, lane 4 versus lane 8). We then activated β-catenin signaling downstream of the Wnt/receptor complex by treating cells with the small molecule GSK3B inhibitor CHIR99021. Like WNT3A, CHIR99021 enhanced apoptosis in combination with PLX4720, and this apoptosis was completely inhibited upon siRNA knockdown of β-catenin (FIG. 4B, lane 4 versus lane 8). Knockdown of AXIN1/2 by siRNA enhances Wnt/β-catenin signaling (FIG. 8A) and also enhances the apoptosis seen with the addition of PLX4720 (FIG. 14E). These data support the unexpected conclusion that apoptosis mediated by targeted BRAF inhibition is dependent upon β-catenin, the primary downstream effector of Wnt/β-catenin signaling.

PLX4720-Mediated Enhancement of Wnt/β-Catenin Signaling Predicts Apoptosis Among Melanoma Cell Lines.

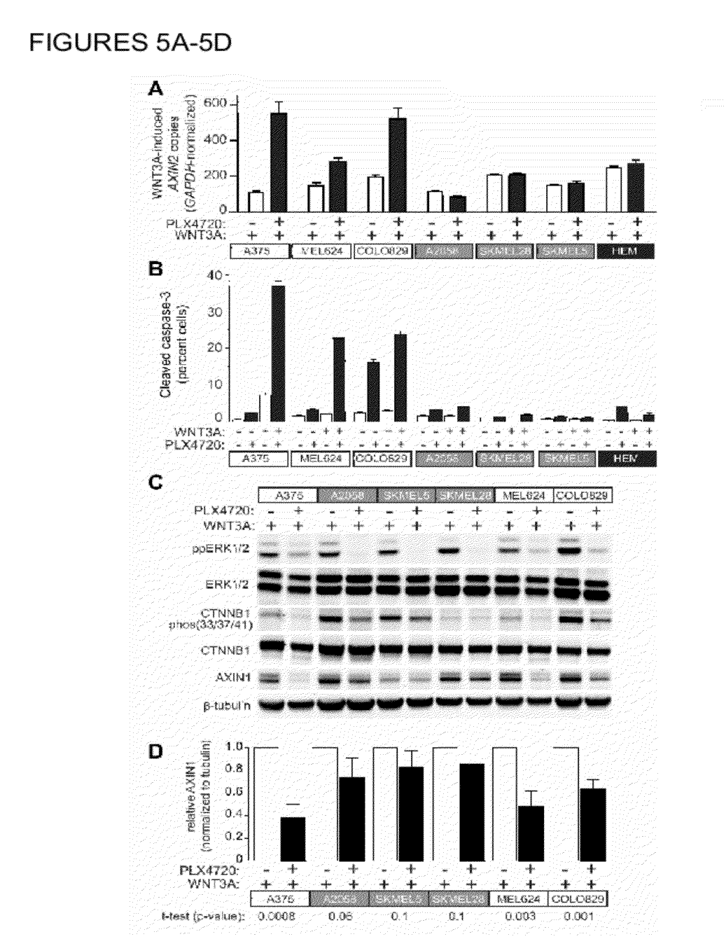

A significant number of patients with tumors harboring activating BRAF mutations do not exhibit a clinical response to targeted BRAF inhibitors (7), suggesting the involvement of as yet unidentified proteins and/or pathways that determine cellular susceptibility to therapy. The interaction between Wnt/β-catenin and BRAF/MAPK signaling in multiple melanoma cell lines that harbor the BRAF^{V600E} mutation was examined in order to uncover new insights into the heterogeneity of the response to targeted BRAF inhibitors. In A375, Mel624 and COLO829 cells, treatment with WNT3A increased the levels of transcripts encoding AXIN2, a known target gene of β-catenin signaling (11, 35), and co-treatment with PLX4720 led to further increases in levels of AXIN2 transcripts (FIG. 5A). In contrast, treatment with PLX4720 did not elevate the WNT3A-mediated increases in AXIN2 transcripts in SKMEL5, SKMEL28, and A2058 cells (FIG. 5A), despite the fact that these cells also harbor the BRAF^{V600E} mutation (Table 2). Interestingly, cell lines that display synergistic activation of Wnt/β-catenin signaling with WNT3A and PLX4720 also exhibit increased susceptibility to apoptosis as measured by cleaved caspase-3 (FIG. 5B). These data are consistent with a model in which Wnt/β-catenin signaling is a major determinant of the apoptotic response to targeted BRAF inhibition (FIGS. 4A and 4B). Of note, significant upregulation of AXIN2 transcript is seen in COLO829 even in the absence of exogenous Wnt3A, suggesting that this cell line may have functionally significant levels of endogenous Wnt/β-catenin signaling. The discrepancy in response among these cell lines cannot be accounted for by the allelic status of the BRAF^{V600E} mutation (Table 2). These experiments provide some of the first data addressing potential mechanisms that might explain the observed variations in clinical response to targeted inhibitors of BRAF among tumors carrying the BRAF^{V600E} mutation (5-7).

Inhibition of BRAF Signaling Leads to Wnt-Dependent Decreases in AXIN1

The correlation between Wnt/β-catenin signaling and apoptotic response (FIGS. 5A and 5B) led to further investigation of the underlying mechanism. Interestingly, in the three melanoma cell lines that are sensitive to apoptosis mediated by the combination of WNT3A plus PLX4720 (A375, Mel624, and COLO829), steady-state levels of the β-catenin antagonist AXIN1 were markedly reduced with WNT3A plus PLX4720 when compared to WNT3A treatment alone (FIG. 5C). This reduction was statistically significant when the protein signal on immunoblots from three separate experiments was quantified and normalized to levels of β-tubulin (FIG. 5D). By contrast, in the melanoma cell lines that are resistant to apoptosis with WNT3A plus PLX4720 (SKMEL5, SKMEL28 and A2058) steady-state AXIN1 levels did not significantly decrease when comparing treatment with WNT3A alone to WNT3A plus PLX4720 (FIGS. 5C and 5D). Together, these data demonstrate a direct correlation between the ability of PLX4720 to decrease steady-state levels of AXIN1 in the presence of WNT3A and the susceptibility of melanoma cells to apoptosis.

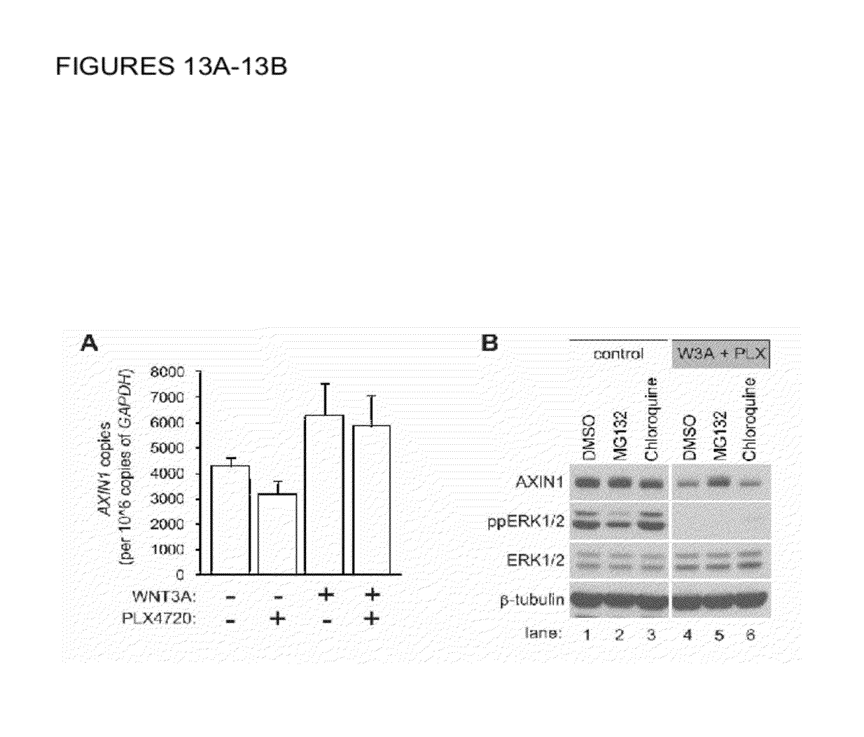

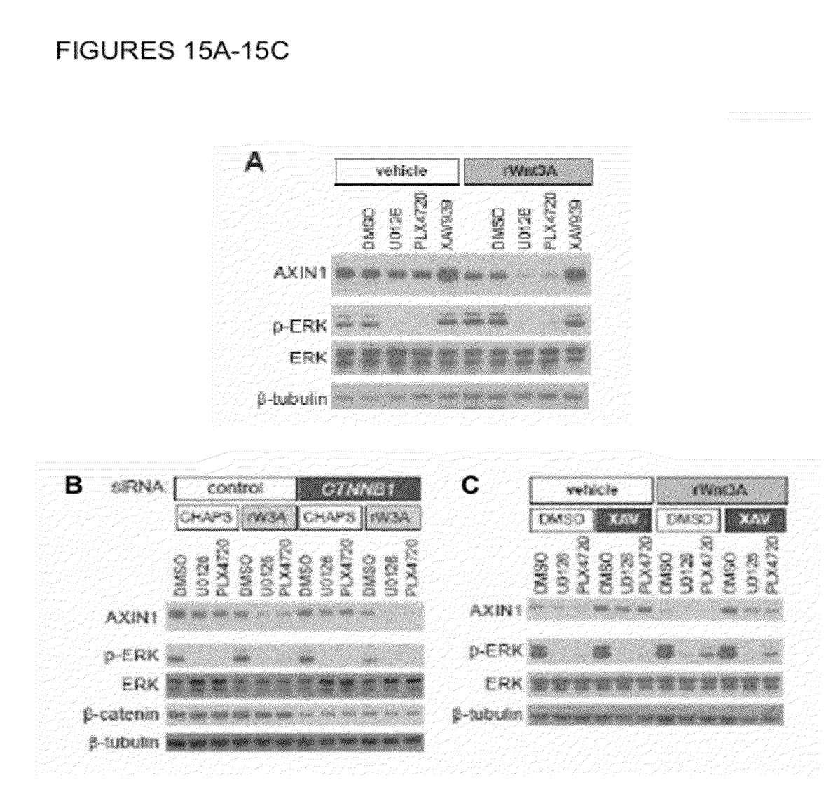

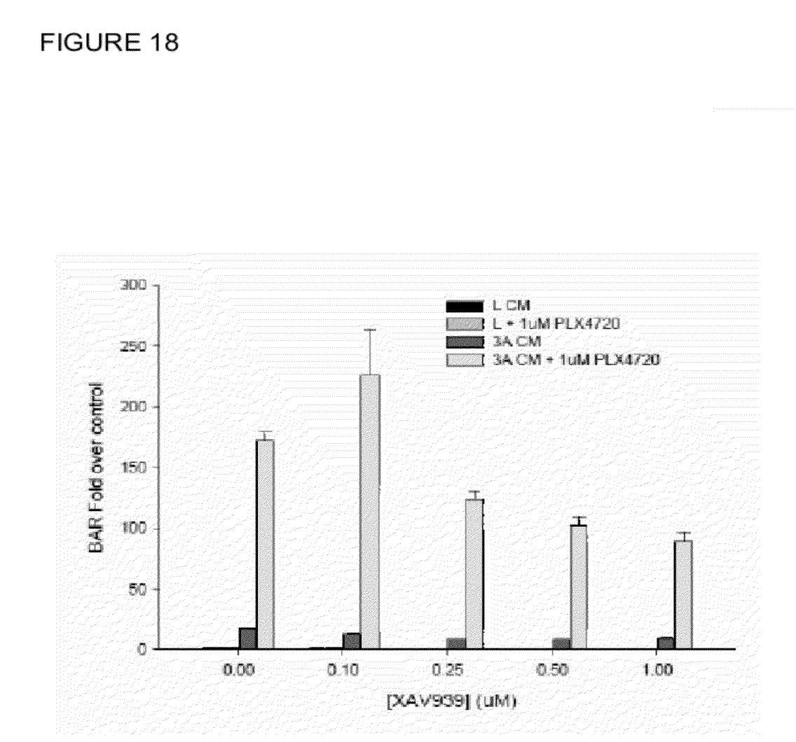

Melanoma cells were treated with either vehicle or purified recombinant Wnt3A (rWnt3A) in the presence of DMSO, U0126, and PLX4720 (FIG. 13A, FIG. 15A). In the absence of rWnt3A, steady-state levels of AXIN1 are slightly decreased upon treatment with U0126 and PLX4720 (FIG. 15A, left panel). In the presence of rWnt3A, AXIN1 steady-state levels are markedly diminished (FIG. 15A, right panel). In the absence and presence of rWnt3A, the small molecule XAV939 increases steady-state levels of AXIN1 (FIG. 15A), as predicted by its known activity as a tankyrase inhibitor. In support of the hypothesis that AXIN1 levels are regulated by MAPK signaling at the post-translational level, no transcriptional regulation of AXIN1 gene expression is observed upon treatment by either Wnt3A or PLX4720 (FIG. 13A). Furthermore, decreased steady-state levels of AXIN1 upon MAPK inhibition by either U0126 or PLX4720 are seen both in the absence and presence of rWnt3A even upon RNAi-mediated knockdown of β-catenin (CTNNB1), indicating that downstream Wnt-dependent transcriptional responses are not involved (FIG. 15B). Even in the presence of U0126 or PLX4720, XAV939 can elevate AX1N1 protein levels to above baseline levels (FIG. 15C) but is unable to completely reverse the enhancement of Wnt/β-catenin signaling by MAPK inhibition (FIG. 18). Whether WNT3A plus PLX4720 promotes proteasomal or lysosomal degradation of AXIN1 was next investigated. Treatment of A375 cells with MG132, but not chloroquine, increased the levels of AXIN1 following treatment with WNT3A and PLX4720 (FIG. 13B, lane 5 versus lane 4 and lane 6 versus lane 4). Together, these data reveal that BRAF^{V600E} inhibition regulates AXIN1 levels in the presence of WNT3A through a proteasome-dependent mechanism.

The effects of MAPK inhibition on AXIN1 levels across six separate established melanoma lines harboring the BRAF^{V600E} mutation were examined, revealing inhibition of ERK1/2 phosphorylation upon treatment with either U0126 (data not shown) or PLX4720 (FIG. 5C). While the addition of Wnt3a reduces phosphorylation of β-catenin at Ser33/Ser37/Thr41 as expected in all cell lines (data not shown), phosphorylation is further inhibited by the addition of PLX4720 (FIG. 5C). Interestingly, in the presence of Wnt3A, decreased levels of AXIN1 are readily seen by immunoblotting in three of these cell lines: A375, Mel624 and COLO829 (FIG. 3E). In SKMEL5, SKMEL28 and A2058 cells, inhibition of BRAF/MAPK signaling by PLX4720 did not have marked effects on AXIN1 (FIG. 5C). Together, these findings suggest that steady-state levels of AXIN1 are maintained in a subset of BRAF^{V600E} melanoma cell lines by BRAF/MAPK signaling, and that this regulation is both independent of Wnt/β-catenin-dependent transcription and potentially dependent on cellular localization events related to ligand binding.

Loss of AXIN1 Precedes Apoptosis and can Confer Susceptibility to Apoptosis with BRAF Inhibition.

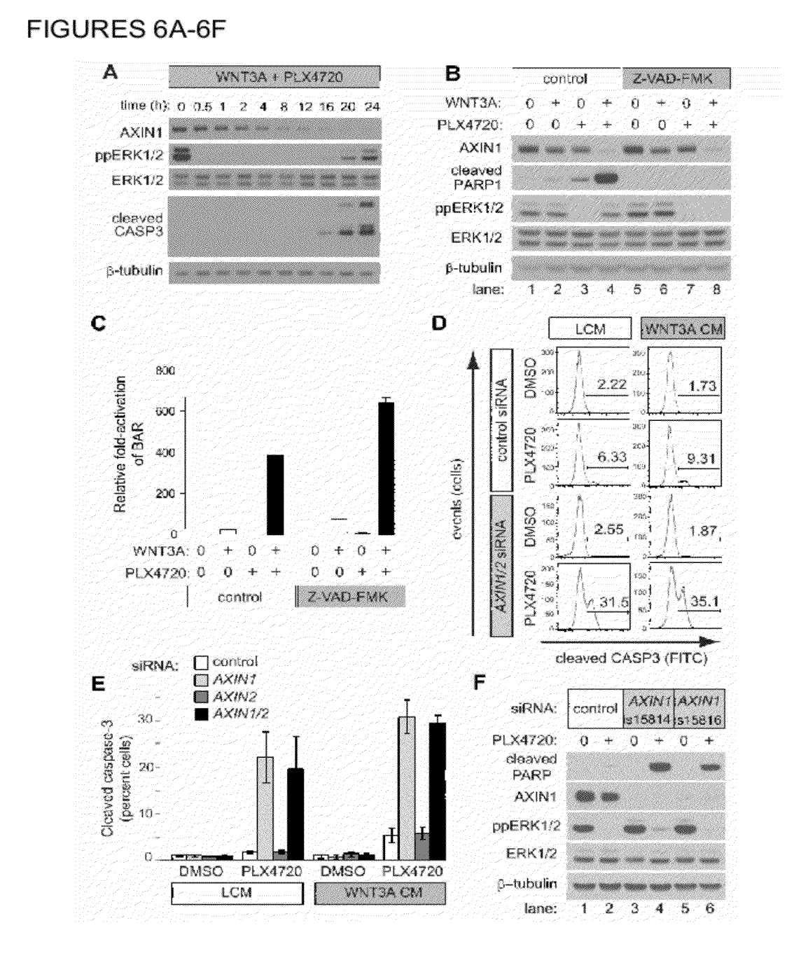

To determine if decreased AXIN1 protein levels sensitize melanoma cells to PLX4720-mediated apoptosis, the temporal coordination of ppERK relative to levels of AXIN1 and to the onset of apoptosis was investigated. A time course of A375 cells treated with WNT3A and PLX4720 followed by immunoblotting was performed. A rapid decrease in steady-state levels of AXIN1 occurred within 1-2 hours of initiating treatment, with almost no detectable AXIN1 remaining after 16-20 hours of treatment (FIG. 6A). This decrease in AXIN1 levels followed the rapid inhibition of ppERK, which occurred within 30 minutes. Apoptosis as measured by cleaved caspase-3 was first detected at 12-16 hours, and increased for the duration of the experiment (FIG. 6A). Furthermore, while the pan-caspase inhibitor Z-VAD-FMK was able to inhibit apoptosis in these cells (FIG. 6B) it did not affect the loss of steady-state AXIN1, indicating that the decrease in AXIN1 is not a downstream consequence of caspase-3 activation. These data suggest that decreases in AXIN1 levels precede the onset of apoptosis, raising the question of whether AXIN1 is functionally involved in mediating apoptosis. Furthermore, the inhibition of apoptosis by Z-VAD-FMK augments Wnt/β-catenin activation as measured by BAR (FIG. 6C), indicating that the enhanced Wnt/β-catenin signaling observed in cell lines that exhibit significant apoptosis with WNT3A plus PLX4720 (FIGS. 5A and 5B) is upstream of caspase-3 activation.

Interestingly, the onset of apoptosis coincides with detection of phospho-ERK, raising the possibility that cells undergoing apoptosis may be activating MAPK signaling downstream of BRAF. In support of this hypothesis, phospho-ERK in the presence of Wnt3A and PLX4720 is only detected in the three cell lines (A375, Mel624 and COLO829) that exhibit apoptosis (FIG. 5C). Phospho-ERK is also seen in other conditions where apoptosis is observed, such as in the presence of Wnt3A and BRAF siRNA (FIG. 12A), and not seen under conditions in which apoptosis is blocked, such as with Wnt3a and PLX4720 in the presence of β-catenin knockdown (FIG. 16C).

Given that decreases in AXIN1 levels (FIGS. 5C, 5D and 6A) precede apoptosis and seem to predict both susceptibility to Wnt-driven apoptosis (FIG. 5B) and to enhancement of Wnt/β-catenin signaling by BRAF inhibition (FIG. 5A), it was hypothesized that reducing levels of AXIN1 in melanoma cell lines that are more resistant to apoptosis would render them newly susceptible to apoptosis in the presence of PLX4720. Since functional in vivo redundancy of AXIN1 and AXIN2 has been previously described (36), initial studies were performed by knocking down both isoforms. Indeed, siRNA-mediated knockdown of AXIN1/2 in SKMEL28 (FIG. 6D), A2058 (Table 3; FIGS. 17A-B) and SKMEL5 (Table 3) cells led to increased apoptosis with PLX4720 as measured by cleaved caspase-3. When AXIN1 and AXIN2 were targeted individually by siRNA, enhancement of apoptosis was seen only with knockdown of AXIN1 and not AXIN2 (FIG. 6E). The ability of AXIN1, but not AXIN2, knockdown to confer susceptibility to apoptosis with PLX4720 was confirmed using two independent and validated siRNAs for each target (FIG. 6F). These results strongly argue that AXIN1 levels play an important and previously unsuspected role in the regulation of apoptosis by ERK signaling in melanoma cells.

### Example 2

### Discussion

While the unprecedented response rates in early clinical trials with PLX4032/vemurafenib and GSK2118436 are extremely promising, there are still significant obstacles to achieving long-term disease control with this approach. For example, up to half of patients with BRAF^{V600E} tumors exhibit no clinical response with targeted BRAF inhibition (5-7). The discovery described herein that regulation of AXIN1 levels can determine apoptotic response to inhibition of BRAF^{V600E} provides the first biochemical demonstration that cellular signaling determinants downstream of BRAF can be correlated with the variable response to PLX4720 seen across different BRAF^{V600E} cell lines. Furthermore, the findings described herein that targeting AXIN1 levels can confer susceptibility to apoptosis with BRAF inhibition in previously unresponsive cell lines indicates that these cell-specific differences can be identified and that the unresponsiveness to the drug therapy can be overcome.

Another ongoing clinical problem is the eventual development of resistant tumors and the progression of the disease even in patients who respond well to initial therapy (7). This indicates that the targeting of multiple regulatory pathways will likely be required to achieve a durable clinical result. While combination targeting of BRAF/MAPK signaling has been suggested with other pathways implicated in melanoma, such as the PI3K/AKT pathway (37,38), the results presented herein provide the first indication that an interaction between BRAF/MAPK signaling and Wnt/β-catenin signaling in melanoma has potential therapeutic implications for melanoma patients.

Therapeutically, the findings described herein indicate that activation of Wnt/β-catenin signaling can greatly improve the efficacy of treating melanoma patients with targeted BRAF inhibitors. Consistent with the observation described herein that β-catenin is required for the apoptosis seen with PLX4720, the transcriptional profiling of melanoma lines revealed that cell lines that are more resistant to growth inhibition by PLX4032 exhibit the apparent loss of genes related to active Wnt/β-catenin signaling while upregulating markers of neuronal precursors (39). Furthermore, cell lines susceptible to PLX4032 treatment exhibit a more melanocyte-like gene signature, similar to the effects previously reported by the inventors with Wnt/β-catenin activation in melanoma (11).

The notion of activating Wnt/β-catenin signaling seems counter-intuitive to its frequent role as an oncogenic pathway (17). However, the activation of this pathway in melanomas is considerably different from the well-described role of Wnt/β-catenin signaling in colorectal carcinoma, where constitutive pathway activation occurs largely through genetic mutations in adenomatous polyposis coli (APC). In fact, activating mutations in the Wnt/β-catenin pathway are rare in melanoma cell lines (17), suggesting that the observed presence of nuclear β-catenin in the majority of nevi and a significant percentage of melanomas represents activation of the pathway from ligand-driven signaling. From the viewpoint of both therapeutics and maintenance of homeostasis, a cell with ligand-driven Wnt/β-catenin signaling that can be dynamically regulated by cellular feedback mechanisms presents an entirely different context than a cell with near maximally-activated mutation-driven Wnt/β-catenin signaling, as seen in colorectal carcinoma. Without wishing to be bound by theory, this difference likely contributes to discrepancies in the reported consequences of Wnt/β-catenin activation in melanoma seen with models that activate the pathway with mutant β-catenin (40) compared to models that use WNT3A ligand (11), which may likely be a better representation of the context seen in patient melanoma tumors. The previous identification and validation of patient-experienced small molecule synergistic activators of Wnt/β-catenin signaling (41) provides options for combination therapies that can enhance the clinical effects of targeted BRAF inhibition through the augmentation of pre-existing Wnt/β-catenin signaling.

Depending on cellular context, Wnt/β-catenin signaling has been shown to both prevent or facilitate programmed cell death (22). For example, early in the developing hindbrain, geographical activation of Wnt/β-catenin signaling mediates the selective apoptosis of pre-migratory neural crest cells (42), while later on during development Wnt/β-catenin signaling is required for the proliferation and differentiation of the neural tube (43). With regards to melanoma, previous studies using various cultured cell models have reported increased apoptosis with inhibition of Wnt/β-catenin signaling (40, 44-47), which was not observed in the experiments presented herein. The synergistic enhancement of Bim_{S} levels by Wnt/β-catenin activation and targeted BRAF inhibition (FIG. 3D) has not been previously reported, although pharmacological inhibition of GSK3β by means other than WNT3A enhanced Bim expression and cell death in glioma (48).

The observed effects of BRAF inhibition on steady-state AXIN1 levels and GSK3β phosphorylation and activation (FIG. 1C) suggest a mechanism that involves the regulation of β-catenin phosphorylation and potentially its subsequent ubiquitination and degradation. However, the lack of further increase in β-catenin levels with PLX4720 treatment suggests that the resultant enhancement of Wnt/β-catenin signaling evidenced by the reporter assay (BAR) and the increased expression of the endogenous target gene activation (AXIN2) does not require additional β-catenin accumulation (50-52). The recent report that loss of phosphorylation of β-catenin at Thr41 is sufficient to enhance Wnt/β-catenin signaling in melanoma cells independent of detected increases in nuclear β-catenin is entirely consistent with the data presented herein (25). While decreased phosphorylation of β-catenin with PLX4720 is seen across all cell lines tested (FIG. 5C), the observed increase in AXIN2 levels with PLX4720 in only half of these lines (FIG. 5A) suggests that decreased β-catenin phosphorylation alone is not sufficient to enhance Wnt/β-catenin signaling with targeted BRAF inhibition.

### Example 3

### Materials and Methods

Reagents. Detailed information on the β-catenin activated reporter (BAR) has been previously described (23). Briefly, the β-catenin activated reporter (pBAR) is a lentiviral plasmid that contains 12 TCF/LEF binding sites (5′-AGATCAAAGG-3′) each separated by distinct 5 base-pair linkers upstream of a minimal promoter and the firefly luciferase open reading frame. The reporter also contains a separate PGK promoter that constitutively drives the expression of a puromycin resistance gene for mammalian cell selection. Transient transfection of siRNA was performed with RNAiMAX, as directed by the manufacturer (13778-075, Invitrogen). siRNA sequences used are listed in Table 1. Protease (#11873580001) and phosphatase (#04906845001) inhibitor tablets were purchased from Roche (Indianapolis, Ind.). Con A Sepharose was purchased from GE Healthcare (Uppsala, Sweden #17-0440-03). U0126 was purchased from LC labs (Woburn, Mass. cat# U-6770). AZD6244 was purchased from Selleck Chemicals (Houston, Tex. cat# S1008). PLX4720 was purchased from Symansis (Australia cat# SY-PLX4720). CHIR99021 was purchased from Axon MedChem (Gronigen, Netherlands catalog #Axon1386). Z-VAD-FMK was purchased from R&D systems (Minneapolis, Minn. cat# FMKSP01). Anti-ERK (p42/44) (#9102), anti-phopho-ERK (p42/44) (#9101S), anti-phospho-β-catenin S33/37/T41 (#9561S), anti-BRAF (#9434), anti-cleaved CASP3 (#9661S), anti-cleaved PARP1 (#9541), anti-Bim (#2933), anti-Bad (#9239), anti-phospho-Bad (#5284), and anti-cleaved CASP3 Alexafluor 488 conjugate (#9669) antibodies were purchased from Cell Signaling (Cell Signaling, Beverly Mass.). Anti-β-tubulin (T7816) and anti-β-catenin (C2206) antibodies were purchased from Sigma Aldrich (Sigma Aldrich St. Louis, Mo.). Anti-phospho-GSK3 Y279/216 (05-413) was purchased from Upstate Biotechnology (Waltham, Mass.). anti-AXIN1 (AF3287) antibody was purchased from R&D Systems (Minneapolis, Minn.). In Situ Cell Death Detection kit (cat#12156 792 910) was purchased from Roche (Indianapolis, Ind.).

Cell Lines. The human melanoma cell lines A375, A2058 and Me1624 were obtained from Cassian Yee (Fred Hutchinson Cancer Research Institute; Seattle, Wash.). The human melanoma cell lines COLO-829, SKMEL28, SKMEL5 were purchased from ATCC (Manassas, Va.). Human Epidermal Melanocytes, adult, lightly pigmented donor, (HEMa-LP) (C0245C) were purchased from Invitrogen (Carlsbad, Calif.). Stable BAR cell lines were generated as previously described (23). BAR luciferase cell lines were also infected with a lentivirus carrying Renilla luciferase driven by a constitutive EFlalpha promoter.

Cell Culture. The human melanoma lines A375, A2058 were cultured in DMEM supplemented with 5% FBS and 1% antibiotic. The human melanoma lines SK-MEL-5 and SK-MEL-28 were grown in EMEM supplemented with 10% FBS and 1% antibiotic. The human melanoma lines COLO-829 and MEL-624 were grown in RPMI supplemented with 10% FBS and 1% antibiotic HEMa-LP cells were cultured in medium 254 supplemented with 1% HMGS and 1% antibiotic (Invitrogen Carlsbad, Calif.). Synthetic siRNAs were transfected into cultured cells at a final concentration of 20 nM using RNAiMAX (Invitrogen; Grand Island, N.Y.).

High Throughput Screening. A library of siRNAs targeting primarily the human kinome was screened in A375 melanoma cells stably expressing the β-catenin activated reporter (BAR). The kinome siRNA library was purchased from Sigma Aldrich (Sigma Aldrich St. Louis, Mo.) and resuspended in RNase free water. The library consists of a pool of three independent non-overlapping siRNAs for each mRNA target. siRNA pools were screened in quadruplicate at 9.5 nM, 1.9 nM, 0.38 nM, and 0.08 nM final concentration. Cell viability was assessed by adding resazurine (Sigma Aldrich St. Louis, Mo.) at a final concentration of 1.25 ug/ml (PBS vehicle) and measuring fluorescence intensity (Ex=530 nM Em=580 nM) on an Envision multilabel plate reader (Perkin Elmer Waltham, Mass.). Luciferase activity was assessed by adding 5 uL/well SteadyGlo (Promega Madison, Wis.) and measuring total luminescence on an Envision multilabel plate reader (Perkin Elmer Waltham, Mass.) The screen workflow was as follows: On day 1, 1.5 uL of the appropriate concentration of siRNA was added to 28.5 uL of Optimem (Invitrogen, Carlsbad, Calif.) containing 3.125 uL/mL RNAiMAX (Invitrogen, Carlsbad, Calif.). 5 uL of this mix was transferred to a 384 well plate containing 15 uL of growth media (DMEM/5% FBS/1% PenStrep). 20 uL of cells at 75 cells/uL was added to each well for a final cell number of 1500 cells/well. On day 3, 10 uL of WNT3A conditioned media diluted 1:12.8 with growth media was added for a final dilution of 1:64. On Day 4, 10 uL of 6× resazurine was added to each well, incubated at 37° C. for three hours, and fluorescence intensity was measured. Immediately following, 5 uL of SteadyGlo was added, incubated at room temperature for 10 minutes and total luminescence was measured. Data are represented as BAR reporter activity (Luminescence)/cell viability (Resazurine fluorescence intensity).

Low throughput BAR reporter assays. Cells were plated in 96-well plates. 24 hours following plating, control or WNT3A stimulus and/or chemicals were added and luciferase activity was measured 24 hours later with the a dual luciferase reporter assay kit (Promega; Madison, Wis.) and an Envision multi-label plate reader (Perkin Elmer, Waltham, Mass.) per manufactures suggestions. For BAR assays involving siRNAs, siRNAs were transfected 48 hours before stimulus and/or chemical addition.

Cytosolic and Nuclear β-catenin Fractionation. Cells were plated in 100 mm dishes. 24 hours following plating, cells were treated with the indicated conditions for 24 hours. Cells were gently rinsed with PBS and harvested by scraping in 500 uL of hypotonic lysis buffer (50 mM HEPES pH 8.0, 1 mM EDTA, 1 mM DTT) containing protease and phosphatase inhibitors. Cells were swelled on ice for 30 minutes and then passed through a 27 gauge needle ten times and checked for complete lysis with a microscope. Lysates were centrifuged at 10,000×g for 20 minutes and supernatant was collected as the cytosolic fraction. Pelleted membranes were washed 5 times with hypotonic lysis buffer and then solubilized with solubilization buffer (50 mM Tris pH 8.0, 150 mM NaCl, 0.1% SDS, 0.5% sodium deoxycholate, 1% Triton X-100) containing protease and phosphatase inhibitors. After a 30 minute incubation on ice, lysates were centrifuged at 16,000×g for 20 minutes. The protein concentration of the cleared supernatant was determined by BCA analysis and an equal amount of protein and volume was then incubated with pre-washed Con A sepharose beads overnight at 4 degree C. Supernatant was collected as the nuclear fraction.

RNA purification and qRT-PCR analysis. RNA was purified using the RNeasy kit following the manufacturer's protocol (Qiagen; Maryland, Md.). cDNA was synthesized using RevertAid™ M-MuLV Reverse Transcriptase (Fermentas; Ontario, CAN). Light Cycler FastStart DNA Master SYBR Green1 (Roche; Mannheim, Germany) was used for real-time PCR as previously described (53). Quantitative PCR results presented in the manuscript are averages of a minimum of three biologic replicates.

Isobologram Analysis of Cell Viability. A375 melanoma cells were seeded in 96-well plates at a concentration of 8,000 cells/well in 100 μl of growth media. 24 hours after plating, cells were treated with all combinations of 2-fold dilutions of WNT3A CM ranging from 20% to 0% and 2-fold dilutions of PLX4720 ranging from 5 μM-0 μM for 48 hours. 10 uL of CellTiter-Glo (Promega Madison, Wis.) was added to each well and total luminescence was measured on an Envision multilabel plate reader (Perkin Elmer Waltham, Mass.). Each condition within an experiment was assayed in triplicate wells and three independent experiments were performed.

Flow cytometry for Active Caspase-3. Cells were seeded in a 6-well dish at a density to achieve 90-100% confluence at harvest. 24 hours after seeding, cells were treated with the indicated conditions for the indicated amount of time. At the time of collection, supernatants were collected and pooled with trypsinized cells. Cells were fixed with 4% paraformaldehyde and permeabilized according to vendor's protocol for Cleaved Caspase-3 (Asp175) Antibody (AlexaFluor 488 Conjugate) (catalog #9669) (Cell Signaling, Beverly Mass.). The antibody was used at a final dilution of 1:100. Flow was performed on a BD FACSCanto H, and data analysed with FlowJo 8.8.6 (Tree Star) software. Experiments were performed with biological triplicates and data are representative of at least three independent experiments.

For experiments involving siRNAs, cells were reverse transfected with 20 nM siRNA in 6-well dishes in triplicate with RNAiMax according to manufacturer's protocol. 48 hours following transfection, cells were treated with the indicated conditions for 24 hours and then harvested for analysis. Cells were harvested, stained, and analyzed as described above.

TUNEL. Glass coverslips were coated with poly-L-lysine in a 24-well dish, rinsed with PBS, and dried. Cells were seeded at a density to achieve 90-100% at harvest. 24 hours after seeding, cells were treated with the indicated conditions and incubated for 24 hours. TUNEL staining was performed according to vendor's protocol (cat#12 156 792 910) (Roche Indianapolis, Ind.). Briefly, media was gently aspirated and cells were fixed in 4% paraformaldehyde for 1 hour at room temperature. Cells were gently rinsed twice with PBS and permeabilized with 0.1% Triton X-100 in 0.1% sodium citrate for 2 minutes on ice. Cells were rinsed twice with PBS, and 40 uL of TUNEL reaction mixture was added directly on top of the slide and incubated for 1 hour at 37° C. in a humidified incubator. Slips were rinsed 3 times and mounted on superfrost plus glass slides (cat#48311-703 VWR West Chester, Pa.) with Prolong Gold anti-fade mounting media containing DAPI (cat# P36931 Invitrogen; Grand Island, N.Y.). Images were obtained on a Nikon TiE inverted widefield high-resolution microscope (Nikon Melville, N.Y.).

Spheroid Assay. A375 cells were used for the spheroid assays. Spheroids were formed and implanted in collagen as previously described (54). Spheroids were treated with indicated conditions 30 minutes after collagen polymerization. Images were obtained on a Nikon TiE inverted widefield high-resolution microscope (Nikon Melville, N.Y.). For comparison of growth effects such as shown in FIG. 2, spheroids were imaged at 72 hours after spheroid implantation. For live-dead imaging assays such as shown in FIG. 3, imaging was performed at 24 hours after spheroid implantation.

Xenograft assays. NSG (NOD/SCID/IL2r-gamma (null)) mice were injected with 5×10⁵ A375 cells stably expressing GFP or 5×10⁵ A375 cells stably expressing WNT3A-IRES-GFP. Tumors were allowed to establish to approximately 100 mm³, after which mice where tumor size-matched and allocated to five per treatment group (vehicle or PLX4720). WNT3A-IRES-GFP tumors grew slower and therefore the first day of treatment was day 14 while GFP expressing tumors were first treated on day 9. Treatment was by oral gavage once daily with 5% DMSO in 1% carboxymethyl cellulose or 50 mg/kg PLX4720 in 1% carboxymethyl cellulose (604 mM PLX4720 in DMSO was diluted 1:20 in 1% carboxymethylcellulose). Tumor size was determined by caliper measurements of tumor length and width every 3 to 4 days. Tumor volume was then calculated using the following formula: volume=(width)²×length/2. Tumors were harvested 2 hours after the last dose and fixed in neutral-buffered formalin overnight at room temperature.

Mitotic index. Hematoxylin- and eosin-stained tumor sections were scored for mitotic activity by a board-certified pathologist who was blinded to the treatment conditions. For each treatment condition, five tumors were evaluated and a range of 26-60 high-powered fields (hpf's) per individual tumor were scored (average of 44 hpf's per tumor). Areas with fixation artifact were excluded a priori from the final analysis, accounting for differences in the number of hpf's per individual tumor. Analysis was performed using a one-way ANOVA followed by a post-test for linear trend.

Statistical analysis. Standard statistical analysis was performed using GraphPad Prizm (GraphPad Inc., LaJolla Calif.) version 5.01. Dose-effect analyses, including combination indices, dose reduction indices and median-effect analysis for FIGS. 9A-9C and FIGS. 11A-11C were performed using the method of Chou and Talay (55) via the CalcuSyn software suite (Biosoft, Cambridge UK), version 2.1.

### Example 4

### References

1. Davies, H., et al., Mutations of the BRAF gene in human cancer. Nature, 2002.417(6892): p. 949-54.
2. Tsai, J., et al., Discovery of a selective inhibitor of oncogenic B-Raf kinase with potent antimelanoma activity. Proc Natl Acad Sci USA, 2008. 105(8): p. 3041-6.
3. Bollag, G., et al., Clinical efficacy of a RAF inhibitor needs broad target blockade in BRAF-mutant melanoma. Nature, 2010. 467(7315): p. 596-9.
4. Lee, J. T., et al., PLX4032, a potent inhibitor of the B-Raf V600E oncogene, selectively inhibits V600E-positive melanomas. Pigment Cell Melanoma Res, 2010. 23(6): p. 820-7.
5. Kefford, R. F., et al., Phase I/II study of GSK2118436, a selective inhibitor of oncogenic mutant BRAF kinase, in patients with metastatic melanoma and other solid tumors. J Clin Oncol, 2010. 28(15s): p. abstract 8503.
6. Chapman, P. B., et al., Improved Survival with Vemurafenib in Melanoma with BRAF V600E Mutation. New England Journal of Medicine, 2011. online advanced publication(0).
7. Flaherty, K., et al., Inhibition of mutated, activated BRAF in metastatic melanoma. N Engl J Med, 2010. 363(9): p. 809-819.
8. Chien, A. J., W. H. Conrad, and R. T. Moon, A Wnt survival guide: from flies to human disease. J Invest Dermatol, 2009. 129(7): p. 1614-27.
9. Delmas, V., et al., β-catenin induces immortalization of melanocytes by suppressing p16INK4a expression and cooperates with N-Ras in melanoma development. Genes Dev, 2007. 21(22): p. 2923-35.
10. Bachmann, I. M., et al., Importance of P-cadherin, β-catenin, and Wnt5a/frizzled for progression of melanocytic tumors and prognosis in cutaneous melanoma. Clin Cancer Res, 2005. 11(24 Pt 1): p. 8606-14.
11. Chien, A. J., et al., Activated Wnt/β-catenin signaling in melanoma is associated with decreased proliferation in patient tumors and a murine melanoma model. Proc Natl Acad Sci USA, 2009. 106(4): p. 1193-8.
12. Gould Rothberg, B. E., et al., Melanoma prognostic model using tissue microarrays and genetic algorithms. J Clin Oncol, 2009. 27(34): p. 5772-80.
13. Kageshita, T., et al., Loss of β-catenin expression associated with disease progression in malignant melanoma. Br J Dermatol, 2001. 145(2): p. 210-6.
14. Maelandsmo, G. M., et al., Reduced β-catenin expression in the cytoplasm of advanced-stage superficial spreading malignant melanoma. Clin Cancer Res, 2003. 9(9): p. 3383-8.
15. Omholt, K., et al., Cytoplasmic and nuclear accumulation of β-catenin is rarely caused by CTNNB1 exon 3 mutations in cutaneous malignant melanoma. Int J Cancer, 2001. 92(6): p. 839-42.
16. Rimm, D. L., et al., Frequent nuclear/cytoplasmic localization of β-catenin without exon 3 mutations in malignant melanoma. Am J Pathol, 1999. 154(2): p. 325-9.
17. Lucero, O. M., et al., A re-evaluation of the “oncogenic” nature of Wnt/β-catenin signaling in melanoma and other cancers. Curr Oncol Rep, 2010. 12(5): p. 314-8.
18. Chen, Z., et al., MAP kinases. Chem Rev, 2001. 101(8): p. 2449-76.
19. Yoon, S, and R. Seger, The extracellular signal-regulated kinase: multiple substrates regulate diverse cellular functions. Growth Factors, 2006. 24(1): p. 21-44.
20. Smalley, K. S., A pivotal role for ERK in the oncogenic behaviour of malignant melanoma? Int J Cancer, 2003. 104(5): p. 527-32.
21. Bikkavilli, R. K. and C. C. Malbon, Mitogen-activated protein kinases and Wnt/β-catenin signaling: Molecular conversations among signaling pathways. Commun Integr Biol, 2009. 2(1): p. 46-9.
22. Ji, H., et al., EGF-induced ERK activation promotes CK2-mediated disassociation of α-Catenin from β-Catenin and transactivation of beta-Catenin. Mol Cell, 2009. 36(4): p. 547-59.
23. Biechele, T. L. and R. T. Moon, Assaying β-catenin/TCF transcription with β-catenin/TCF transcription-based reporter constructs. Methods Mol Biol, 2008. 468: p. 99-110.
24. Hingorani, S. R., et al., Suppression of BRAF(V599E) in human melanoma abrogates transformation. Cancer Res, 2003. 63(17): p. 5198-202.
25. Kuphal, S. and A. K. Bosserhoff, Phosphorylation of β-catenin results in lack of β-catenin signaling in melanoma. Int J Oncol, 2011. 39(1): p. 235-43.
26. Dry, J. R., et al., Transcriptional pathway signatures predict MEK addiction and response to selumetinib (AZD6244). Cancer Res, 2010. 70(6): p. 2264-73.
27. Boisvert-Adamo, K. and A. E. Aplin, Mutant B-RAF mediates resistance to anoikis via Bad and Bim. Oncogene, 2008. 27(23): p. 3301-12.
28. Cartlidge, R. A., et al., Oncogenic BRAF(V600E) inhibits BIM expression to promote melanoma cell survival. Pigment Cell Melanoma Res, 2008. 21(5): p. 534-44.
29. Cragg, M. S., et al., Treatment of B-RAF mutant human tumor cells with a MEK inhibitor requires Bim and is enhanced by a BH3 mimetic. J Clin Invest, 2008. 118(11): p. 3651-9.
30. Gillings, A. S., et al., Apoptosis and autophagy: BIM as a mediator of tumour cell death in response to oncogene-targeted therapeutics. FEBS J, 2009. 276(21): p. 6050-62.
31. Jiang, C. C., et al., Apoptosis of human melanoma cells induced by inhibition of B-RAFV600E involves preferential splicing of bimS. Cell Death Dis, 2010. 1(9): p. e69.
32. Sheridan, C., G. Brumatti, and S. J. Martin, Oncogenic B-RafV600E inhibits apoptosis and promotes ERK-dependent inactivation of Bad and Bim. J Biol Chem, 2008. 283(32): p. 22128-35.
33. Danial, N. N., BAD: undertaker by night, candyman by day. Oncogene, 2008. 27 Suppl 1: p. S53-70.
34. Wang, Y. F., et al., Apoptosis induction in human melanoma cells by inhibition of MEK is caspase-independent and mediated by the Bcl-2 family members PUMA, Bim, and Mcl-1. Clin Cancer Res, 2007. 13(16): p. 4934-42.
35. Jho, E. H., et al., Wnt/β-catenin/Tcf signaling induces the transcription of Axin2, a negative regulator of the signaling pathway. Mol Cell Biol, 2002. 22(4): p. 1172-83.
36. Chia, I. V. and F. Costantini, Mouse axin and axin2/conductin proteins are functionally equivalent in vivo. Mol Cell Biol, 2005. 25(11): p. 4371-6.
37. Cheung, M., et al., Akt3 and mutant V600E B-Raf cooperate to promote early melanoma development. Cancer Res, 2008. 68(9): p. 3429-39.
38. Villanueva, J., et al., Acquired resistance to BRAF inhibitors mediated by a RAF kinase switch in melanoma can be overcome by cotargeting MEK and IGF-1R/PI3K. Cancer Cell, 2010. 18(6): p. 683-95.
39. Tap, W. D., et al., Pharmacodynamic characterization of the efficacy signals due to selective BRAF inhibition with PLX4032 in malignant melanoma. Neoplasia, 2010. 12(8): p. 637-49.
40. Widlund, H. R., et al., β-catenin-induced melanoma growth requires the downstream target Microphthalmia-associated transcription factor. J Cell Biol, 2002. 158(6): p. 1079-87.
41. Biechele, T., et al., Chemical-genetic screen identifies riluzole as an enhancer of Wnt/β-catenin signaling in melanoma. Chemistry and Biology, 2010. 17(11): p. 1177-82.
42. Ellies, D. L., et al., The WNT antagonist cSFRP2 modulates programmed cell death in the developing hindbrain. Development, 2000. 127(24): p. 5285-95.
43. Megason, S. G. and A. P. McMahon, A mitogen gradient of dorsal midline Wnts organizes growth in the CNS. Development, 2002. 129(9): p. 2087-98.
44. Dynek, J. N., et al., Microphthalmia-associated transcription factor is a critical transcriptional regulator of melanoma inhibitor of apoptosis in melanomas. Cancer Res, 2008. 68(9): p. 3124-32.
45. Mikheev, A. M., et al., Dickkopf-1 activates cell death in MDA-MB435 melanoma cells. Biochem Biophys Res Commun, 2007. 352(3): p. 675-80.
46. Sinnberg, T., et al., Suppression of casein kinase 1alpha in melanoma cells induces a switch in β-catenin signaling to promote metastasis. Cancer Res, 2010. 70(17): p. 6999-7009.
47. You, L., et al., An anti-Wnt-2 monoclonal antibody induces apoptosis in malignant melanoma cells and inhibits tumor growth. Cancer Res, 2004. 64(15): p. 5385-9.
48. Kotliarova, S., et al., Glycogen synthase kinase-3 inhibition induces glioma cell death through c-MYC, nuclear factor-kappaB, and glucose regulation. Cancer Res, 2008. 68(16): p. 6643-51.
49. Madhunapantula, S. V. and G. P. Robertson, The PTEN-AKT3 signaling cascade as a therapeutic target in melanoma. Pigment Cell Melanoma Res, 2009. 22(4): p. 400-19.
50. Guger, K. A. and B. M. Gumbiner, A mode of regulation of β-catenin signaling activity in Xenopus embryos independent of its levels. Dev Biol, 2000. 223(2): p. 441-8.
51. Nelson, R. W. and B. M. Gumbiner, A cell-free assay system for β-catenin signaling that recapitulates direct inductive events in the early xenopus laevis embryo. J Cell Biol, 1999. 147(2): p. 367-74.
52. Staal, F. J., et al., Wnt signals are transmitted through N-terminally dephosphorylated beta-catenin. EMBO Rep, 2002. 3(1): p. 63-8.
53. Major, M. B., et al., Wilms tumor suppressor WTX negatively regulates WNT/β-catenin signaling. Science, 2007. 316(5827): p. 1043-6.
54. Smalley, K. S., M. Lioni, and M. Herlyn, Life isn't flat: taking cancer biology to the next dimension. In Vitro Cell Dev Biol Anim, 2006. 42(8-9): p. 242-7.
55. Chou, T. C., Theoretical basis, experimental design, and computerized simulation of synergism and antagonism in drug combination studies. Pharmacol Rev, 2006. 58(3): p. 621-81.

TABLE 1
siRNA sequences used in the described studies

| Target Gene | | Sense Sequence | |
|---|---|---|---|
| Symbol | siRNA ID | 5′ ->3′ | Vendor |
| Control | Control | | Ambion |
| | siRNA #1 | | |
| CTNNB1 | S437 | GGAUGUUCACA | Ambion |
| | | ACCGAAUUtt | |
| AXIN1 | S15814 | GAAAGUACAUU | Ambion |
| | | CUUGAUAAtt | |
| AXIN1 | S15816 | GGAUACCUGCC | Ambion |
| | | GACCUUAAtt | |
| AXIN2 | S15819 | GUAUUACAGCU | Ambion |
| | | ACUCGAAAtt | |
| BRAF | 442 | CCUUUCAGUGCUA | Invitrogen |
| | | CCTTCATCTCTT | |
| BRAF | 1466 | GGAACAGUCUA | Invitrogen |
| | | CAAGGGAAtt | |
| BRAF | 1962 | GCUUUCAGUCA | Invitrogen |
| | | GAUGUAUAtt | |
| BRAF | 2008 | GAUGACUGGACAG | Invitrogen |
| | | UUACCUUAUUCA | |
| BRAF (V600E) | MUT-A | GCTACAGAGAA | Invitrogen |
| | | AUCUCGAUtt | |

TABLE 2
Allelic Status of BRAF^{V600E} in Melanoma Cell Lines

| | | | | SYNERGISTIC |
|---|---|---|---|---|
| | | | SYNERGISTIC | APOPTOSIS |
| | BRAFV600E | | WNT | WITH WNT |
| | ALLELE | REFERENCE | ACTIVATION | AND |
| CELL LINE | STATUS | SOURCE | WITH PLX4720? | PLX4720? |
| A375 | homozygous | Wellcome Trust Sanger | YES | YES |
| | | Institute COSMIC | | |
| | | database* | | |
| COLO829 | heterozygous | Wellcome Trust Sanger | YES | YES |
| | | Institute COSMIC | | |
| | | database* | | |
| MEL624 | heterozygous | Zhao Y. et al., | YES | YES |
| | | Biochemical and | | |
| | | Biophysical Research | | |
| | | Communications | | |
| | | 370(3), 509-513 | | |
| A2058 | heterozygous | Wellcome Trust Sanger | NO | NO |
| | | Institute COSMIC | | |
| | | database* | | |
| SKMEL5 | heterozygous | Wellcome Trust Sanger | NO | NO |
| | | Institute COSMIC | | |
| | | database* | | |
| SKMEL28 | homozygous | Wellcome Trust Sanger | NO | NO |
| | | Institute COSMIC | | |
| | | database* | | |

*Located on the World Wide Web at http://www.sanger.ac.uk/genetics/CGP/CellLines/

TABLE 3
AXIN1/2 siRNAs confer sensitivity to PLX4720-mediated apoptosis in previously unresponsive cell lines.

| | Control siRNA | | | | AXIN1/2 siRNA | | | |
|---|---|---|---|---|---|---|---|---|
| Melanoma | L CM | W3A CM | L CM | W3A CM | L CM | W3A CM | L CM | W3A CM |
| Cell Line | DMSO | DMSO | PLX4720 | PLX4720 | DMSO | DMSO | PLX4720 | PLX4720 |
| A2058 | 2.11 ± 0.28 | 2.00 ± 0.61 | 3.39 ± 0.17 | 4.45 ± 0.41 | 6.4 ± 0.47 | 5.52 ± 1.6 | 12.67 ± 0.89 | 15.07 ± 1.6 |
| SKMEL28 | 2.39 ± 0.63 | 2.02 ± 0.78 | 6.02 ± 0.63 | 8.64 ± 1.9 | 2.57 ± 0.11 | 1.93 ± 0.95 | 31.47 ± 0.13 | 33.93 ± 4.1 |
| SKMEL5 | 2.49 ± 0.92 | 1.81 ± 1.0 | 3.39 ± 0.22 | 4.28 ± 1.2 | 1.97 ± 0.08 | 2.88 ± 0.37 | 11.88 ± 0.18 | 13.13 ± 2.7 |

A2058, SMKEL5, and SKMEL28 cell lines exhibit minimal apoptosis following Wnt/β-catenin activation and targeted BRAF inhibition (FIG. 5B). Cells were transfected with control siRNA or siRNA targeting AXIN1/2. 48 hours following transfection, cells were treated with the indicated conditions for 24 hours and analyzed for apoptosis by flow cytometry for cleaved caspase-3. Apoptosis following 2 uM PLX4720 treatment was significantly enhanced in cells pretreated with siRNAs targeting AXIN1/2. Values represent the average number of cleaved caspase-3-positive cells ± standard deviation in three or more biologic replicates.

What is claimed herein is:

1. A method of treating melanoma in a subject, the method comprising,
administering a therapeutically effective amount of an inhibitor of ERK signaling; and
administering a therapeutically effective amount of an activator of the Wnt/β-catenin signaling pathway.

2. The method of claim 1, further comprising administering to the subject a therapeutically effective amount of a PI3K inhibitor.

3. The method of claim 1, wherein the subject is a human.

4. The method of claim 1 wherein the inhibitor of ERK signaling is selected from the group consisting of inhibitors of ERK1/2, inhibitors of BRAF, inhibitors of a BRAF mutant, inhibitors of BRAF^{V600E} and inhibitors of MEK.

5. The method of claim 1, wherein the inhibitor of a component of ERK signaling is selected from the group consisting of PLX4720, PLX4032 (vemurafenib), AZD6244, GSK2118436 and U0126.

6. The method of claim 1, wherein the activator of the Wnt/β-catenin signaling pathway is a GSK3β inhibitor.

7. The method of claim 6, wherein the GSK3β inhibitor is selected from the group consisting of:
CHIR99021 and CHIR-837.

8. The method of claim 1, wherein the activator of the Wnt/β-catenin signaling pathway is a Wnt ligand.

9. The method of claim 1, wherein the administration of the inhibitor of ERK signaling and the activator of Wnt/β-catenin signaling pathway synergistically increase tumor cell apoptosis.

10. A method of predicting the response of a subject in need of treatment for melanoma to treatment with an inhibitor of ERK signaling and optionally an activator of Wnt/β-catenin comprising:
determining an amount of an AXIN1 protein in a biological sample obtained from the subject; and
comparing the amount to a reference value;
wherein an amount of an AXIN1 protein in the biological sample which is equal to or greater than the reference value indicates that the subject will be less likely to respond to the inhibitor and optionally the activator; and
wherein an amount of an AXIN1 protein in the biological sample which is less than the reference value indicates that the subject will be more likely to respond to the inhibitor and optionally the activator.

11. The method of claim 10, wherein the biological sample is obtained after the subject is administered a dose of an inhibitor of ERK signaling and wherein the reference value is an amount of AXIN1 protein determined in a biological sample obtained from said subject prior to administering said inhibitor of ERK signaling.

12. The method of claim 10, wherein the subject is a human.

13. The method of claim 10, wherein the inhibitor of ERK signaling is selected from the group consisting of inhibitors of ERK1/2, inhibitors of BRAF, inhibitors of a BRAF mutant, inhibitors of BRAF^{V600E} and inhibitors of MEK.

14. The method of claim 10, wherein the inhibitor of ERK signaling is a small molecule inhibitor.

15. The method of claim 10, wherein the inhibitor of ERK signaling is selected from the group consisting of PLX4720, PLX4032 (vemurafenib), AZD6244, GSK2118436 and U0126.

16. The method of claim 10, further comprising administering an inhibitor of ERK signaling and an activator of Wnt/β-catenin signaling to the subject when the level of the AXIN1 gene product is less than the reference value.

17. A method of predicting the response of a subject in need of treatment for melanoma to treatment with an inhibitor of ERK signaling and optionally an activator of Wnt/β-catenin signaling, the method comprising:
determining an amount of a nuclear β-catenin marker in a biological sample obtained from the subject; and
comparing the amount to a reference value;
wherein an amount of a nuclear β-catenin marker in the biological sample which is greater than the reference value indicates that the subject will be more likely to respond to the inhibitor and optionally the activator; and
wherein an amount of a nuclear β-catenin marker in the biological sample which is less than the reference value indicates that the subject will be less likely to respond to the inhibitor and optionally the activator.

18. A method of treating melanoma in a subject, the method comprising:
determining an amount of a nuclear β-catenin marker in a biological sample obtained from the subject; and
comparing the amount to a reference value; and
administering an inhibitor of ERK signaling and optionally an activator of Wnt/β-catenin when the amount of a nuclear β-catenin marker in the biological sample is greater than the reference value, wherein said melanoma is more sensitive to treatment with the inhibitor of ERK signaling than a melanoma with an amount of a marker of nuclear β-catenin that is less than the reference value.

19. A method of treating melanoma in a subject unresponsive to treatment with an inhibitor of ERK signaling and an activator of Wnt/β-catenin signaling, the method comprising,
administering a therapeutically effective amount of an inhibitor of AXIN1;
administering a therapeutically effective amount of an inhibitor of ERK signaling; and
administering a therapeutically effective amount of an activator of the Wnt/β-catenin signaling pathway;
thereby treating melanoma in a subject unresponsive to treatment with an inhibitor of ERK signaling and an activator of Wnt/β-catenin signaling.
